(19) United States
(12) Patent Application Publication
(10) Pub. No.: US 2024/0216336 A1
(43) Pub. Date: Jul. 4, 2024

(54) CLOTRIMAZOLE AS A TREATMENT FOR IMMUNODEFICIENCY DISORDERS

(71) Applicant: Northwestern University, Evanston, IL (US)

(72) Inventors: Hossein Ardehali, Chicago, IL (US); Adam De Jesus, Chicago, IL (US)

(21) Appl. No.: 18/006,688

(22) PCT Filed: Jul. 28, 2021
(86) PCT No.: PCT/US2021/071037

§ 371 Date: Jan. 23, 2024

Related U.S. Application Data
(60) Provisional application No. 63/057,402, filed on Jul. 28, 2020.

(51) Int. Cl. A61K 31/4174; A61K 31/335; A61K 31/365; A61P 37/06

(52) U.S. Cl. CPC A61K 31/4174; A61K 31/335; A61K 31/365; A61P 37/06

(57) ABSTRACT

Disclosed are methods for the treatment of immunodeficiency diseases or disorders in subjects in need thereof by administering to the subject an effective amount of a therapeutic agent that results in dissociation of hexokinase 1 (HK1) from the outer membrane of mitochondria and into the cytosol of macrophage cells in the subject and results in inducing a hyperinflammatory response in the subject.

### CROSS-REFERENCE TO RELATED PATENT APPLICATIONS

The present application represents the national stage entry of PCT/US2021/071037, filed Jul. 28, 2021, which claims the benefit of priority under 35 U.S.C. § 119(e) to U.S. Provisional Application No. 63/057,402, filed on Jul. 28, 2020. The contents of each are hereby incorporated by reference in their entireties.

### STATEMENT REGARDING FEDERALLY SPONSORED RESEARCH OR DEVELOPMENT

This invention was made with government support under HL138982, HL127646, and HL132552 awarded by the National Institutes of Health. The government has certain rights in the invention.

### SEQUENCE LISTING

This application includes a sequence listing in ST.25 format titled “702581.02257_ST.25.txt”, which is 1,899 bytes in size and was created on Jan. 22, 2024. The sequence listing is electronically submitted with this application via Patent Center and is incorporated herein by reference in its entirety.

### BACKGROUND

The field of the invention relates to methods for treating immunodeficiency diseases or disorders in subjects in need thereof. In particular, the field of the invention relates to methods for the treatment of immunodeficiency diseases or disorders in subjects in need thereof by administering to the subject an effective amount of a therapeutic agent that disassociates hexokinase 1 (HK1) from the outer membrane of mitochondria and into the cytosol of macrophage cells in the subject and induces elevated production of inflammatory cytokines including IL-1β, TNFα, and IL-6. The field of the invention also relates to methods for inducing elevated production of inflammatory cytokines including IL-1β, TNFα, and IL-6 in a subject in need thereof.

Immunosuppressive disorders are a major problem in the United States and the number of immunocompromised patients is expected to increase. The spectrum of primary and secondary immunodeficiency disorders is vast and expanding. Medical advances such as organ transplantation and chemotherapy have prolonged the lives of millions, but often at the expense of severe immunodeficiency in the patients. Additionally, primary immunodeficiency disorders encompass a plethora of disease involving the innate and adaptive immune system that often affects children and can lead to premature death in this population. The underlying problem in many of these disorders is an ineffective ability to mount an inflammatory response. This inability to properly active innate and adaptive inflammatory response leaves the patient susceptible to opportunistic infections, which can be fatal. Therefore, it is imperative to find novel therapies to help restore normal inflammatory function in these patients.

In the present application, the inventors describe for the first time that clotrimazole can activate the innate inflammatory response of macrophages in vitro. Clotrimazole was first used as an anti-fungal agent, however, it has been used for a variety of diseases including sickle cell anemia, malaria, Chagas disease, and cancer. Here it is shown that clotrimazole can induce elevated production of inflammatory cytokines including IL-1β, TNFα, and IL-6. These inflammatory cytokines are critical mediators for mounting an immunologic defense against foreign pathogens. Therefore, for the first time clotrimazole is used as a novel therapeutic agent that can be used for boosting the inflammatory response of immunosuppressed patients.

### SUMMARY

Disclosed are methods for treating immunodeficiency diseases or disorders in subjects in need thereof. The disclosed methods of treatment may include methods for treating immunodeficiency diseases or disorders in subjects in need thereof by administering to the subject an effective amount of a therapeutic agent that results in dissociation of hexokinase 1 (HK1) from the outer membrane of mitochondria and into the cytosol of macrophage cells in the subject. In particular, the disclosed methods may include methods for elevating production of inflammatory cytokines in subjects in need thereof by administering to the subject an effective amount of a therapeutic agent that results in dissociation of hexokinase 1 (HK1) from the outer membrane of mitochondria and into the cytosol of macrophage cells in the subject.

Immunodeficiency disease or disorders treated by the disclosed methods may include, but are not limited to, HIV infection, ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglubulinemia, Bruton's disease, congenital agammaglobulinemia, selective deficiency of IgA, or Wiskott-Aldrich syndrome. The immunodeficiency disease or disorder treated by the disclosed methods may also be the result of a family history of primary immunodeficiency, the result of spleen removal, the result of cancer, or the result of liver cirrhosis in the subject in need thereof.

The therapeutic agents that are administered to a subject in the disclosed methods may induce elevated production of IL-1β, TNFα, and IL-6, for example by macrophage cells. The therapeutic agents that are administered to a subject in the disclosed methods may inhibit glyceraldehyde-3-phosphate dehydrogenase (GAPDH) activity, for example in macrophage cells. The therapeutic agents that are administered to a subject in the disclosed methods may increase the activity of the pentose phosphate pathway (PPP), for example in macrophage cells. The therapeutic agents that are administered to a subject in the disclosed methods may induce a hyper-inflammatory response in the subject. Suitable therapeutic agents for use in the disclosed methods that result in the dissociation of HK1 from the outer membrane of mitochondria and into the cytosol may include, but are not limited to, clotrimazole, bifonazole, econazole, ketoconazole, miconazole, and tiocinazole.

The disclosed methods of treatment may include methods for treating immunodeficiency diseases or disorders in subjects in need thereof by administering to the subject an effective amount of a therapeutic agent that the activity of glyceraldehyde-3-phosphate dehydrogenase (GAPDH). The disclosed methods may include methods for elevating production of inflammatory cytokines in subjects in need thereof by administering to the subject an effective amount of a therapeutic agent that inhibits the activity of glyceraldehyde-3-phosphate dehydrogenase (GAPDH). Suitable therapeutic agents that inhibit the activity of GAPDH may include, but are not limited to CGP 3466 maleate (CAS 200189-97-5), heptelidic acid, deprenyl, and dihydromanumycin A.

### BRIEF DESCRIPTION OF THE DRAWINGS

FIG. 1. Generation of HK1 MBD-Deleted Mouse Model. (A) Schematic of HK1 mouse model generation using CRISPR-Cas9. (B) Mouse weights from 3 to 8 weeks (n=5 mice per condition, repeated measures two-way ANOVA, mean±SD). (C-D) Glucose tolerance test (GTT) (B) and insulin tolerance test (ITT) (C) from 10-week-old mice (n=5 mice per condition, repeated measures two-way ANOVA, mean±SD). (E) Mitochondria and cytosolic protein fractionation and western blot of brain, kidney and lung tissue, VDAC1 is a mitochondrial marker and α-tubulin is a cytosolic marker. (F-G) HK activity assay normalized to total protein from brain (F) and lung (G) tissue (n=4 mice per condition, unpaired t-test, mean±SD). (H) Representative immunofluorescence (IF) images probing for HK1 and mitochondria (MT-Red) in isolated BMDMs. (I) Colocalization analysis of IF images (H) using Pearson's correlation coefficient calculated per cell between MT-Red (red) and HK1 (green) image channels (n=10 cells per condition, unpaired t-test, mean±SD, colocalization analysis performed using Coloc-2 macro on ImageJ). (J) 2-NBDG glucose uptake assay normalized to total protein of LPS (200 ng/ml)-activated BMDMs (n=4 mice per condition, unpaired t-test, mean±SD). (K) HK activity assay normalized to total protein of LPS (200 ng/ml)-activated BMDMs (n=5 mice per condition, unpaired t-test, mean±SD).

FIG. 2. Loss of HK1 Mitochondrial Binding Alters Glucose Metabolism and Increases Pentose Phosphate Pathway Intermediates. (A) Extracellular lactate quantification in LPS-activated BMDMs (n=6-9 mice per condition, unpaired t-test, mean±SD). (B) ECAR trace of unstimulated BMDMS±acute LPS (200 ng/ml) stimulation for 2 hrs (n=9 mice per condition, repeated measures two-way ANOVA, mean±SEM). (C) ECAR trace of BMDMS±5 hrs LPS (200 ng/ml) stimulation (n=5 mice per condition, repeated measures two-way ANOVA, mean±SEM). (D) Schematic of ¹³C-glucose carbon labeling through glycolysis (upper and lower glycolysis), PPP, and TCA cycle. Green arrow indicates increased metabolites and red depicts reduced levels of metabolites. (E-F)¹³C-glucose incorporation into upper glycolytic metabolites, G6P (E) and GAP (F), ±4 hrs LPS treatment (n=5 mice per condition, two-way ANOVA, mean±SEM). (G-H)¹³C-glucose incorporation into PPP metabolites, 6-PG (G) and sedoheptulose-7P (H), ±4 hrs LPS treatment (n=5 mice per condition, two-way ANOVA, mean±SEM). (I) NADPH/NADP+ ratio normalized to total protein in isolated BMDMs±4 hr LPS treatment (N=4 mice per condition, one-way ANOVA and Tukey's post-hoc test, mean±SD). (J-L)¹³C-glucose incorporation into lower glycolytic metabolites, 2/3-PG (J), pyruvate (K), and lactate (L), 4 hrs LPS treatment (n=5 mice per condition, two-way ANOVA, mean±SEM). (M-P)¹³C-glucose incorporation into TCA metabolites, citrate (M), αKG (N), succinate (O), and fumarate (P)±4 hrs LPS treatment (n=5 mice per condition, two-way ANOVA, mean SEM).

FIG. 3. Constitutive HK1 Mitochondrial Dissociation Increases Inflammatory Cytokine Production in vitro & in vivo. (A-C) Inflammatory cytokine mRNA expression of IL-1β (A), IL-6 (B), and TNFα (C) from isolated BMDMs±6 hrs LPS (200 ng/ml) (n=4 mice per condition, one-way ANOVA and Tukey's post-hoc test, mean±SD). (D) IL-1β ELISA from BMDM media±6 hrs of LPS (200 ng/ml) followed by 30 min ATP (2.5 mM), normalized to total protein (n=6-7 mice per condition, unpaired t-test, mean±SD). (E-G) mRNA expression of IL-1β (E), IL-6 (F), and TNFα (G) from isolated PMs±6 hrs LPS (300 ng/ml) (n=3 mice per condition, one-way ANOVA and Tukey's post-hoc test, mean±SD). (H) IL-1β ELISA from PM media±6 hrs of LPS (300 ng/ml) followed by 30 min of ATP (2.5 mM), normalized to total protein (n=7-10 mice per condition, unpaired t-test, mean±SD). (I-K) mRNA expression of IL-1β (I), IL-6 (J), and TNFα (K) from splenic tissue after i.p. injection of mice with LPS (15 mg/kg) for 4 hrs. (n=4 mice per condition, one-way ANOVA and Tukey's post-hoc test, mean±SD). (L) Schematic of LPS-induced endotoxemia model. Mice were given i.p. injection of LPS (15 mg/kg) and observed over 72 hrs for survival. (M) Survival curve of mice in LPS-induced endotoxemia model (n=10 mice per condition, survival curve log-rank (Mantel-Cox) test).

FIG. 4. Inhibition of PPP Reverses Hyper-Inflammation Induced by HK1 Mitochondrial Dissociation. (A) Schematic of oxidative and non-oxidative branch of PPP with 6AN blocking the oxidative branch and OT blocking the non-oxidative branch. (B-C) IL-1β ELISA from BMDM media after 4 hrs of LPS (200 ng/ml)±6AN (1 mM) (B) or OT (50 μM) (C) followed by 30 min of ATP (2.5 mM), normalized to total protein (n=3 mice per condition, two-way ANOVA and Tukey's post-hoc test, mean±SD). (D-E) mRNA expression of IL-1β (D) and IL-6 (E)±4 hr 6AN (1 mM) or ±LPS (200 ng/ml) (n=3 mice per condition, two-way ANOVA and Tukey's post-hoc test, mean±SD). (F-G) mRNA expression of IL-1β (F) and IL-6 (G)±4 hr OT (50 μM) or ±LPS (200 ng/ml) (n=3 mice per condition, two-way ANOVA and Tukey's post-hoc test, mean±SD). (H-I) mRNA expression of IL-1β (H) and IL-6 (I) with ±16 hrs IFNγ priming followed by ±4 hrs of LPS (200 ng/ml)±6AN (1 mM) and ±OT (50 μM) (n=4 mice per condition, two-way ANOVA and Tukey's post-hoc test, mean±SD).

FIG. 5. GAPDH Activity is Attenuated in Macrophages with HK1 Mitochondrial Detachment. (A) GAPDH activity normalized to total protein in PMs treated with ±LPS (300 ng/ml) (n=5 mice per condition, two-way ANOVA and Tukey's post-hoc test, mean±SD). (B-C) GAPDH activity normalized to total protein in BMDMs±LPS (200 ng/ml) (B) and ±CGP3466 (GAPDH inhibitor) for 4 hrs (C) (n=8 mice per condition for panel B and n=4 mice per condition for panel C, two-way ANOVA and Tukey's post-hoc test, mean±SD). (D) IL-1β mRNA expression in BMDMs±LPS and ±CGP3466 for 4 hrs (n=3 mice per condition, two-way ANOVA and Tukey's post-hoc test, mean±SD). (E-F) IF of HK1 and mitochondria (MT-Red) imaging in RAW264.7 cells±clotrimazole (CLT) (20 μM) (E) and mitochondrial to HK1 colocalization analysis (F) (n=10 cells per condition, unpaired t-test, mean±SD, colocalization analysis performed using Coloc-2 macro on ImageJ). (G-I) mRNA expression of IL-1β (G), IL-6 (H), and TNFα (I) in RAW264.7 cells±CLT (20 μM) (n=4 replicates per condition, two-way ANOVA and Tukey's post-hoc test, mean±SD). (J) GAPDH activity normalized to total protein in RAW264.7 cells±CLT (20 μM) (n=4 replicates per condition, two-way ANOVA and Tukey's post-hoc test, mean±SD).

FIG. 6. Cytosolic HK1 mediates GAPDH nitrosylation through S100A8/9 binding. (A) Western blot of HK1 co-IP from BMDMs treated with LPS (200 ng/ml) for 3 hrs and probing for S100A8 binding. LE=Low-exposure and HE=high-exposure. (B) HK1 co-IP western blot densitometry analysis of S100A8 divided by total HK1 eluted (n=5 mice per condition, unpaired t-test, mean±SD, densitometry analysis performed using Gel Analyzer on ImageJ). (C-D) Western blot of GAPDH IP and TMT switch nitrosylation assay of LPS (200 ng/ml) treated BMDMs±1400W (10 mg/kg) and Western blot densitometry of anti-TMT normalized to total GAPDH eluted (n=3 mice per condition, one-way ANOVA and Tukey's post-hoc test, mean±SD, densitometry analysis performed using Gel Analyzer on ImageJ). (E) Schematic of in vivo LPS (15 mg/kg)+1400W (10 mg/kg) i.p. injection experiment. (F-H) Spleen tissue mRNA expression of IL-1β (F), IL-6 (G), and TNFα (H) from mice after i.p. injection of LPS±1400W (n=4 mice per condition, one-way ANOVA and Tukey's post-hoc test, mean±SD). (I-K) mRNA expression of IL-1β (I), IL-6 (J), and TNFα (K) from isolated BMDMs±4 hrs LPS (200 ng/ml)+1400W (50 μM) (n=4 mice per condition, one-way ANOVA and Tukey's post-hoc test, mean±SD). (L) GAPDH activity normalized to total protein from BMDMs treated with LPS (200 ng/ml)+1400W (50 μM) (n=4 mice per condition, one-way ANOVA and Tukey's post-hoc test, mean±SD). (M-N) ECAR trace of BMDMs±5 hrs of LPS (200 ng/ml) (M) or +5 hrs LPS±1400W (50 μM) (n=3-4 mice per condition, repeated measures two-way ANOVA and Tukey's post-hoc test, mean±SEM).

FIG. 7. Diabetes and Aging Are Associated with HK1 Mitochondrial Dislocation and Increased Cytokine Production. (A-C) Spleen tissue mRNA expression of IL-1β (A), IL-6 (B), and TNFα (C) from NC and HFD mice (n=6-8 mice per condition, unpaired t-test, mean±SD). (D-E) Western blot analysis of HK1 and HK2 from mitochondrial and cytosolic subcellular fractionation of spleen tissue from NC and HFD mice (D). Cytosolic HK1 to mitochondrial HK1 densitometry quantification (E) (n=5 mice per condition, unpaired t test, mean±SD, densitometry analysis performed using Gel Analyzer on ImageJ). (F) GAPDH activity normalized to total protein in PMs isolated from NC and HFD treated with LPS (300 ng/ml)+1400W (50 μM) for 4 hrs (n=4 mice per condition, unpaired t-test, mean±SD). (G) IL-1P ELISA from media of PMs treated with LPS (300 ng/ml) for 6 hrs and ATP (2.5 mM) for 30 min (n=4 mice per condition, one-way ANOVA and Tukey's post-hoc test, mean±SD). (H) Schematic for mechanism of HK1 mitochondrial dissociation and inflammatory cytokine activation.

FIG. 8. Characteristics of ΔE1HK1 Mice. (A) Schematic of EV, FLHK1, and TrHK1 overexpression lentivirus plasmids. (B-C) Live-cell imaging of GFP-tag fluorescence from EV, FLHK1 and TrHK1 overexpressing HepG2 cells with TMRE (mitochondrial dye) (B) and colocalization analysis of IF images (C) using Pearson's correlation coefficient calculated per cell between GFP-tag (green) and TMRE (red) image channels (n=10 cells per condition, one-way ANOVA and Tukey's post-hoc test, mean±SD, colocalization analysis performed using Coloc-2 macro on Fiji/ImageJ). (D) HK activity assay normalized to total protein from HepG2 cells (n=6 replicates per condition, unpaired t-test, mean±SD). (E) G6P quantification normalized to total protein from HepG2 cells (n=5 replicates per condition, unpaired t-test, mean±SD). (F-G) CBC (F) and WBC differential (G) in WT and ΔE1HK1 mice, as measured using HEMAVET 950 whole blood analyzer (n=3 mice per condition, two-way ANOVA and Tukey's post-hoc test, mean±SD). (H-I) Representative immunofluorescence (IF) images probing for HK1 and mitochondria (ATP5B) in isolated PMs (H). Colocalization analysis of IF images (I) using Pearson's correlation coefficient calculated per cell between ATP5B (red) and HK1 (green) image channels (n=12 cells per condition, unpaired t-test, mean±SD, colocalization analysis performed using Coloc-2 macro on Fiji/ImageJ). (J) mRNA expression of all known HK isozymes (HK1, HK2, HK3, GCK, HKDC1) in WT BMDMs (n=5 mice per condition, repeated measures two-way ANOVA and Tukey's post-hoc test, mean±SD). (K-L) mRNA expression of inflammatory (IL-1β and MCP1) (K) and anti-inflammatory markers (ARG2 and PPARγ) (L) in WT BMDMs (n=5 mice per condition, repeated measures two-way ANOVA and Tukey's post-hoc test, mean±SD).

FIG. 9. Overexpression of TrHK1 in HepG2 Cells Shifts Glucose to PPP. (A) Extracellular lactate quantification in HepG2 cells with overexpression of FLHK1 or TrHK1 (n=6 replicates per condition, unpaired t-test, mean±SD). (B) ECAR trace of HepG2 cells with overexpression of FLHK1 or TrHK1 (n=5-8 replicates per condition, repeated measures two-way ANOVA, mean±SD). (C) Schematic of ¹³C-glucose carbon labeling through glycolysis (upper and lower glycolysis), PPP, de novo nucleotide synthesis and TCA cycle. (D)¹³C-glucose incorporation into upper glycolytic metabolites, GAP/DHAP and glycerate in HepG2 cells with overexpression of EV, FLHK1 or TrHK1 (n=3 replicates per condition, two-way ANOVA and Tukey's post-hoc test, mean±SEM). (E)¹³C-glucose incorporation into PPP metabolite sedoheptulose-7P and de novo nucleotides; ADP, AMP, ATP, dGDP, and GTP in HepG2 cells with overexpression of EV, FLHK1 or TrHK1 (n=3 replicates per condition, two-way ANOVA and Tukey's post-hoc test, mean±SEM). (F) NADPH/NADP+ ratio normalized to total protein in HepG2 cells with overexpression of FLHK1 or TrHK1 (N=5 replicates per condition, unpaired t-test, mean±SD). (G)¹³C-glucose incorporation into lower glycolytic metabolites, 2/3-PG, pyruvate, and lactate, ±4 hrs LPS treatment in HepG2 cells with overexpression of EV, FLHK1 or TrHK1 (n=3 replicates per condition, two-way ANOVA and Tukey's post-hoc test, mean±SD). (H)¹³C-glucose incorporation into TCA metabolites, citrate, succinate, and fumarate in HepG2 cells with overexpression of EV, FLHK1 or TrHK1 (n=3 replicates per condition, two-way ANOVA and Tukey's post-hoc test, mean±SEM).

FIG. 10. ΔEH1K1 Mice Display Higher Inflammatory Cytokine Production than WT Mice. (A-B) HK1 (A) and HK2 (B) mRNA expression in isolated BMDMs±6 hrs LPS (200 ng/ml) (n=3-4 mice per condition, one-way ANOVA and Tukey's post-hoc test, mean±SD). (C-D) Anti-inflammatory cytokine mRNA expression of PPARγ (C) and CARKL (D) from isolated BMDMs±6 hrs LPS (200 ng/ml) (n=3-4 mice per condition, one-way ANOVA and Tukey's post-hoc test, mean±SD). (E) Western blot analysis of BMDMs treated with LPS or LPS+ATP probing for Flag-tag, HK1 and pro-IL-1β. (F) IL-1β ELISA from BMDM media±6 hrs of LPS (200 ng/ml) followed by 30 min ATP (2.5 mM), normalized to total protein (n=2-4 mice per condition, one-way ANOVA and Tukey's post-hoc test, mean±SD). (G) Western blot of cleaved-IL-1β from media of LPS or LPS+ATP treated cells.

FIG. 11. Constitutive HK1 Mitochondrial Dislocation Does Not Alter mRNA Expression of Metabolic Genes Downstream of GAPDH (A) Schematic of upper and lower glycolysis, PPP, TCA cycle, and pyruvate metabolic pathways. (B) mRNA expression of upper glycolytic genes HK1, glucose-6-phosphate isomerase (GPI), phosphofructokinase (PFK), and aldolase (ALDO) from isolated BMDMs±6 hrs LPS (200 ng/ml) (n=mice per condition, one-way ANOVA and Tukey's post-hoc test, mean±SD). (C) mRNA expression of lower glycolytic genes GAPDH, triosephosphate isomerase (TPI), Phosphoglucomutase (PGM), phosphoglycerate kinase (PGK), enolase (ENO1), pyruvate kinase (PK) and lactate dehydrogenase A (LDHA) from isolated BMDMs±6 hrs LPS (200 ng/ml) (n=mice per condition, one-way ANOVA and Tukey's post-hoc test, mean±SD). (D) mRNA expression of PPP genes glucose-6-phosphate dehydrogenase (G6PD), phosphogluconate dehydrogenase (PGD), ribulose-5-phosphate-3-epimerase (RPE), ribose-5-phosphate isomerase A (RPIA), transketolase (TKT), and transaldolase (TALDO) from isolated BMDMs±6 hrs LPS (200 ng/ml) (n=mice per condition, one-way ANOVA and Tukey's post-hoc test, mean±SD). (E) mRNA expression of pyruvate metabolism genes pyruvate dehydrogenase E1 alpha 1 subunit (PDHA1), pyruvate dehydrogenase E1 beta subunit (PDHB), dihydrolipoamide dehydrogenase (DLD), (PC), mitochondrial pyruvate carrier 1 (MPC1), and mitochondrial pyruvate carrier 2 (MPC2) from isolated BMDMs±6 hrs LPS (200 ng/ml) (n=mice per condition, one-way ANOVA and Tukey's post-hoc test, mean±SD). (F) mRNA expression of TCA cycle genes isocitrate dehydrogenase (IDH), oxoglutarate dehydrogenase (OGDH/αKGD), citrate synthase (CS), fumarate hydratase (FH), succinate dehydrogenase complex subunit C (SDHC), and malate dehydrogenase (MDH) from isolated BMDMs±6 hrs LPS (200 ng/ml) (n=mice per condition, one-way ANOVA and Tukey's post-hoc test, mean±SD).

FIG. 12. HK1 Dislocation from Mitochondria Leads to Decreased GAPDH Activity. (A) GAPDH activity normalized to total protein in RAW264.7 cells treated with LPS (200 ng/ml) and ±CGP3466 (n=3 replicates per condition, unpaired t-test, mean±SD). (B) IL-1β mRNA expression in RAW264.7 cells±LPS (200 ng/ml) and ±CGP3466 for 4 hrs (n=4 replicates per condition, one-way ANOVA and Tukey's post-hoc test, mean±SD). (C-E) Expression of IL-1β (C), IL-6 (D), and TNFα (E) from isolated BMDMs treated with 6 hrs LPS (200 ng/ml) (n=4 replicates per condition, one-way ANOVA and Tukey's post-hoc test, mean±SD). (F) Schematic of branch points of G6P and glycolytic pathway above and below the level of GAPDH. Green arrow indicates increased metabolites and red depicts reduced levels of metabolites. (G) Heatmap of steady-state metabolomics performed in HepG2 cells with FLHK1 and TrHK1 overexpression all normalized to EV metabolite levels and total-iron-content in the cells (heatmap generated with MetaboAnalyst). (H) NADH/NAD+ ratio measurement derived from steady-state metabolomics peak-mass-spectrometry (MS) values for NADH and NAD+ (n=4 mice per condition, unpaired t-test, mean±SD). (I) Energy charge of the cell calculated based on steady-state metabolomics data using ATP, AMP, and ADP metabolite peak-MS values. Formula for energy charge

$$\frac{[ ATP ] + 0.5 [ ADP ]}{[ ATP ] + [ ADP ] + [ AMP ]}$$

$$( n = 4 mice per condition , unpaired t - test , mean \pm S D ) .$$

(J) GAPDH activity normalized to total protein in HepG2 cells between FLHK1 and TrHK1 normalized to EV (n=6 replicates per condition for, unpaired t-test, mean±SD). (K) Heatmap of steady-state metabolomics performed on whole mouse brain normalized to total-iron-content in the cells (heatmap generated with MetaboAnalyst).

FIG. 13. Cytosolic HK1 Binds to S100A8/9. (A) Venn diagram of total unique spectra identified in co-IP and MS proteomics of HepG2 cell with overexpression of EV, FLHK1, and TrHK1. (B) PANTHER Biological GO-enrichment analysis of proteins identified by proteomics. (C) Table of selected proteins identified by MS with HK1, VDAC and S100 family proteins highlighted with red borders. (D) Peptide spectra of S100A8 and S100A9 identified by MS. (E-F) Co-IP of GFP-tagged EV, FLHK1, and TrHK1 proteins in HepG2 cells and western blot analysis probing for S100A8. (G) Western blot of GAPDH IP and TMT switch nitrosylation assay in HepG2 cells (n=2 replicates per condition). (H) Mouse serum cytokine analysis using ELISA for IL-1β, IL-6, and TNFα from in vivo LPS treated mice±1400W (10 mg/kg). All HepG2 co-IP and MS data was analyzed using Scaffold Proteome Software (A), (C), and (D).

FIG. 14. Diabetes and Aging Lead to Increased Cytosolic HK1 and Inflammatory Cytokine Production. (A) Mouse weights from 3 to 8 weeks after NC and HFD (n=4-7 mice per condition, repeated measures two-way ANOVA, mean±SD). (B) Glucose tolerance test (GTT) from NC and HFD mice (n=5 mice per condition, repeated measures two-way ANOVA, mean±SD). (C-E) mRNA analysis of MCP1 (C), IL-6 (D), and TNFα (E) in adipose tissue (n=3-4 mice per condition, unpaired t-test, mean±SD). (F-H) mRNA analysis of IL-1β (F), IL-6 (G), and TNFα (H) in liver tissue (n=3-4 mice per condition, unpaired t-test, mean±SD). (I-J) Western blot analysis of HK1 from mitochondrial and cytosolic subcellular fractionation of adipose (I) and liver (J) tissue from NC and HFD mice (n=3 mice per condition). (K-L) mRNA expression of IL-1β (K) and IL-6 (L) in spleen tissue from young and aged mice (85-weeks-old) (n=5 mice per condition, unpaired t-test, mean±SD). (M-N) Representative western blot of mitochondrial and cytosolic subcellular fractionation of spleen tissue from young and aged mice (M) and cytosolic HK1 to mitochondrial HK1 densitometry quantification (N) (n=5 mice per condition, unpaired t-test, mean±SD, densitometry analysis performed using Gel Analyzer on Fiji/ImageJ).

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject” may be interchangeable with “patient” or “individual” and means an animal, which may be a human or non-human animal, in need of treatment, for example, treatment by include administering an effective amount of one or more therapeutic agents that results in dissociation of hexokinase 1 (HK1) from the outer membrane of the mitochondria and into the cytosol of macrophage cells.

A “subject in need of treatment” or a “subject in need” may include a subject having or at risk for developing an immunodeficiency disease or disorder. In particular, a subject in need of treatment may include a subject having or at risk for developing an immunodeficiency disease or disorder selected from HIV infection, ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglubulinemia, Bruton's disease, congenital agammaglobulinemia, selective deficiency of IgA, or Wiskott-Aldrich syndrome. A subject in need of treatment may also include a subject having or at risk for developing an immunodeficiency disease or disorder which is the result of a family history of primary immunodeficiency. A subject in need of treatment may also include a subject having or at risk for developing an immunodeficiency disease or disorder which is the result of spleen removal in a subject. A subject in need of treatment may also include a subject having or at risk for developing an immunodeficiency disease or disorder which is the result of cancer or liver cirrhosis in a subject. A subject in need a treatment may include a subject in need of elevated production of inflammatory cytokines including IL-1β, TNFα, and IL-6, and/or a subject that has a disease or disorder that is treated by elevated production of inflammatory cytokines including IL-1β, TNFα, and IL-6.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

In the disclosed methods, a subject in need thereof may be administered an effective amount of a therapeutic agent that results in results in dissociation of hexokinase 1 (HK1) from the outer membrane of mitochondria and into the cytosol of macrophage cells in a subject. Suitable therapeutic agents that may be effective for dissociating hexokinase 1 (HK1) from the outer membrane of the mitochondria into the cytosol of macrophage cells in a subject include clotrimazole, bifonazole, econazole, ketoconazole, miconazole, and tioconazole.

In the disclosed methods, a subject in need thereof may be administered an effective amount of a therapeutic agent that inhibits the activity of glyceraldehyde-3-phosphate dehydrogenase (GADPH). As disclosed herein, the term “inhibiting” may include blocking enzyme activity and/or reducing enzyme activity. Suitable therapeutic agents that may inhibit the activity of glyceraldehyde-3-phosphate dehydrogenase (GADPH) in a subject include CGP 3466 maleate (CAS 200189-97-5), heptelidic acid, deprenyl, and dihydromanumycin A.

The disclosed therapeutic methods may be effective for elevating production of inflammatory cytokines in a subject. As disclosed herein, the term “elevating” may include increasing production and/or concentration of inflammatory cytokines in macrophage cells. Inflammatory cytokines include IL-1β, TNFα, and IL-6.

In the disclosed methods, a subject in need thereof may be administered an effective amount of a therapeutic agent that results in results in elevating production of inflammatory cytokines in subjects in need thereof, such as IL-1β, TNFα, and IL-6. Suitable therapeutic agents that may be effective for elevating production of inflammatory cytokines in subjects in need thereof include clotrimazole, bifonazole, econazole, ketoconazole, miconazole, and tioconazole. Suitable therapeutic agents that may be effective for elevating production of inflammatory cytokines in subjects in need thereof also may include CGP 3466 maleate (CAS 200189-97-5), heptelidic acid, deprenyl, and dihydromanumycin A.

### Clotrimazole as a Treatment for Immunodeficiency Disorders

The disclosed subject matter relates to methods for treating immunodeficiency diseases or disorders in subjects in need thereof. Suitable subjects for the disclosed methods may include subjects having an immunodeficiency disease or disorder. Immunodeficiency diseases or disorders may include, but are not limited to, an HIV infection, ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglubulinemia, Bruton's disease, congenital agammaglobulinemia, selective deficiency of IgA, or Wiskott-Aldrich syndrome. Suitable subjects for the disclosed methods also may include subjects having an immunodeficiency disease or disorder which is the result of a family history of primary immunodeficiency. Suitable subjects for the disclosed methods may also include subjects having an immunodeficiency disease or disorder which is the result of spleen removal in a subject. Suitable subjects for the disclosed methods may also include subjects having an immunodeficiency disease or disorder which is the result of cancer or liver cirrhosis in a subject.

In the disclosed methods, a subject in need thereof may be administered an effective amount of a therapeutic agent that results in dissociation of hexokinase 1 (HK1) from the outer membrane of mitochondria and into the cytosol of macrophage cells in the subject. Suitable therapeutic agents may include, but are not limited to, clotrimazole, bifonazole, econazole, ketoconazole, miconazole, and ticonazole. In particular embodiments, the therapeutic agent is clotrimazole.

In some embodiments of the disclosed methods, the effective amount of the therapeutic agent (e.g., clotrimazole) may induce elevated production of inflammatory cytokines by the macrophage cells. In particular embodiments, the inflammatory cytokines are of IL-1β, TNFα, and IL-6.

In some embodiments, the effective amount of the therapeutic agent (e.g., clotrimazole) may inhibit GAPDH activity in macrophage cells. In some embodiments, the effective amount of the therapeutic agent (e.g., clotrimazole) results in an increase of activity of the pentose phosphate pathway (PPP) in macrophage cells. In some embodiments, the effective amount of the therapeutic agent clotrimazole, induces a hyper-inflammatory response in a subject, which may include but is not limited to an increase in production of inflammatory cytokines such as IL-1β, TNFα, and IL-6.

In the disclosed methods, a subject in need thereof may be administered an effective amount of a therapeutic agent that inhibits the activity of glyceraldehyde-3-phosphate dehydrogenase (GAPDH). Suitable therapeutic agents that inhibit GAPDH activity may include, but are not limited to, CGP 3466 maleate (CAS 200189-97-5), heptelidic acid, deprenyl, and dihydromanumycin A.

The disclosed subject matter also includes methods for elevating production of inflammatory cytokines in a subject in need thereof are disclosed herein. In the disclosed methods, a subject in need thereof for elevating production of inflammatory cytokines may be administered an effective amount of a therapeutic agent that results in dissociation of hexokinase 1 (HK1) from the outer membrane of mitochondria and into the cytosol of macrophage cells in the subject. In some embodiments, the inflammatory cytokines are IL-1β, TNFα, and IL-6. Suitable therapeutic agents may include but are not limited to clotrimazole, bifonazole, econazole, ketoconazole, miconazole, and ticonazole. In particular embodiments, the therapeutic agent is clotrimazole.

In the disclosed methods, a subject in need thereof for elevating production of inflammatory cytokines may be administered an effective amount of a therapeutic agent that inhibits the activity of glyceraldehyde-3-phosphate dehydrogenase (GAPDH). Suitable therapeutic agents may include but are not limited to CGP 3466 maleate (CAS 200189-97-5), heptelidic acid, deprenyl, and dihydromanumycin A.

### EXAMPLES

The following Examples are illustrative and should not be interpreted to limit the scope of the claimed subject matter.

### Example 1—Hexokinase 1 Subcellular Localization Regulates the Metabolic Fate of Glucose

### Introduction

Hexokinases (HKs) represent the first committed step in glucose utilization by catalyzing the phosphorylation of glucose to glucose-6-phosphate (G6P) which sequesters glucose in the cell and commits it to various downstream metabolic pathways (Gould and Holman, 1993; Bell et al., 1993; Mueckler, 1994; Printz et al., 1997). G6P can enter glycolysis for energy production, the pentose phosphate pathway (PPP) for anabolic intermediates, the hexosamine biosynthesis pathway (HBP) for protein glycosylation, or be converted to glucose-1-phosphate for glycogen synthesis (Adeva-Andany et al., 2016; Puleston et al., 2017). In mammals, five HK isozymes (HK1, HK2, HK3, glucokinase (GCK), and hexokinase domain-containing 1 (HKDC1)) have been identified, each with distinct patterns of tissue expression, subcellular localization, enzyme kinetics, and substrate specificities (Middleton, 1990; Ureta, 1982; Wilson, 2003). The molecular weights of HK1, 2, 3, and HKDC1 are ˜100 kDa, whereas GCK and yeast HKs have a molecular weight of 50 kDa. The protein and gene structure of the 100 kDa enzymes suggest that they evolved from gene duplication and fusion of an ancestral yeast-like 50 kDa enzyme (Cirdenas et al., 1998; Printz et al., 1997). Upon the duplication process, the N-terminal half of HK1 and HK3 became enzymatically inactive, whereas HK2 maintained activity in both of its catalytic domains (Ardehali et al., 1996).

HK1 and HK2 also contain an N-terminal, 21-amino-acid hydrophobic sequence (Rose and Warms, 1967; Sui and Wilson, 1997) that enables outer mitochondrial membrane (OMM) binding, possibly through its interaction with the family of mitochondrial bound voltage dependent anion channel (VDAC) proteins (Fiek et al., 1982; Linden et al., 1982; Aflalo and Azoulay, 1998; Azoulay-Zohar et al., 2004). This sequence, termed the mitochondrial binding domain (MBD), is encoded in exon 1, while exons 2 through half of exon 10 encode for the N-terminal domain and the remaining part of exon 10 through exon 18 encode for the C-terminal domain (Printz et al., 1995, 1993). Notably, HK1 and HK2 differ in their affinity for mitochondrial localization (Calmettes et al., 2013; John et al., 2011), with HK1 predominantly bound to the OMM, while HK2 is in a dynamic balance between the mitochondria and cytosol that is regulated, in part, by insulin signaling (Pastorino et al., 2005; Roberts et al., 2013). HK3 and GCK lack a corresponding N-terminal sequence and as a consequence are predominantly cytoplasmic (Wilson, 1995; Cirdenas et al., 1998), although some reports suggest that HK3 may bind to the nuclear envelope (Preller and Wilson, 1992).

Metabolic adaptations are inextricably linked to the immediate host defense against foreign pathogens (Pearce and Pearce, 2013; Sancho et al., 2017; Stienstra et al., 2017). In the setting of innate inflammatory activation, there is a robust increase in glycolysis even in the setting of abundant oxygen, a metabolic program shared by rapidly proliferating cells called aerobic glycolysis (Cheng et al., 2014; Everts et al., 2014; Garaude et al., 2016; O'Neill and Pearce, 2016; Pavlova and Thompson, 2016; Andrejeva and Rathmell, 2017). In addition to elevated glycolytic rate, there is increased glucose flux into the PPP and other ancillary metabolic pathways in immune cells following toll-like receptor (TLR) stimulation (Puleston et al., 2017; Hughes and O'Neill, 2018). In macrophages, HK1 mRNA is highly expressed after pro-inflammatory stimulation (Nishizawa et al., 2014), and HK1-dependent glycolysis is important for proper inflammasome activation in M1 macrophages (Moon et al., 2015). Moreover, HK1 has also been shown to facilitate metabolite flux into the mitochondria in CD8+ cells (Bantug et al., 2018). HK2 is also shown to play an important role in inflammasome activation (Wolf et al., 2016), and in viral-mediated inflammation (Zhang et al., 2019). These results indicate a major role for glucose metabolism through HK1 in the activation of inflammatory cells.

Despite extensive research on glucose metabolism, it remains unclear what factor(s) decide the fate of glucose and regulate the entrance of G6P into a specific pathway. Elucidating this regulation will be important for developing rational therapeutic approaches to inflammatory diseases, cancer, and a host of other immune system related pathologies. Here, we demonstrate that the binding of HK1 to the mitochondria determines whether the product of the enzyme (G6P) is catabolized through glycolysis or shunted through PPP. We demonstrate that constitutive dislocation of HK1 to the cytoplasm shifts G6P entry into the PPP, resulting in higher cytokine production and exaggerated inflammatory response to endotoxemia. The mechanism for the altered G6P metabolism by HK1 cellular distribution is through cytosolic HK1 interaction with S100A8/9, which induces S-nitrosylation of GAPDH and its subsequent inactivation. Therefore, HK1 functions as an important metabolic switch between catabolic and anabolic metabolism through its subcellular localization.

### Results

### Generation of HK1 MBD-Deleted Mouse Model

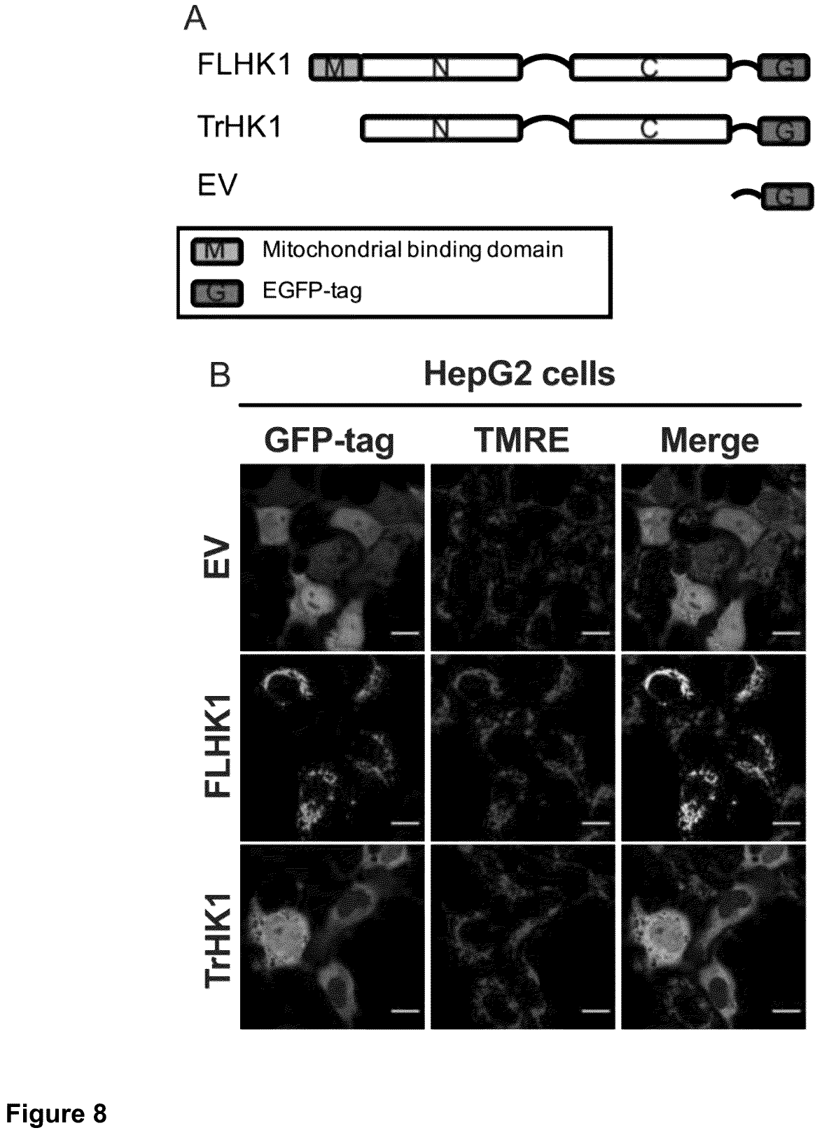

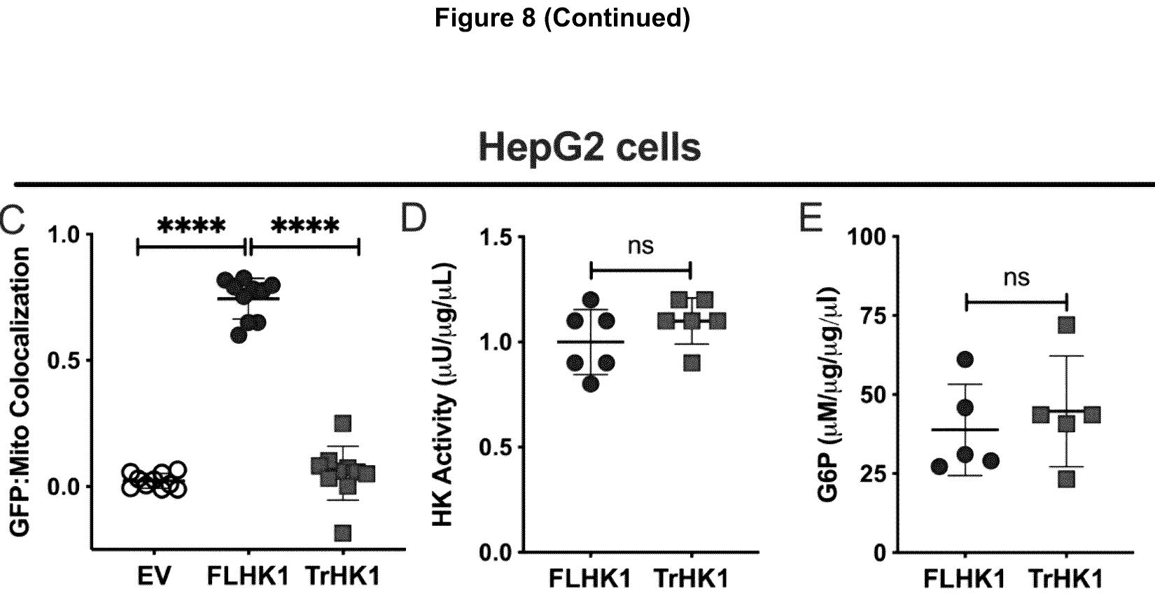

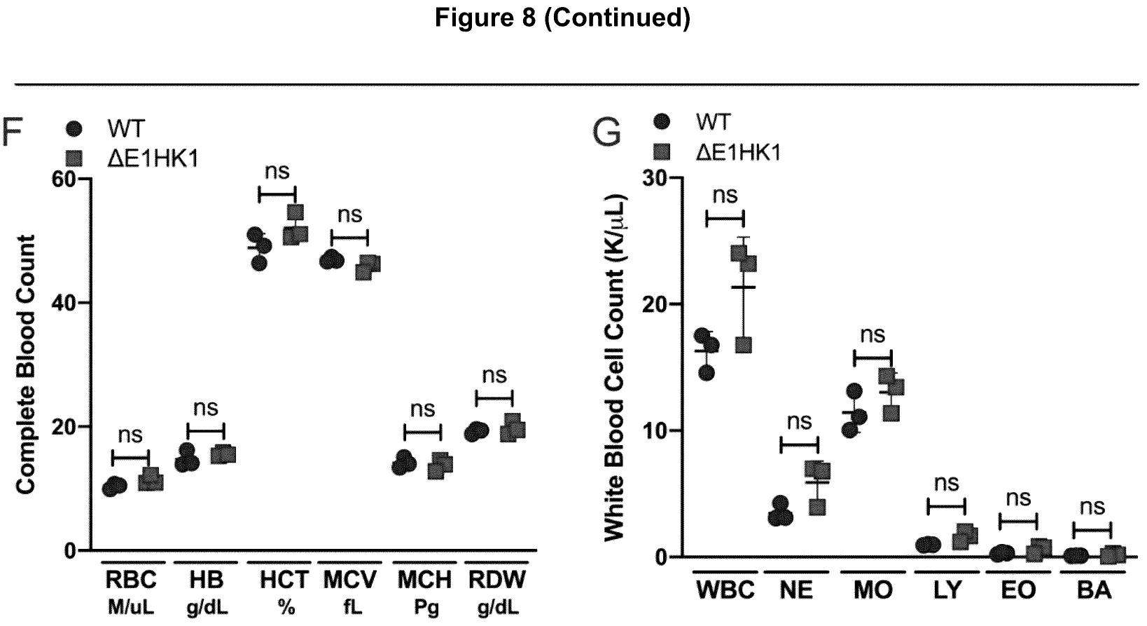

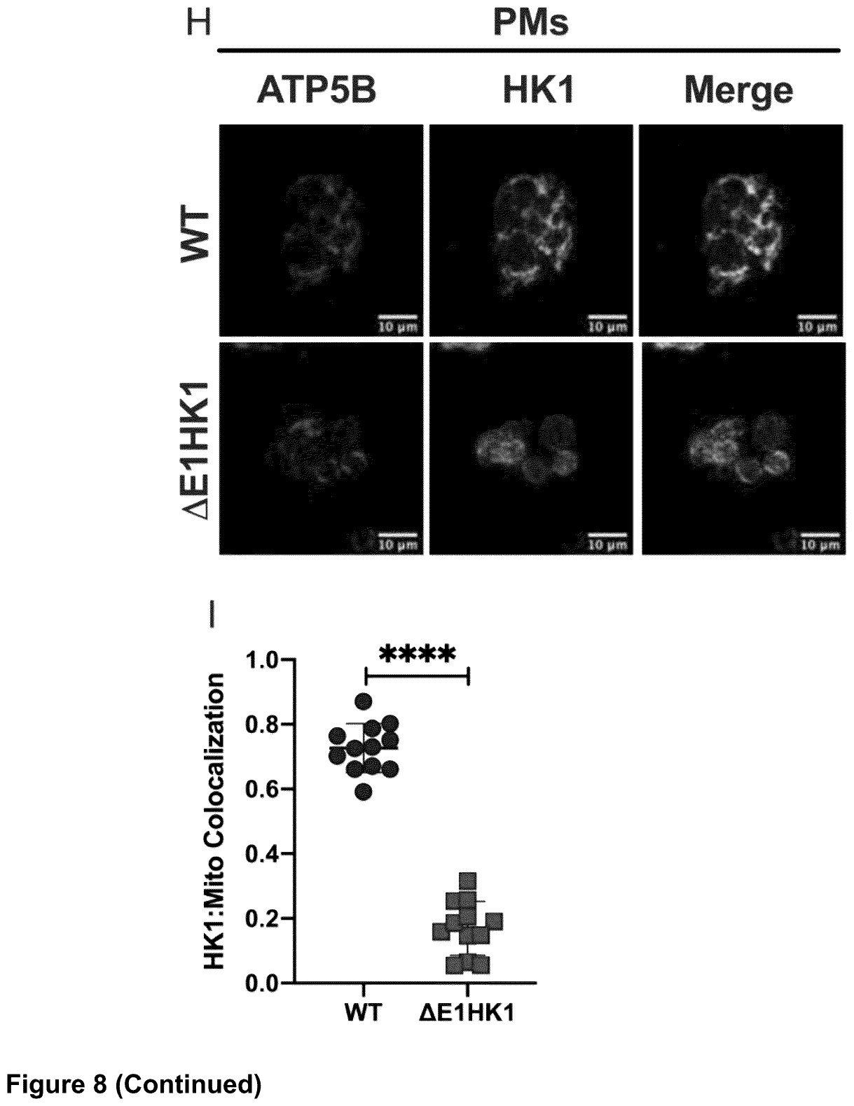

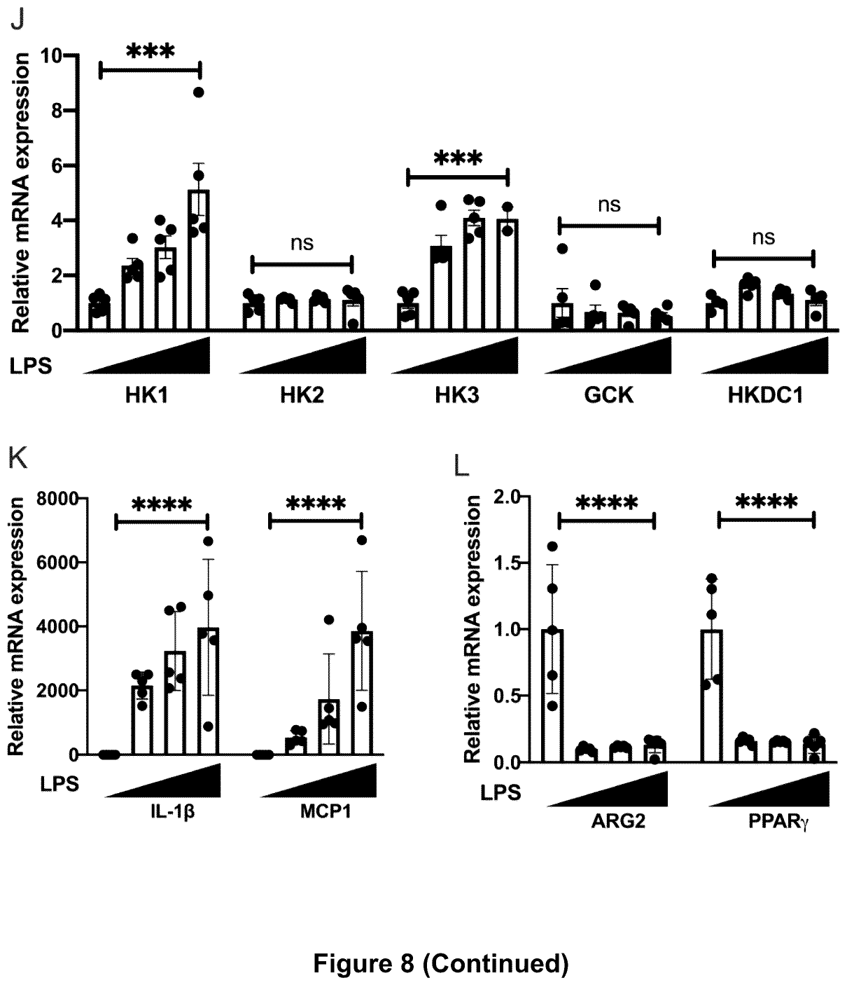

HK1 contains a 21-amino acid hydrophobic N-terminal domain that confers outer-mitochondrial membrane (OMM) binding. To determine the cellular consequences of HK1 mitochondrial binding, we generated GFP-tagged constructs of full-length HK1 (FLHK1) and truncated HK1 (TrHK1) that lack the MBD, along with an empty vector (EV) control that lacks an insert (FIG. 8A). These constructs were transfected into HepG2 cells, and their cellular distribution was assessed by GFP fluorescence. As expected, FLHK1 localized to the mitochondria, while TrHK1 displayed diffuse cellular distribution (FIGS. 8B and 8C). TrHK1 displayed similar HK activity as FLHK1 (FIGS. 8D and 8E), confirming that deletion of the MBD does not affect the ability of HK1 to phosphorylate glucose. These results indicate that altering the subcellular localization of HK1 by removing the MBD does not impair its enzyme activity.

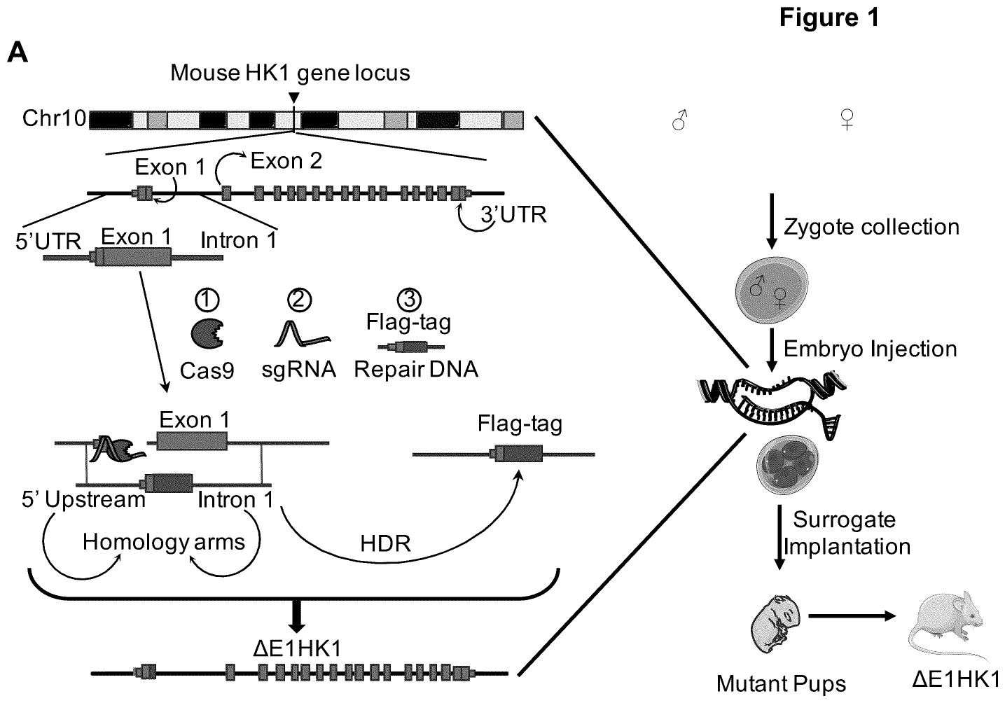

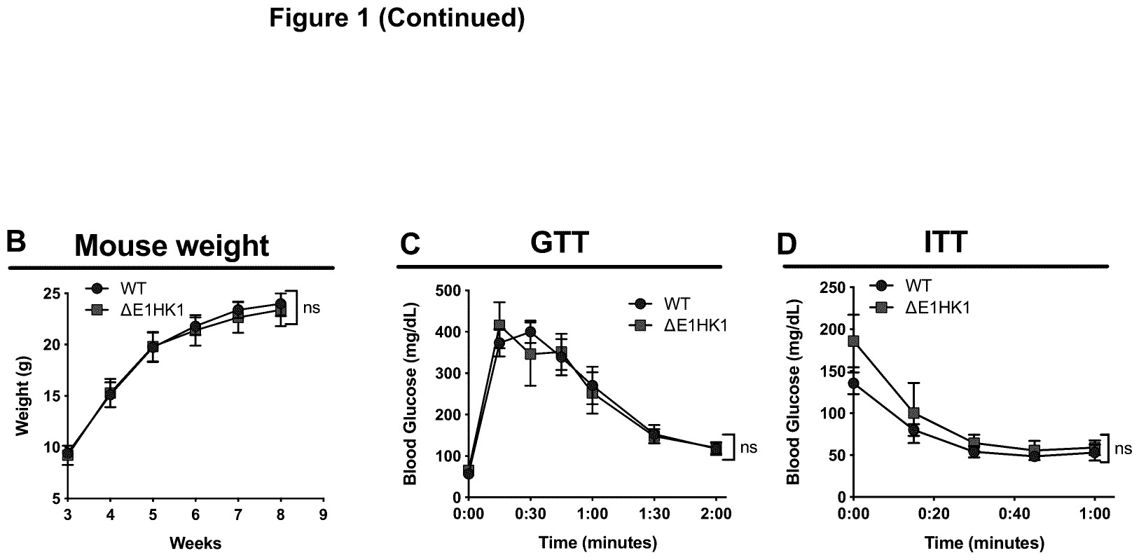

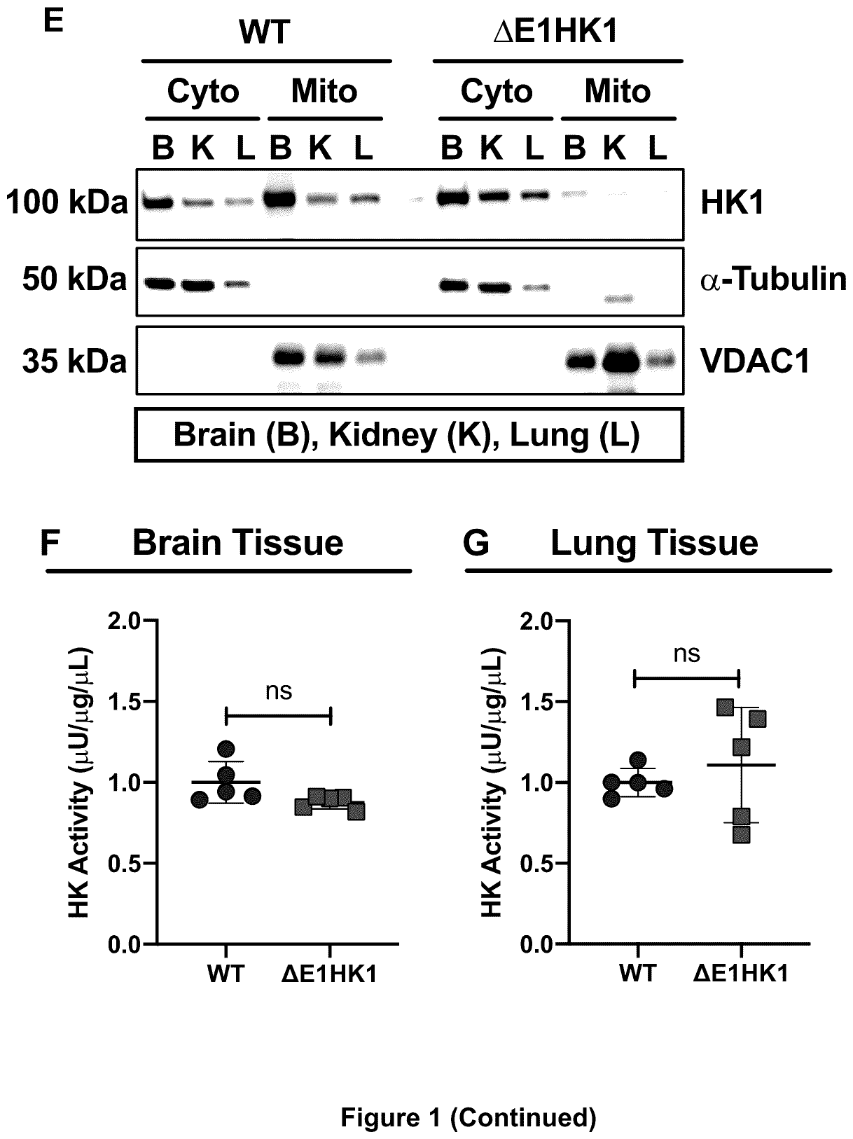

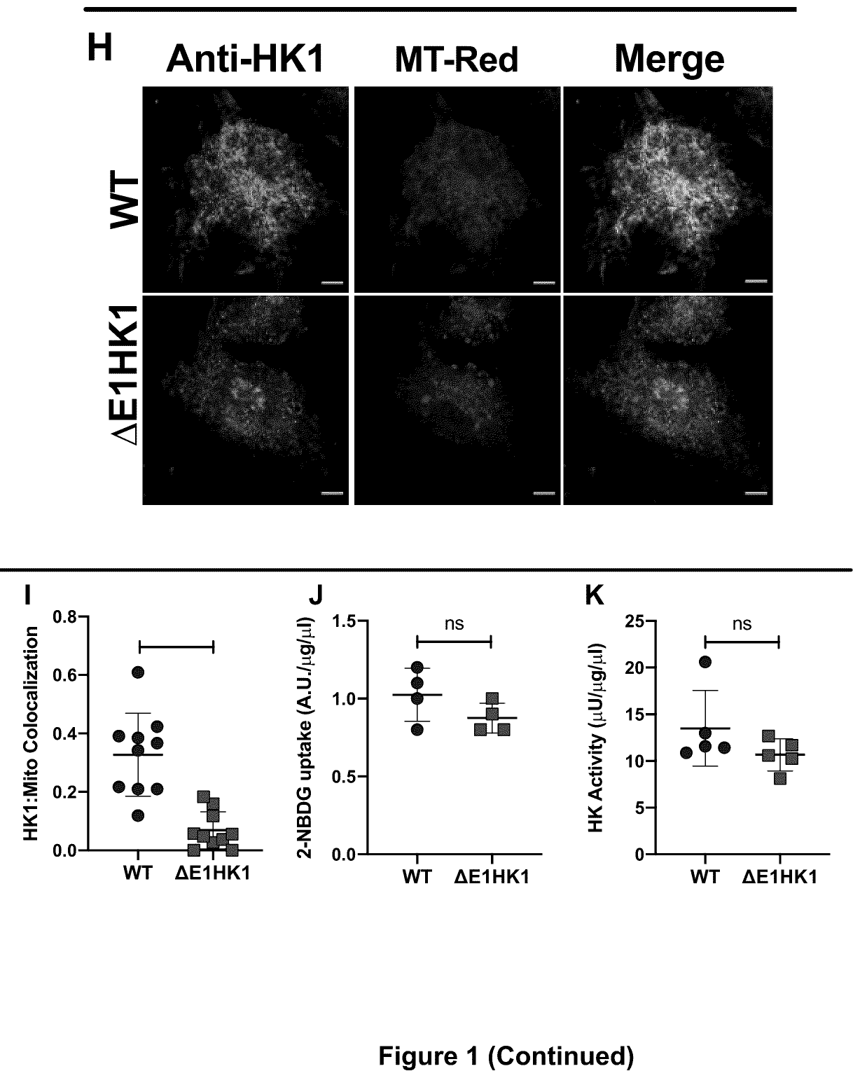

To determine the physiologic function of the HK1 MBD, we generated a mouse model lacking the MBD of the endogenous HK1. The approach to remove the MBD is depicted in FIG. 1A. Guide RNAs (gRNAs) were designed to induce a double strand break (DSB) near the 5′ ATG start codon of HK1 in exon 1, and a DNA template containing an M-DYKDDDDK (SEQ ID NO: 1) (Methionine-FLAG-tag) sequence with flanking homology arms to the HK1 gene near exon 1 was introduced into fertilized zygotes and implanted into surrogate mice. Homology-directed repair (HDR) of the DSB resulted in replacement of the coding region of the HK1 MBD with the Methionine-FLAG-tag sequence (FIG. 1A). Homozygote mice with this mutation, designated as ΔE1HK1 herein, were born in normal Mendelian ratios and displayed no overt changes in weight or difference in whole body glucose homeostasis as assessed by glucose tolerance test (GTT) and insulin tolerance test (ITT) compared to wild type (WT) control mice (FIG. 1B-D). Moreover, hematologic analysis of whole blood from these mice revealed no difference in complete blood cell count (CBC) or white blood cell (WBC) differential (FIGS. 8F and 8G). To assess whether removal of the endogenous HK1 MBD alters cellular distribution of the protein, we isolated mitochondrial and cytosolic fractions from solid organs with high HK1 expression (brain, kidney and lungs), and showed that HK1 was predominantly present in the cytosolic fraction in organs isolated from ΔE1HK1 mice (FIG. 1E). We also performed HK activity assay on whole brain and lung tissue lysates and found no difference in activity between the ΔE1HK1 and WT mice (FIGS. 1F and 1G).

Immunofluorescence (IF) studies in bone marrow-derived macrophages (BMDMs) and peritoneal macrophages (PMs) isolated from WT and ΔE1HK1 mice also confirmed lack of HK1 localization to the mitochondria (FIGS. 1H-1I and 8H-8I). Among the five different HK isoforms, only HK1 and HK3 displayed a dose-dependent increase in their mRNA levels in response to lipopolysaccharide (LPS) in WT BMDMs (FIG. 8J-8L). Additionally, BMDMs isolated from WT and ΔE1HK1 mice displayed similar glucose uptake and phosphorylating activity, consistent with our findings in solid organs from ΔE1HK1 mice and HepG2 overexpression cells (FIGS. 1J and 1K). These results suggest an important role for HK1 in LPS-activated macrophages and provides an ideal system to study the MBD of HK1 in the absence of confounding changes to other mitochondrial bound HKs.

### Loss of HK1 Mitochondrial Binding Alters Glucose Metabolism and Increases Pentose Phosphate Pathway Intermediates

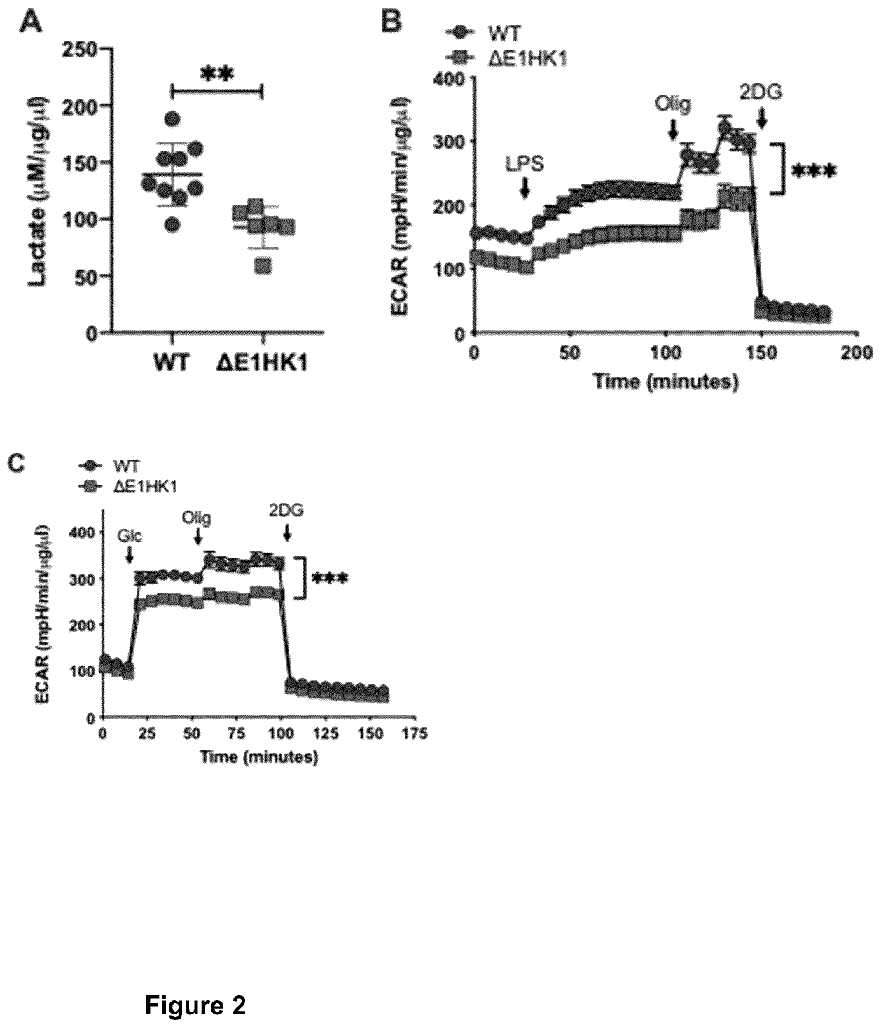

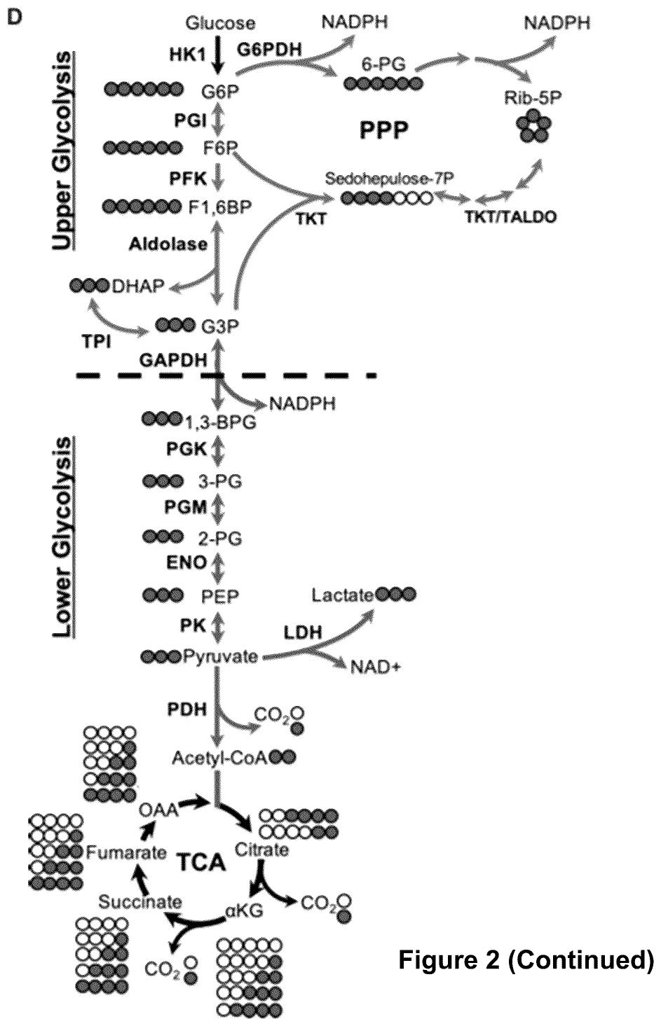

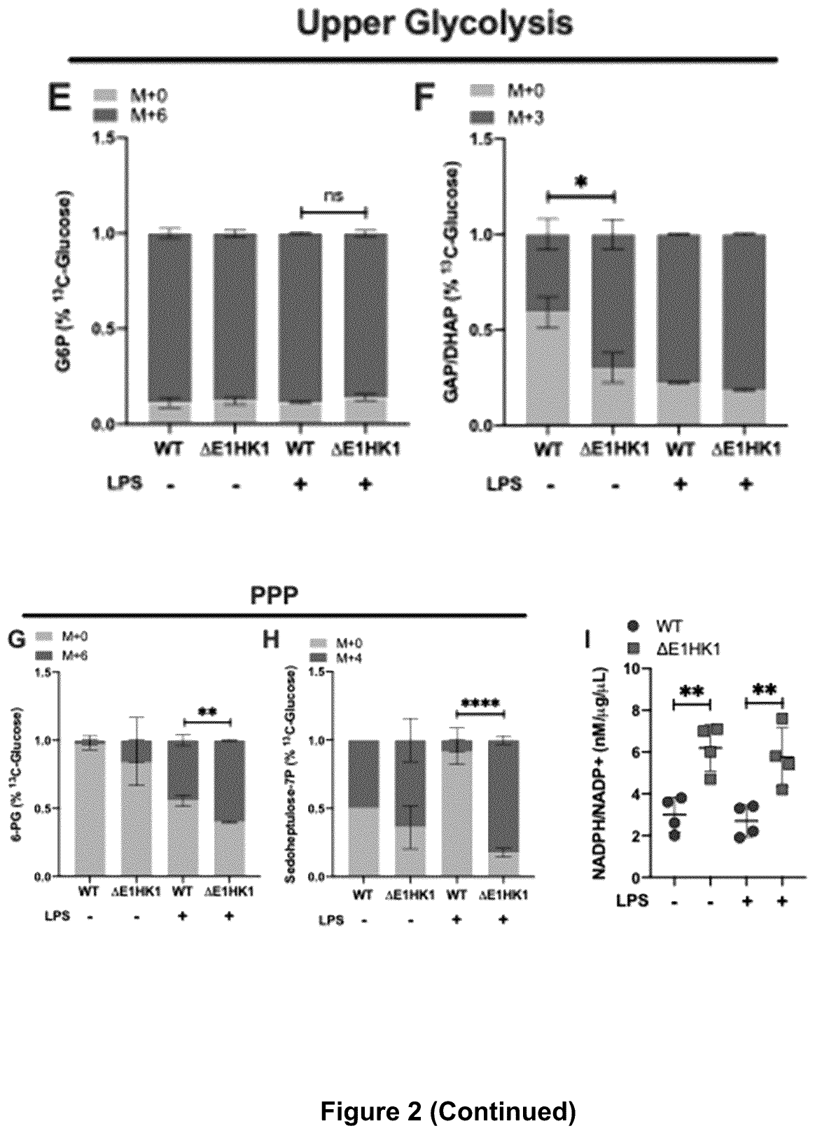

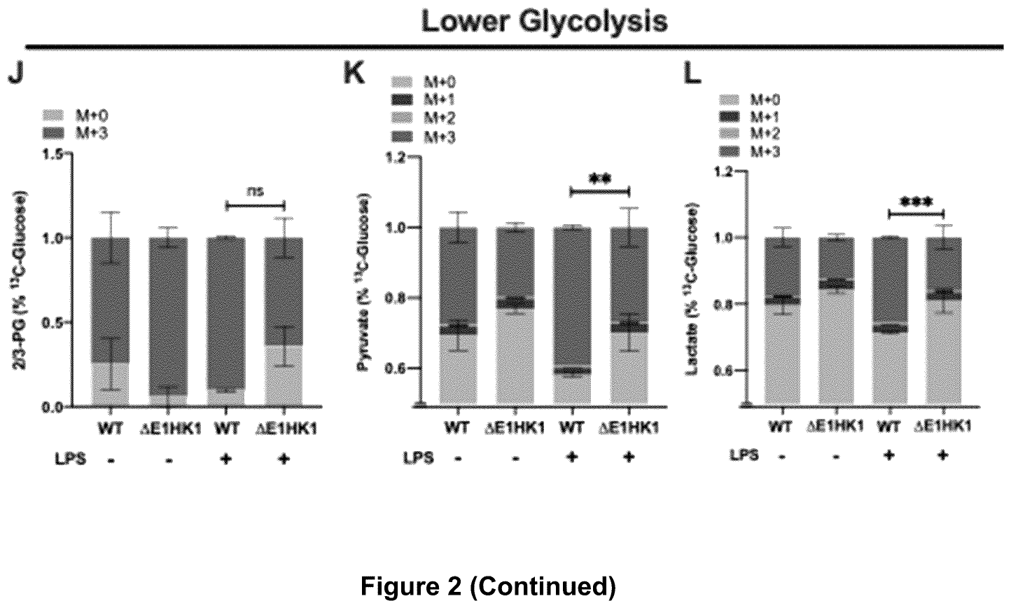

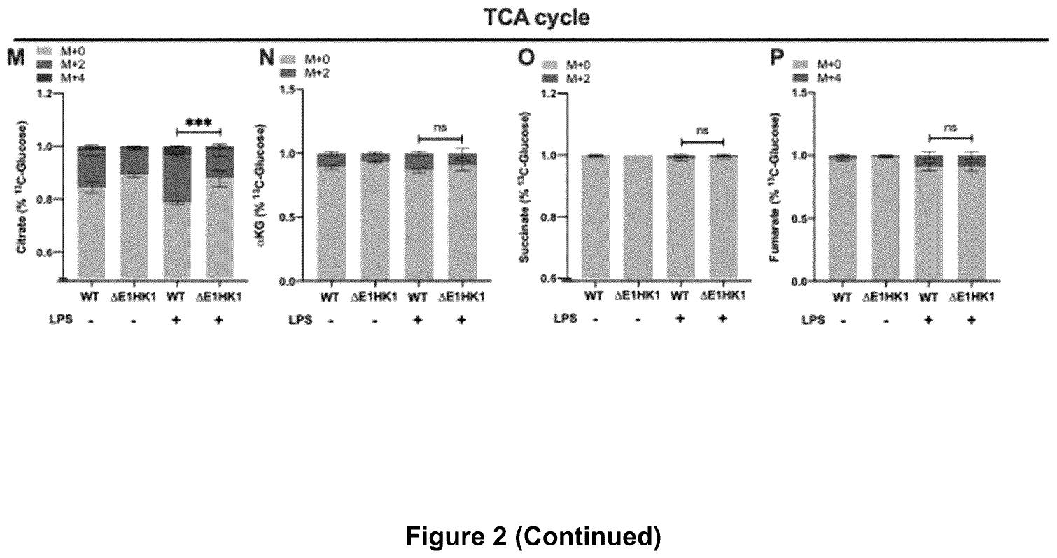

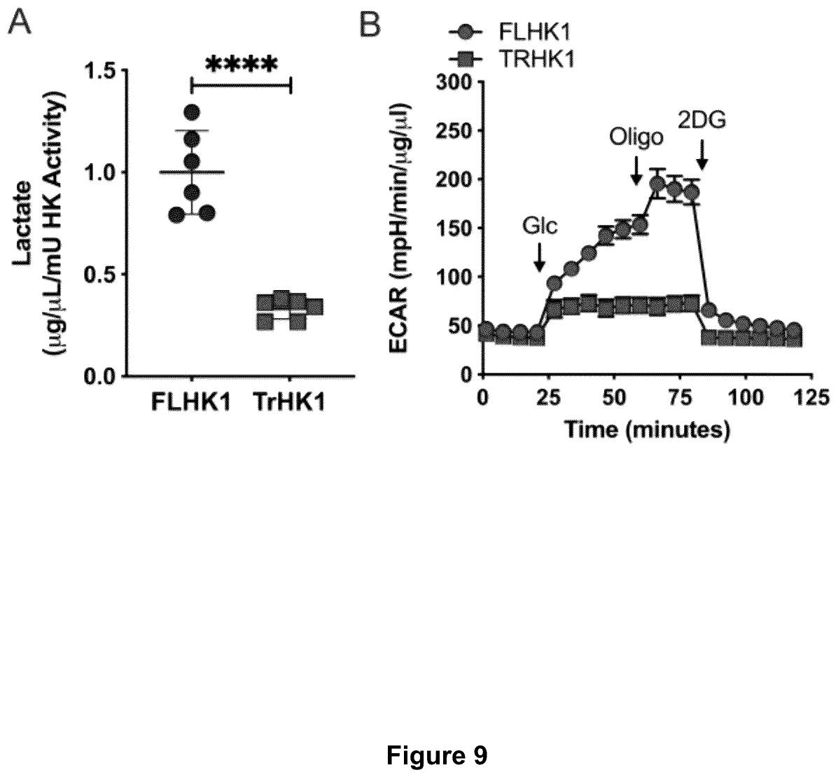

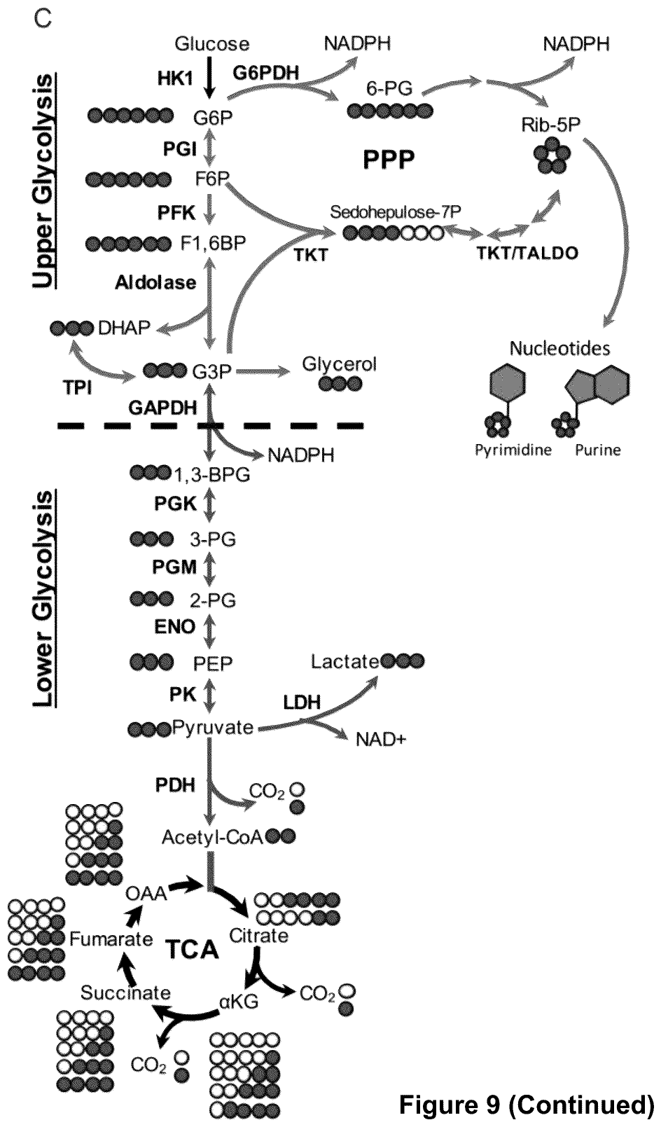

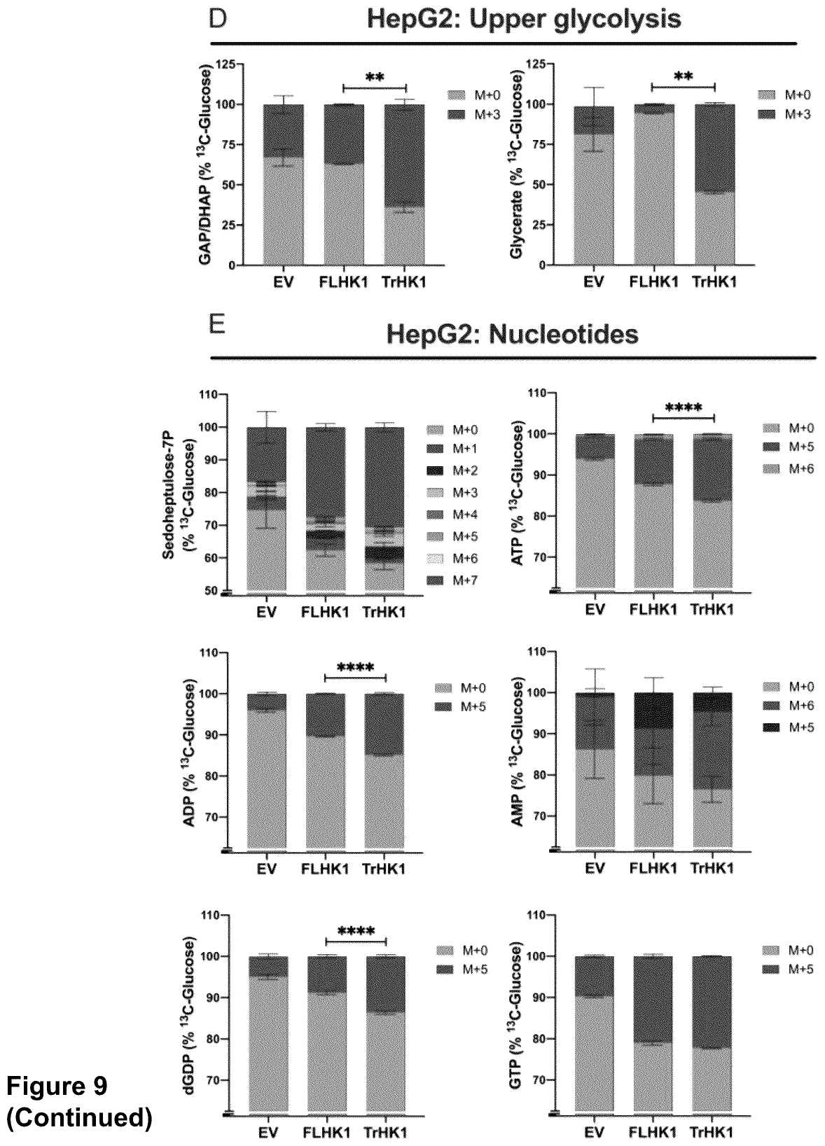

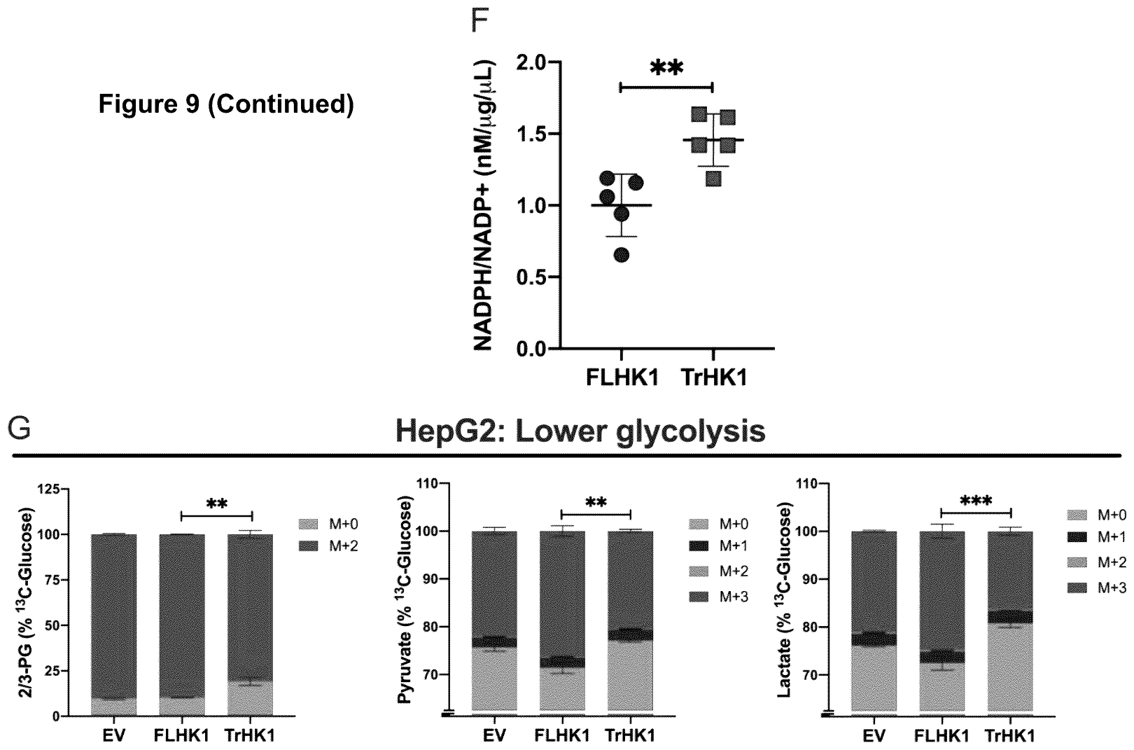

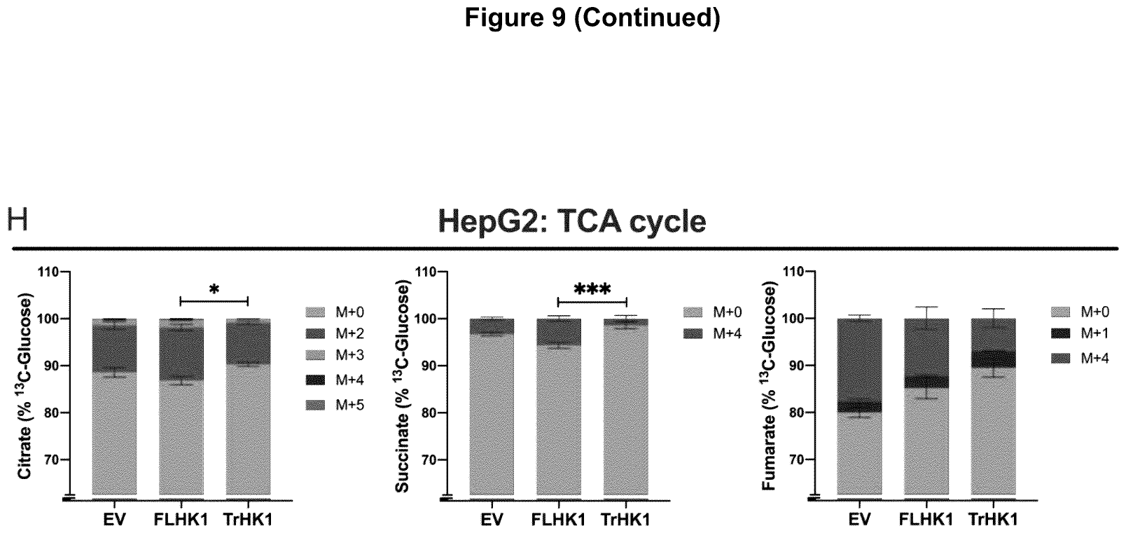

Since there was no difference in glucose phosphorylation with HK1 MBD deletion, we then assessed whether glucose metabolism was altered by the removal of the HK1 MBD. HepG2 cells expressing TrHK1 displayed reduced glycolysis, as assessed by extracellular lactate production and extracellular acidification rate (ECAR) (FIGS. 9A and 9B). Similarly, ΔE1HK1 BMDMs had significantly lower lactate production (FIG. 2A) and a significant reduction in ECAR upon immediate stimulation with LPS (FIG. 2B), which persisted up to at least 4 hours after LPS treatment (FIG. 2C). To further elucidate the mechanism responsible for the reduction in glycolysis observed in LPS-activated ΔE1HK1 BMDMs, we performed ¹³C₆-glucose tracing metabolomics in naïve and LPS-stimulated BMDMs derived from WT and ΔE1HK1 mice. We measured ¹³C-glucose incorporation through glycolysis, tricarboxylic acid (TCA) cycle, and PPP (FIG. 2D). While there was no difference in G6P levels between WT and ΔE1HK1 BMDMs (FIG. 2E), ΔE1HK1 BMDMs displayed higher ¹³C₆-glucose incorporation into glyceraldehyde-3-phosphate/dihydroxyacetone phosphate (GAP/DHAP) (FIG. 2F) and PPP intermediates 6-phosphogluconate (6-PG) and sedoheptulose-7-Phosphate after LPS activation (FIGS. 2G and 2H). Consistent with increased PPP metabolism, NADPH/NADP+ ratio was significantly increased in ΔE1HK1 BMDMs (FIG. 2I). Moreover, we observed decreased ¹³C₆-glucose incorporation of glycolytic intermediates below the level of GAPDH including 2/3-phosphoglycerate (2/3-PG), pyruvate, and lactate in LPS-treated BMDMs from ΔE1HK1 as compared to WT controls (FIG. 2J-L). The TCA cycle intermediate citrate showed decreased ¹³C₆-glucose incorporation (FIG. 2M), however, we did not observe a change in glucose-derived carbon incorporation into u-ketoglutarate (αKG), succinate, or fumarate between WT and ΔE1HK1 BMDMs (FIG. 2N-P). Since unlabeled glutamine can supply carbons into the TCA cycle through anaplerotic conversion to αKG, this may explain the lack of change in ¹³C₆-glucose labeling of TCA cycle metabolites downstream of citrate (Jha et al., 2015). These results are consistent with our earlier extracellular flux studies in HepG2 cells and BMDMs showing reduced glycolytic rate and extracellular lactate production with HK1 mitochondrial dissociation.

We also performed ¹³C₆-glucose labeling metabolomics in HepG2 cells with FLHK1 or TrHK1 overexpression and assessed similar metabolic pathways (FIG. 9C). In alignment with our results in BMDMs, there was a significant increase in glycolytic intermediates above the level of GAPDH (GAP and glycerate) (FIG. 9D), and an increase of ¹³C₆-glucose incorporation into the PPP intermediate sedoheptulose-7-P and several nucleotides in cells overexpressing TrHK1 (FIG. 9E), consistent with increased de novo nucleotide synthesis through the PPP. Additionally, we saw increased NADPH/NADP+ ratio (FIG. 9F), similar to BMDMs. Consistent with data obtained from BMDMs, we also found less ¹³C₆-glucose incorporation into lower glycolytic products such as 2/3-PG, pyruvate, and lactate (FIG. 9G) and TCA cycle intermediates citrate, succinate and fumarate (FIG. 9H) in cells overexpressing TrHK1 compared to FLHK1 expressing cells. Overall, these results indicate that dissociation of HK1 from mitochondria alters glycolysis and increases G6P incorporation into PPP at the expense of lower glycolysis (i.e., pyruvate and lactate).

### Constitutive HK1 Mitochondrial Dissociation Increases Inflammatory Cytokine Production In Vitro & In Vivo

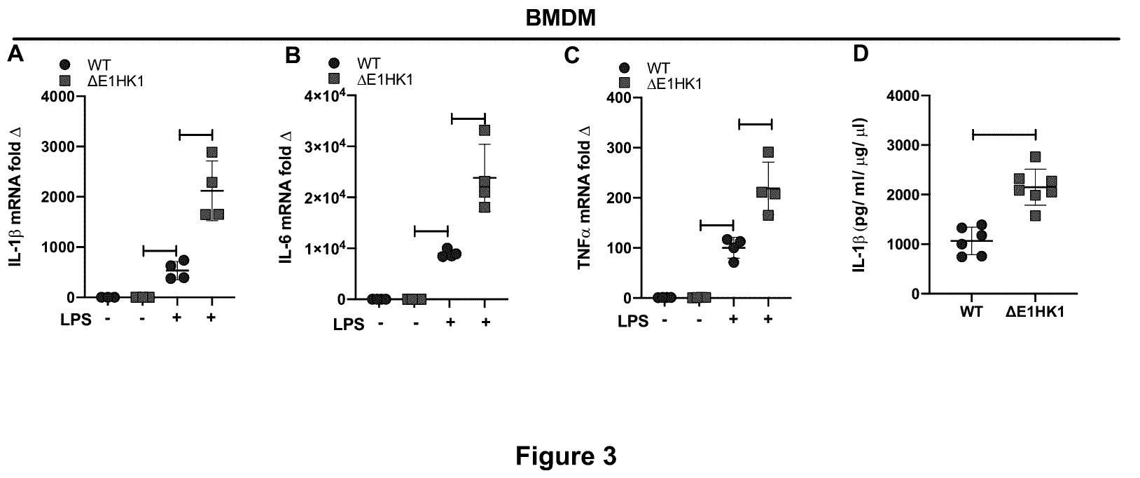

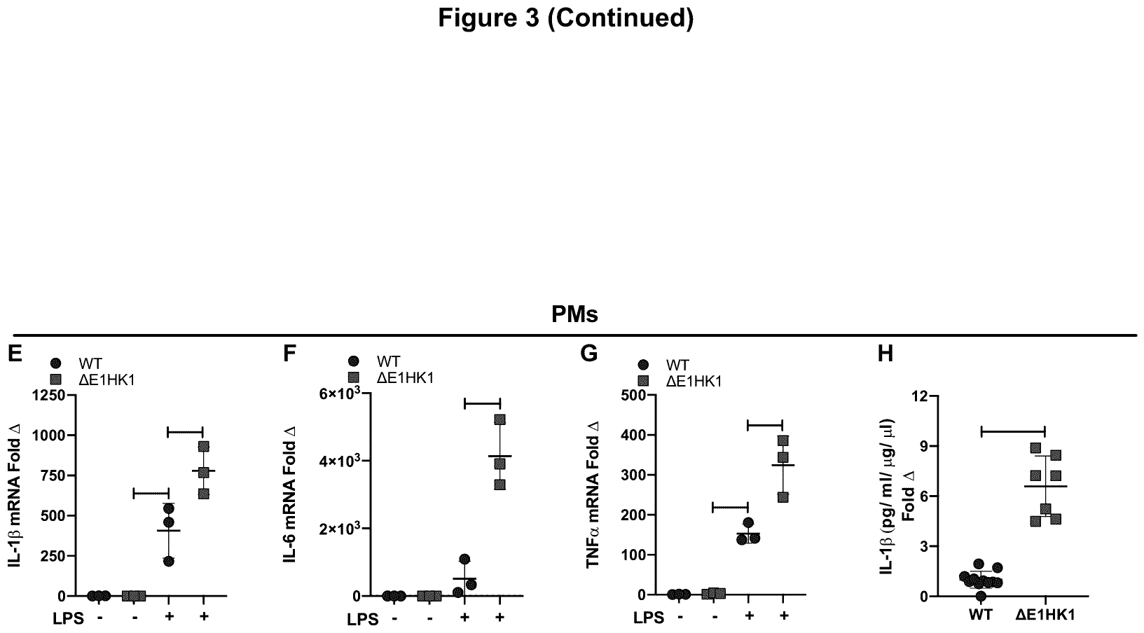

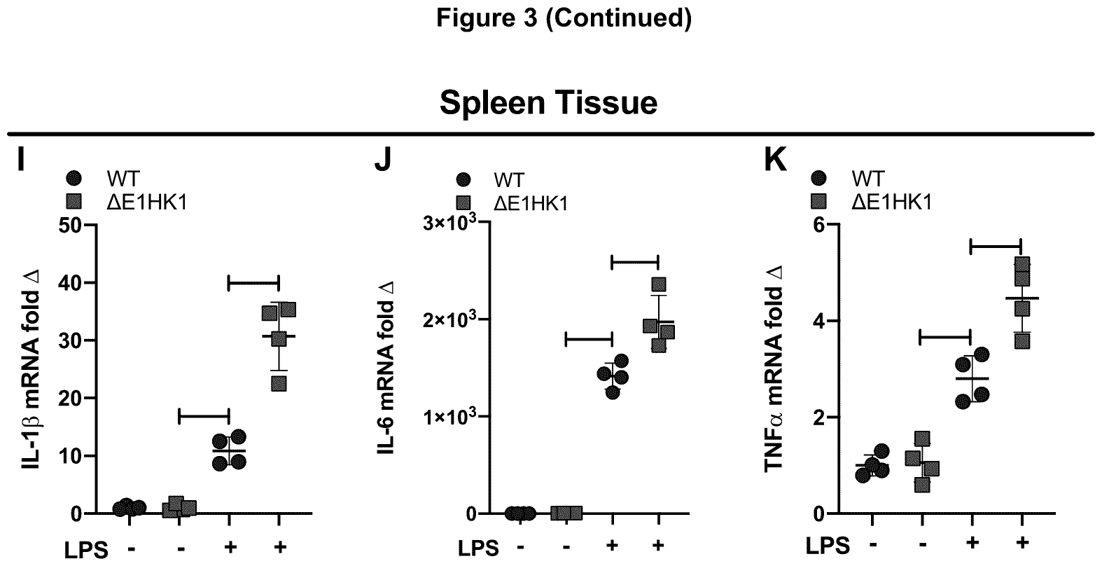

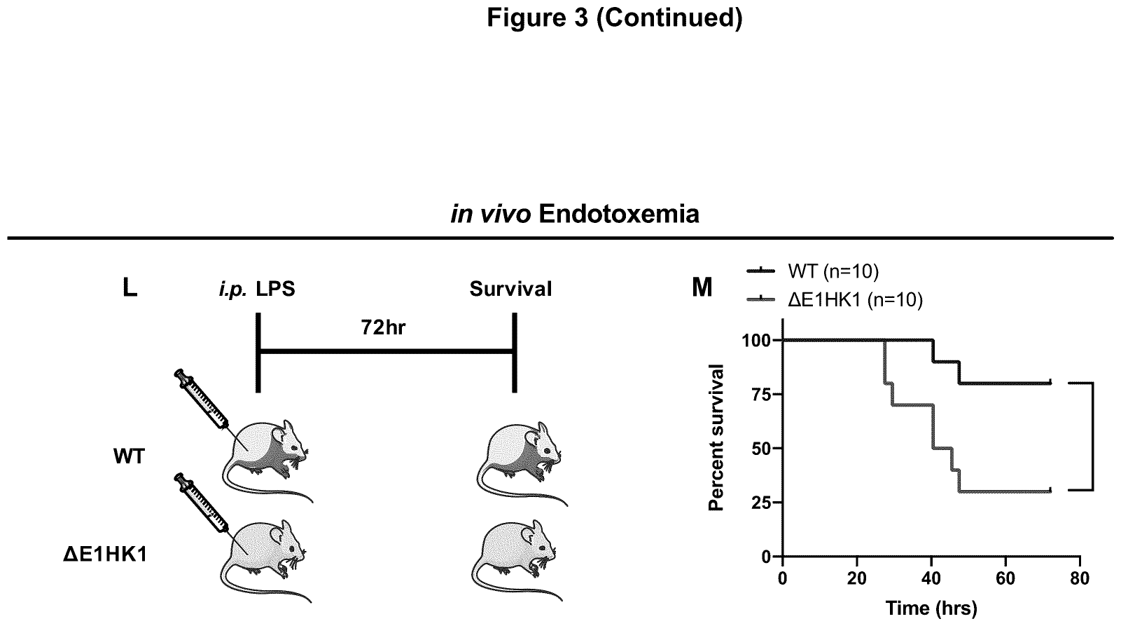

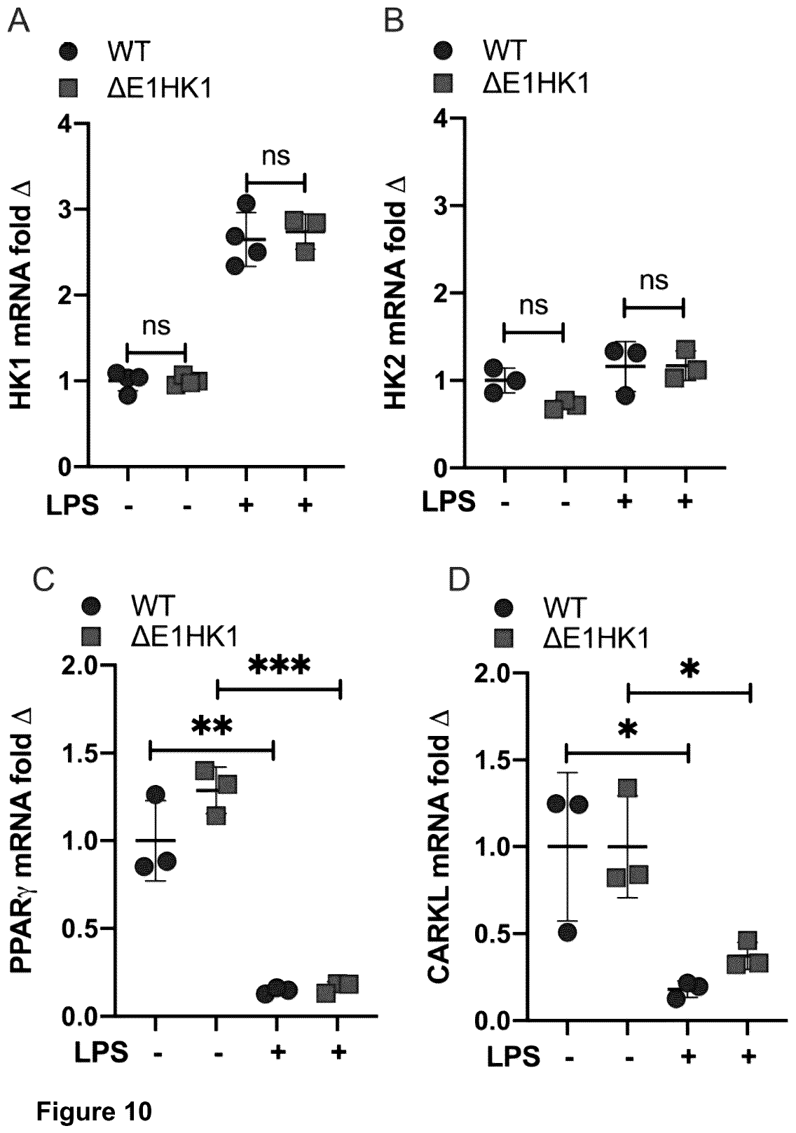

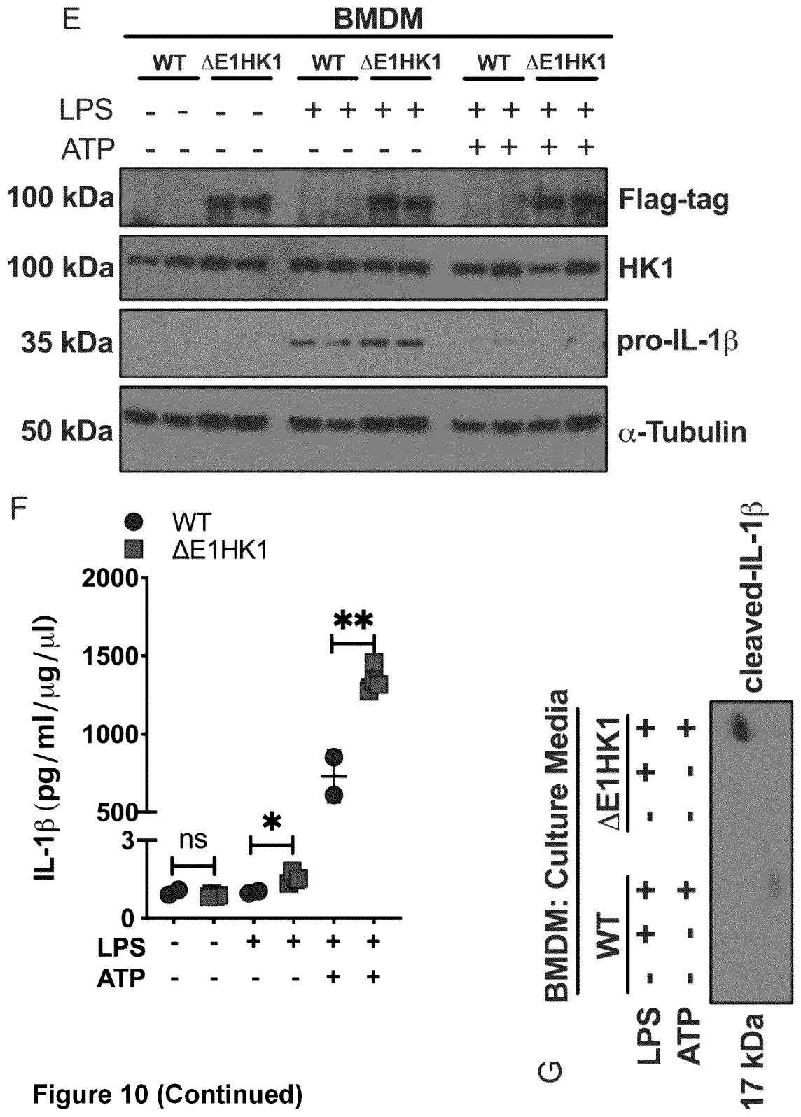

Glycolysis serves a vital role in the initiation and maintenance of proper effector function of activated macrophages (Rodríguez-Prados et al., 2010; Pearce and Pearce, 2013; Everts et al., 2014). PPP metabolism has been shown to be upregulated in LPS activated macrophages and is necessary for proper inflammatory activation (Haschemi et al., 2012). Overexpression of G6PD, the rate-limiting enzyme of the oxidative-PPP, in macrophages was shown to enhance pro-inflammatory cytokine production (Ham et al., 2013). Furthermore, the non-oxidative branch of the PPP was shown to be highly upregulated in LPS activated macrophages, which is thought to be necessary for providing de novo nucleotides to support their characteristic transcriptional response (Martinez et al., 2006; Nagy and Haschemi, 2015). Given the metabolic alterations seen with disruption of HK1 mitochondrial binding, we next assessed the consequence of these changes on the effector function of LPS-activated macrophages. We noted a significant increase in inflammatory cytokines IL-1β, IL-6 and TNFα mRNA expression in BMDMs from ΔE1HK1 mice compared to WT mice in response to LPS (FIG. 3A-3C). In addition, BMDMs from ΔE1HK1 mice displayed increased IL-1β protein production after LPS treatment (FIG. 3D). As expected, LPS-activated BMDMs from ΔE1HK1 and WT mice showed a similar increase in mRNA expression of HK1, while there was no change in the levels of HK2 mRNA (FIGS. 10A and 10B). We also assessed mRNA levels of anti-inflammatory cytokines peroxisome proliferator-activated receptor gamma (PPARγ) and carbohydrate kinase-like protein (CARKL) in BMDMs, and found no difference in their expression between ΔE1HK1 and WT cells (FIGS. 10C and 10D). Intracellular protein expression of pro-IL-1β (molecular weight ˜35 kDa) in control and LPS-treated BMDMs showed an increase in ΔE1HK1 compared to WT cells, and addition of ATP induced cleavage and secretion of IL-1β in both groups (FIG. 10E). Moreover, using extracellular media from BMDMs treated with LPS+ATP, we demonstrate that IL-1β protein secretion increased in ΔE1HK1 BMDMs compared to WT controls (FIGS. 10F and 10G).

PMs isolated from ΔE1HK1 mice also showed increased IL-1β, IL-6 and TNFα mRNA expression relative to WT cells when stimulated with LPS, similar to BMDMs (FIG. 3E-3G). PMs from ΔE1HK1 mice also had increased IL-1β protein secretion after LPS treatment, as assessed by ELISA (FIG. 3H). Furthermore, we extracted mRNA from the spleens of mice treated with LPS or PBS for 4 hours, and found a significant increase in IL-1β, IL-6, and TNFα mRNA in spleens from ΔE1HK1 mice compared to WT mice treated with LPS (FIG. 3I-K). Lastly, we performed an in vivo LPS-induced endotoxemia survival experiment in these mice (FIG. 3L). Three-days after sub-lethal LPS administration, ΔE1HK1 mice had significantly reduced survival compared to littermate controls (FIG. 3M). Collectively, these results indicate that lack of HK1 mitochondrial binding increases inflammatory cytokine production and impairs survival in response to LPS-induced endotoxemia.

### Inhibition of PPP Reverses Hyper-Inflammation Induced by HK1 Mitochondrial Dissociation

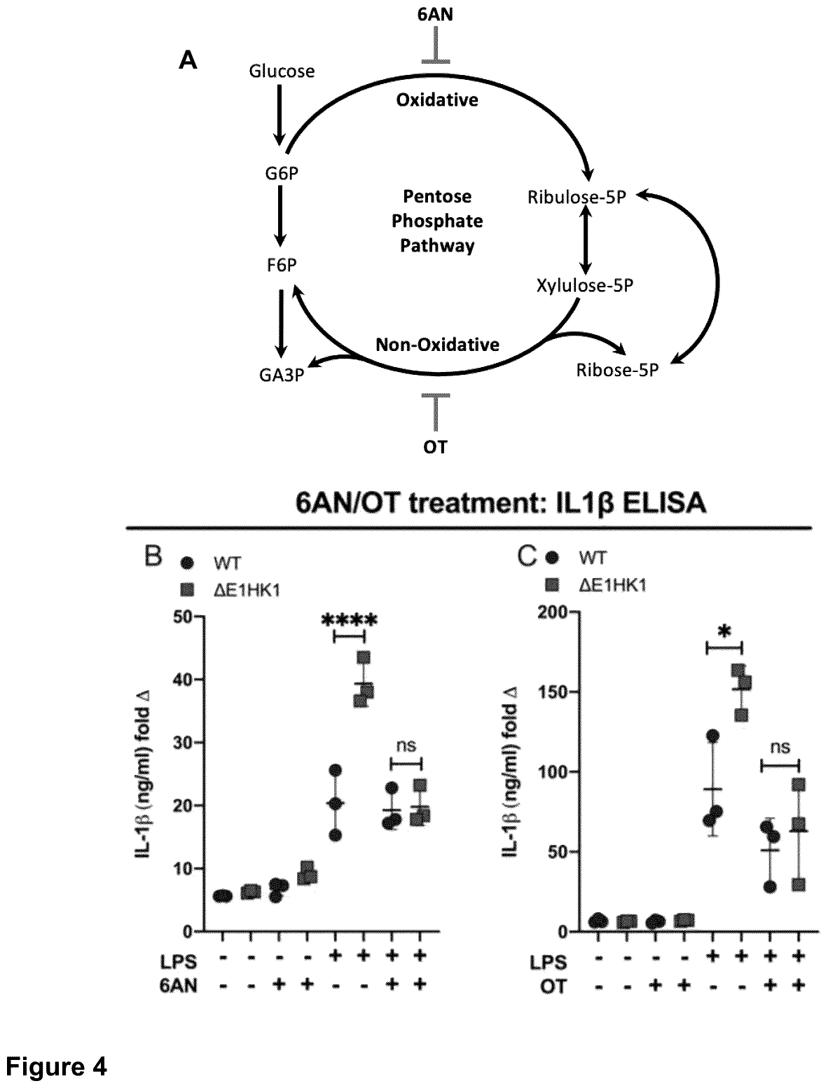

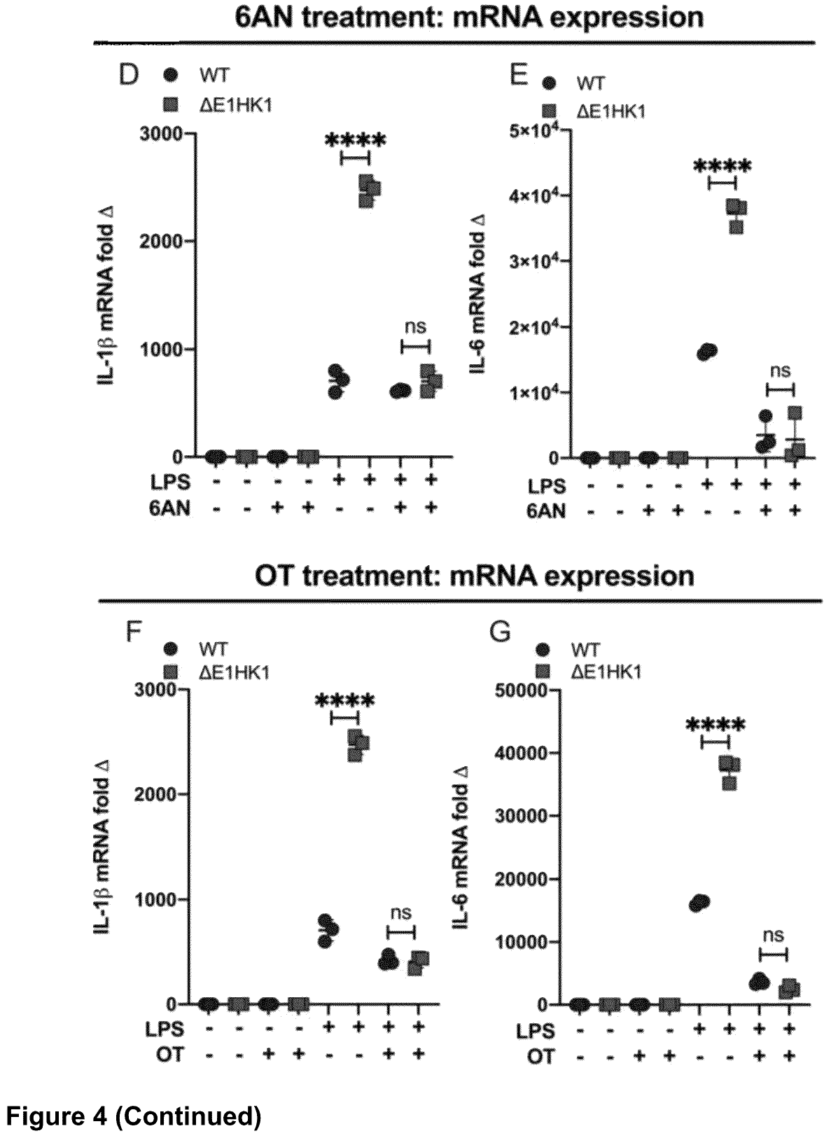

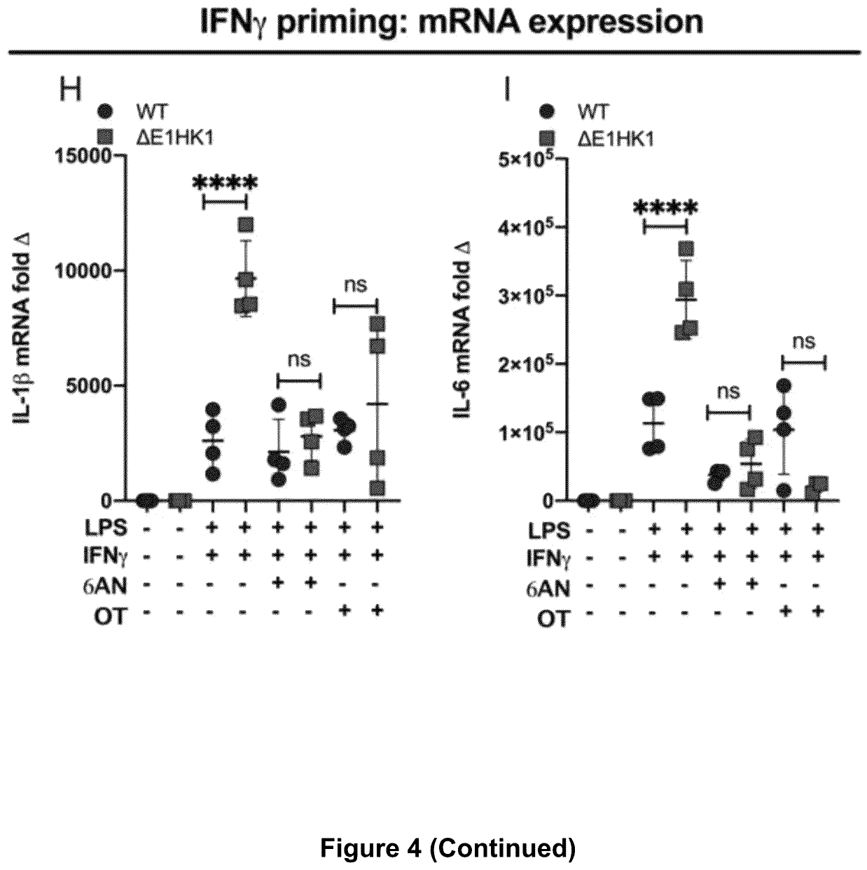

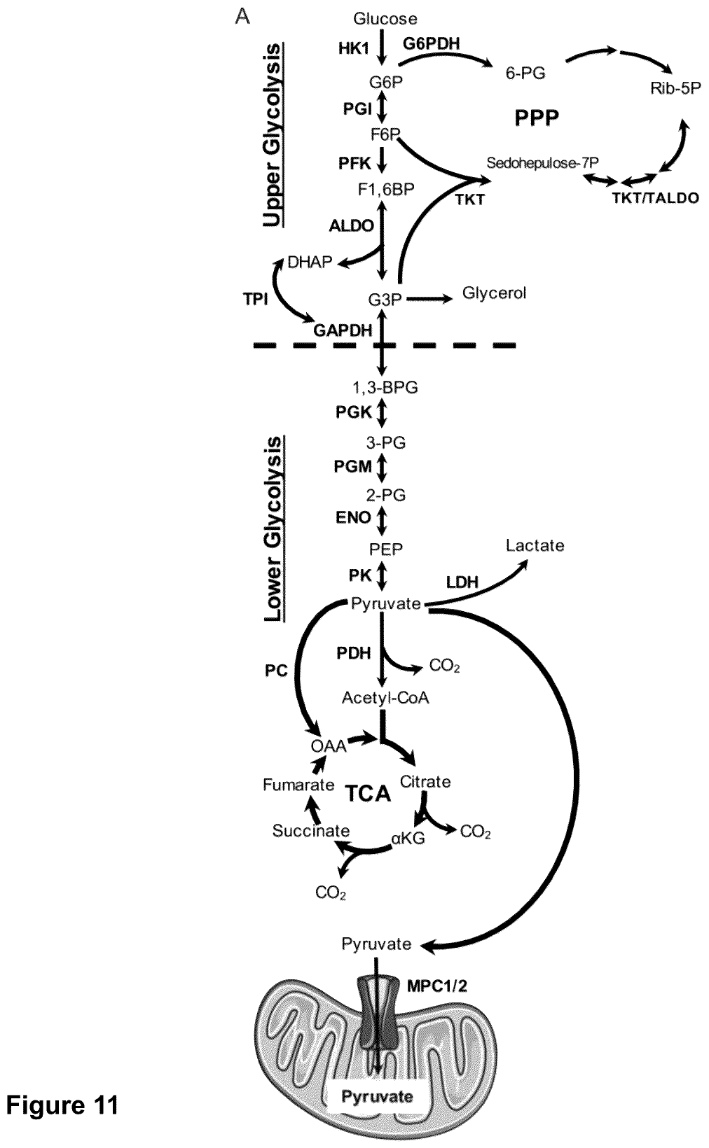

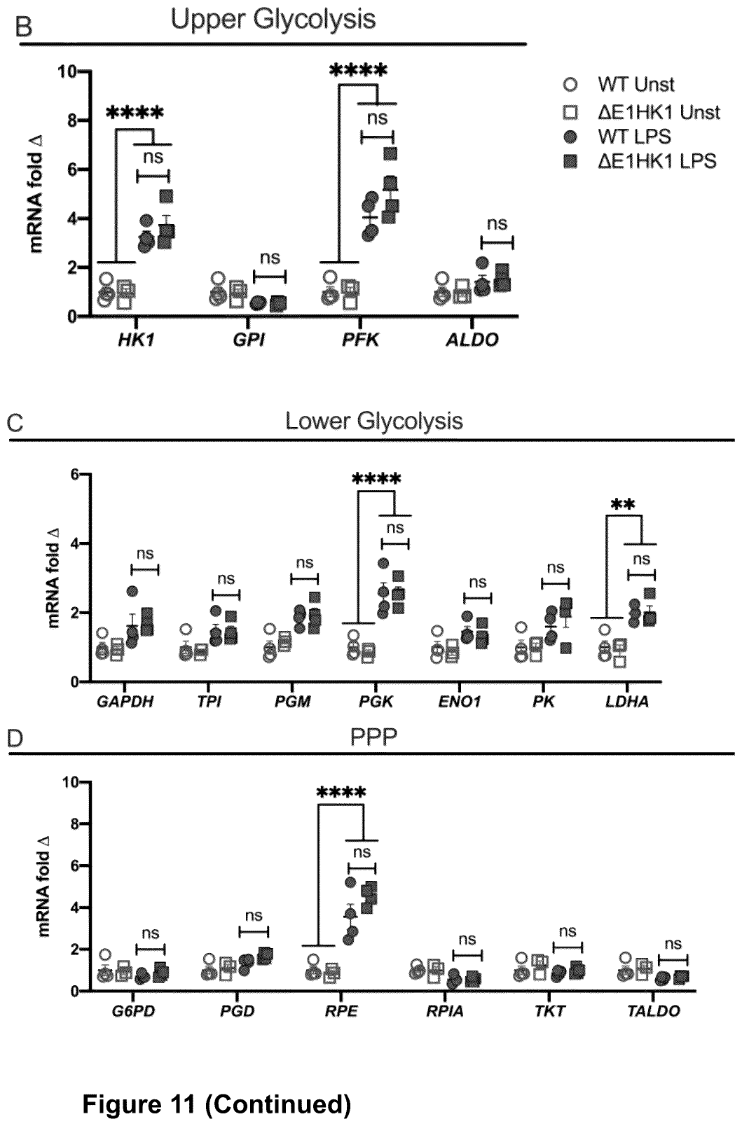

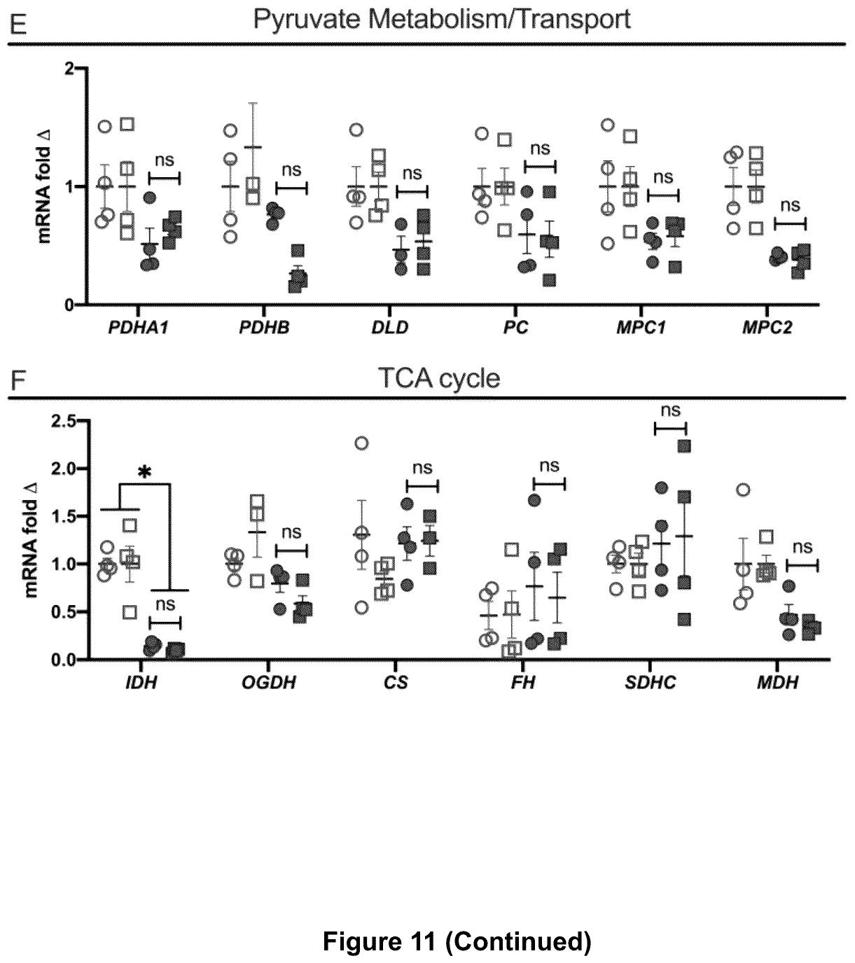

The PPP is upregulated in pro-inflammatory (M1) macrophages and neutrophils, and reduction of PPP metabolites reduces M1 macrophage cytokine production (Nagy and Haschemi, 2015; Baardman et al., 2018). Thus, to determine whether the hyper-inflammatory response seen in ΔE1HK1 mice is dependent on the increased PPP metabolism, we used two methods to inhibit the PPP in BMDMs from these mice: 1) 6-aminonicotinamide (6AN), which is metabolized into an NADP⁺ analog and competitively inhibits NADPH-producing enzymes 6-phosphogluconate dehydrogenase (PGD) and glucose 6-phosphate dehydrogenase (G6PD), and 2) oxythiamine (OT), a thiamine antagonist, which suppresses the non-oxidative synthesis of ribose by inhibiting the transketolase (TKT) enzyme (FIG. 4A) (Tyson et al., 2000; Wang et al., 2013). Treatment of BMDMs from ΔE1HK1 mice with LPS caused a higher release of IL-1β protein, which was reversed with either 6AN or OT (FIGS. 4B and 4C). The increase in IL-1β and IL-6 mRNA levels in BMDMs of ΔE1HK1 mice was also abolished when treated with either 6-AN (FIGS. 4D and 4E) or OT (FIGS. 4F and 4G). To further validate these results, we primed BMDMs with interferon-gamma (IFN-γ), followed by LPS treatment and addition of either 6AN or OT and measurement of IL-1β and IL-6 mRNA. The increase in IL-1β and IL-6 mRNA levels in BMDMs of ΔE1HK1 mice were rescued with 6AN and OT treatment in IFN-γ primed BMDMs (FIGS. 4H and 4I). Additionally, we show that there was no significant difference in the mRNA expression of enzymes involved in glycolysis, PPP, pyruvate transport, or TCA cycle metabolism with and without LPS treatment of BMDMs between WT and ΔE1HK1 mice (FIG. 1A-11F), indicating that the mechanism for the glycolytic block causing increased PPP metabolism is not at the transcriptional level. Together, these studies imply that the mechanism by which HK1 dislocation from mitochondria causes an increase in cytokine production is through increased PPP metabolism.

GAPDH Activity is Attenuated in Macrophages with HK1 Mitochondrial Detachment

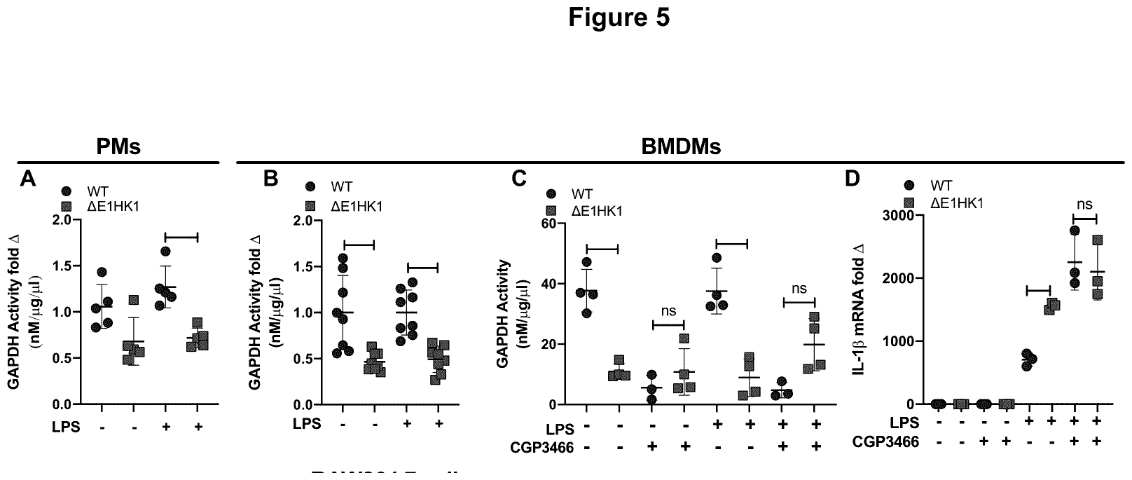

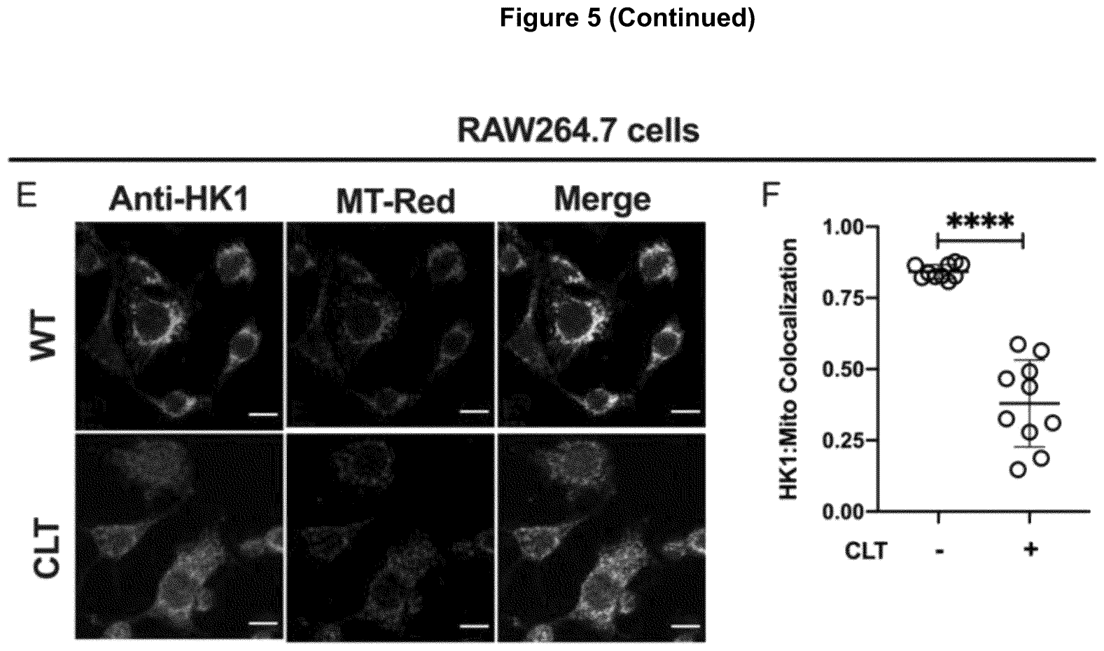

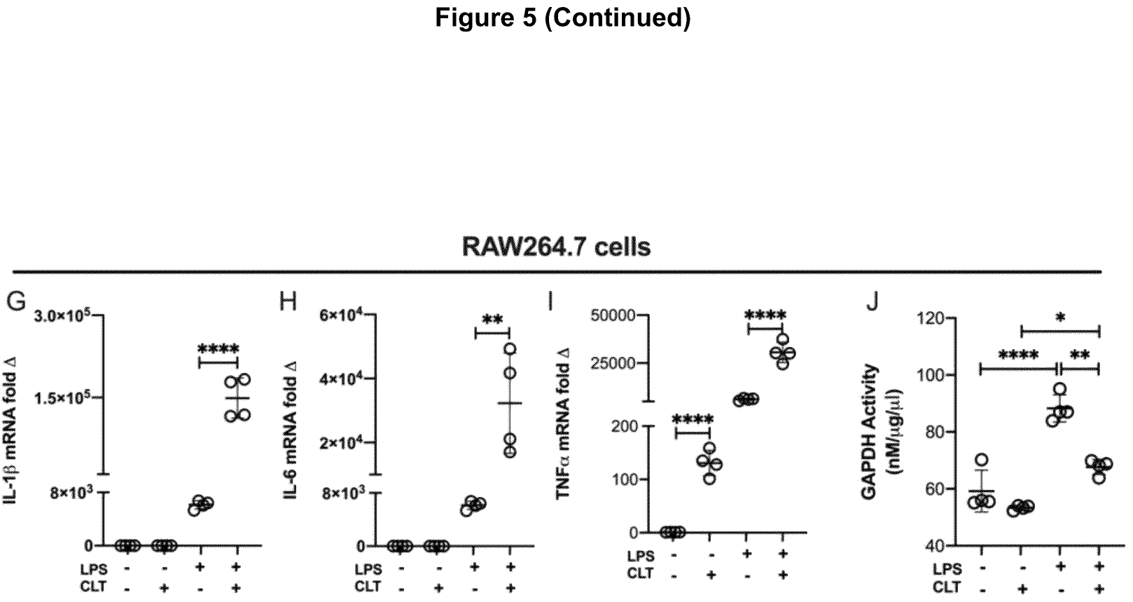

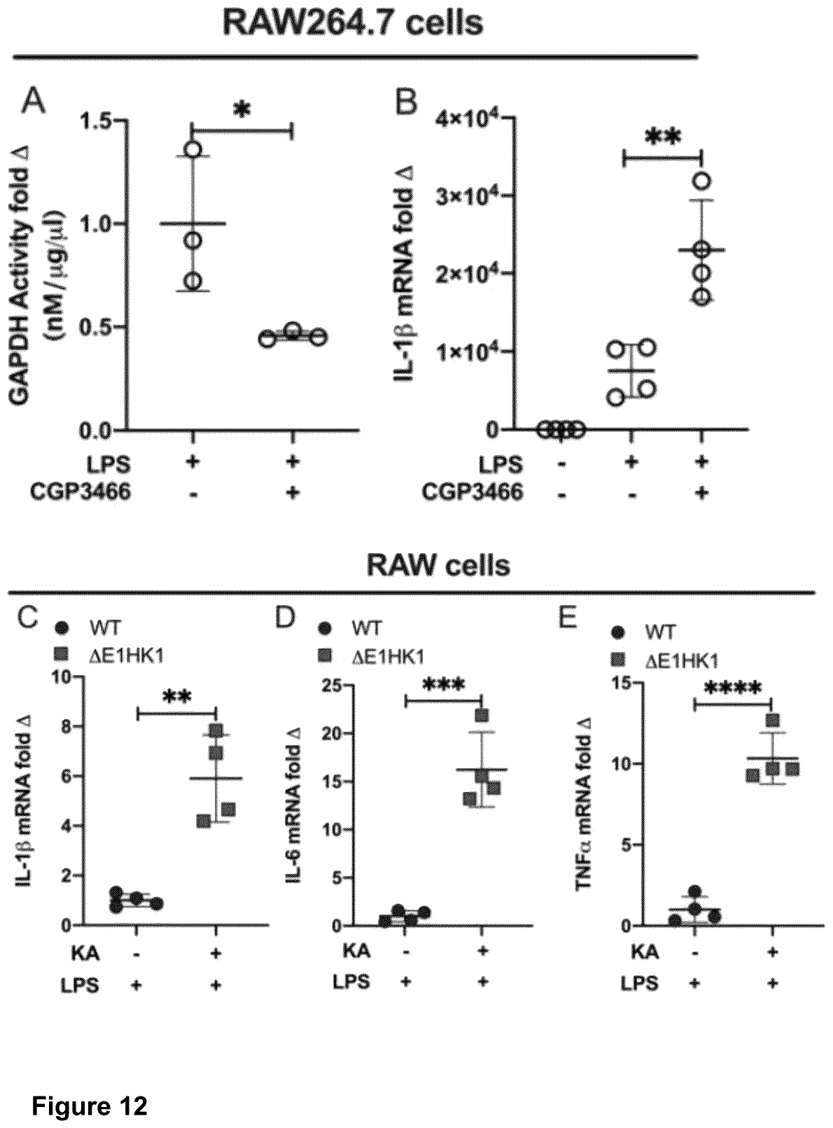

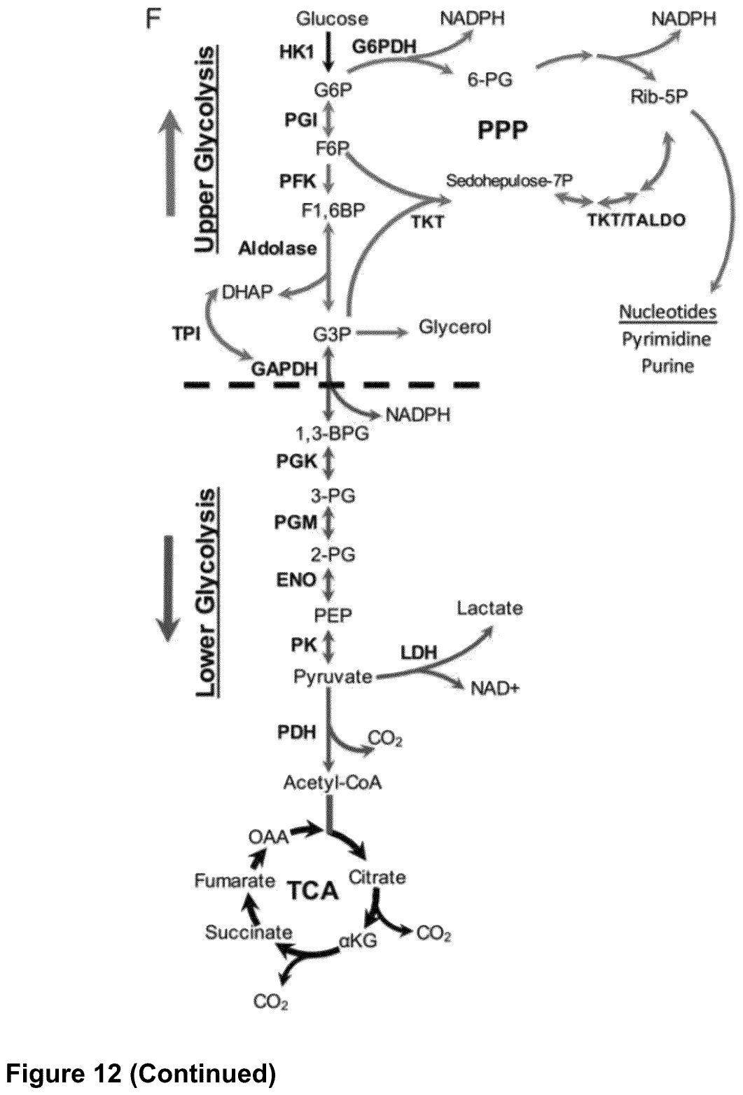

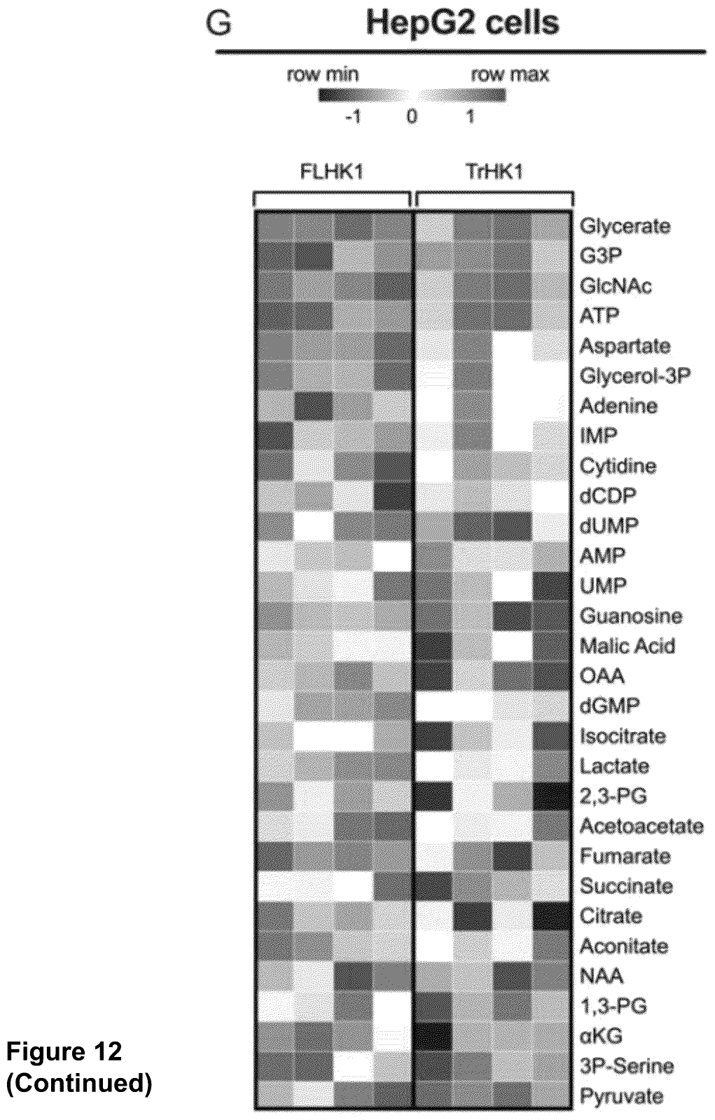

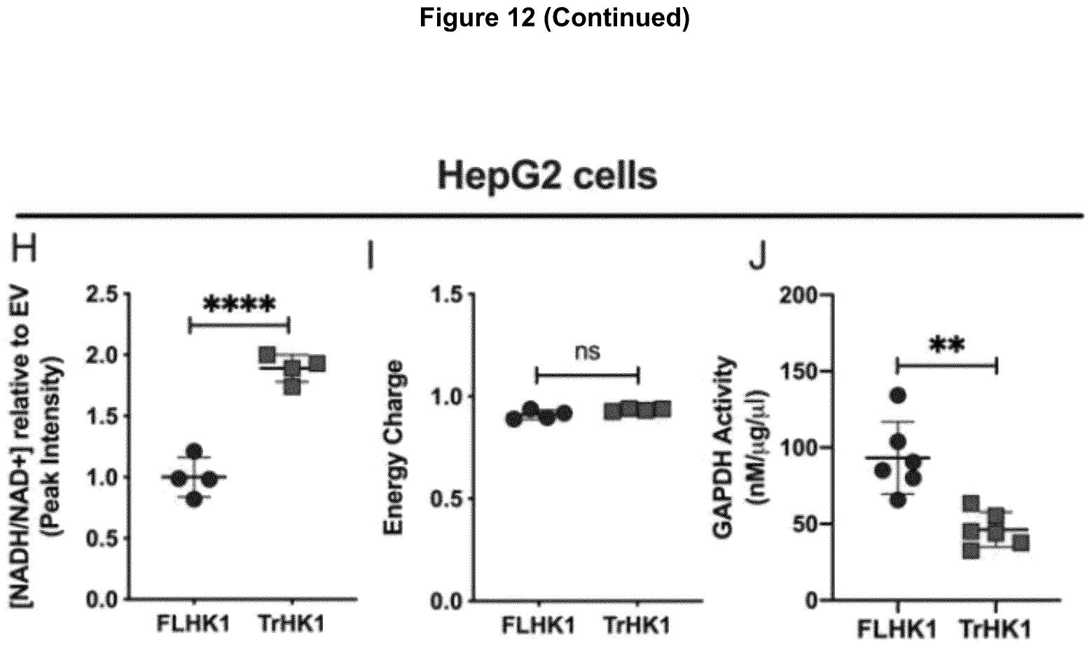

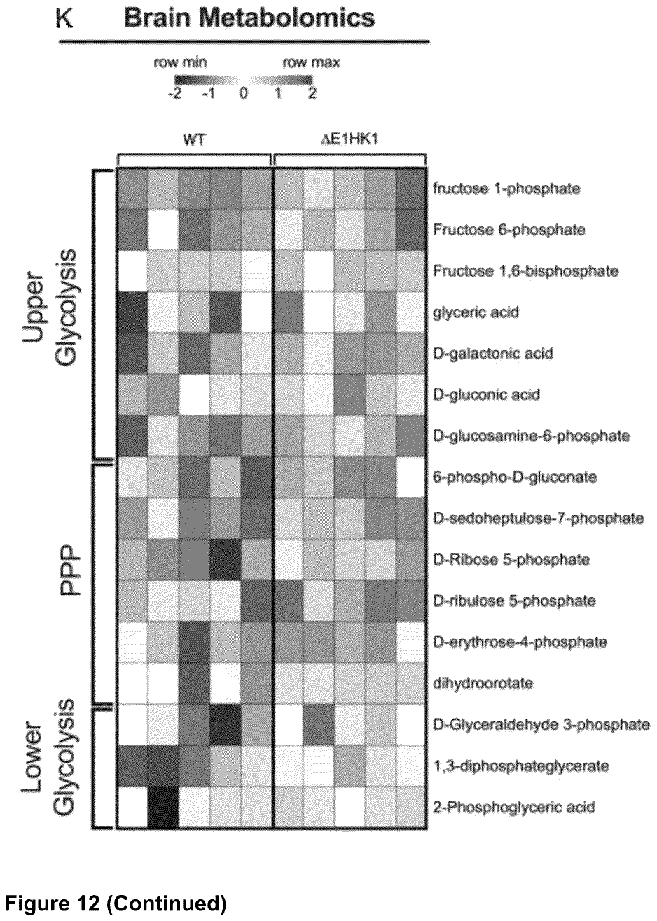

We next studied the mechanism by which cytosolic HK1 reduces glycolysis and shifts G6P towards the PPP. Analysis of the ¹³C₆-glucose labeling metabolomics indicated an increase in metabolites in upper glycolysis (i.e., metabolites upstream of the GAPDH-mediated step in glycolysis) and a reduction in metabolites in lower glycolysis (i.e., downstream of GAPDH), suggesting a block in glycolysis at the level of GAPDH. As expected PMs and BMDMs from ΔE1HK1 mice displayed significantly decreased GAPDH activity at baseline and with LPS treatment (FIGS. 5A and 5B). The difference in GAPDH activity of BMDMs between WT and ΔE1HK1 mice was abolished by the GAPDH inhibitor, CGP3466 (FIG. 5C). CGP3466 also increased IL-1β mRNA expression in WT BMDMs to the level of the hyper-inflamed ΔE1HK1 BMDMs (FIG. 5D). Moreover, in RAW264.7 cells, GAPDH activity was decreased with the addition of CGP3466 in the presence of LPS (FIG. 12A), and the increase in IL-1β mRNA with LPS was amplified with the addition of CGP3466 (FIG. 12B). Additionally, treatment of RAW264.7 cells with koningic acid (KA), another potent inhibitor of GAPDH, resulted in increased IL-1β, IL-6 and TNFα mRNA expression after LPS treatment (FIG. 12C-E).

We also performed steady-state metabolomics in HepG2 cells with TrHK1 overexpression and observed a similar metabolic block at the level of GAPDH (FIGS. 12F and 12G). While there was an increase in the reductive state of the cell (as assessed by the measurement of NADH/NAD+ levels) with overexpression of TrHK1 (FIG. 12H), the energy charge was not different between FLHK1 and TrHK1 overexpression cells (FIG. 12I). Consistent with findings in BMDMs, overexpression of TrHK1 was associated with a significant decrease of GAPDH activity (FIG. 12J). Finally, we performed unbiased metabolomics in whole brain isolates, which is a tissue known to have high HK1 expression. Whole brain steady-state metabolomics revealed increased PPP metabolites (i.e., sedoheptulose 7-phosphate, 6-PG, ribose-5-phosphate, GAP, and erythrose 4-phosphate) in tissues from ΔE1HK1 mice compared to WT controls, further validating the results from BMDMs and HepG2 cells (FIG. 12K). Together, these data indicate that HK1 mitochondrial binding is an important regulator of GAPDH function, and that dislocation of HK1 from the mitochondria causes an inhibition of GAPDH activity.

We next assessed the effects of acute dislocation of HK1 from the mitochondria (as opposed to constitutive dislocation of the protein, as occurs in the ΔE1HK1 mice) on GAPDH activity. Clotrimazole (CLT), an antifungal drug, is known to dislocate HKs from the mitochondria (Huang et al., 2002; Shoshan-Barmatz et al., 2010; Sen et al., 2015). As expected, CLT caused a significant increase in HK1 dislocation from the mitochondria in WT BMDMs (FIGS. 5E and 5F). Despite minimal change in cytokine mRNA levels with CLT at baseline, stimulation with LPS induced a significant increase in the mRNAs of IL-1β, IL-6 and TNFα in the CLT-treated cells as compared to vehicle treated controls (FIG. 5G-I). Finally, CLT also caused a significant decrease in GAPDH activity in RAW264.7 cells after LPS treatment (FIG. 5J). These results indicate that both acute and constitutive displacement of HK1 from the mitochondria cause a significant reduction in GAPDH activity and accentuates inflammatory cytokine production after LPS treatment.

### Cytosolic HK1 Mediates GAPDH Nitrosylation Through S100A8/9 Binding

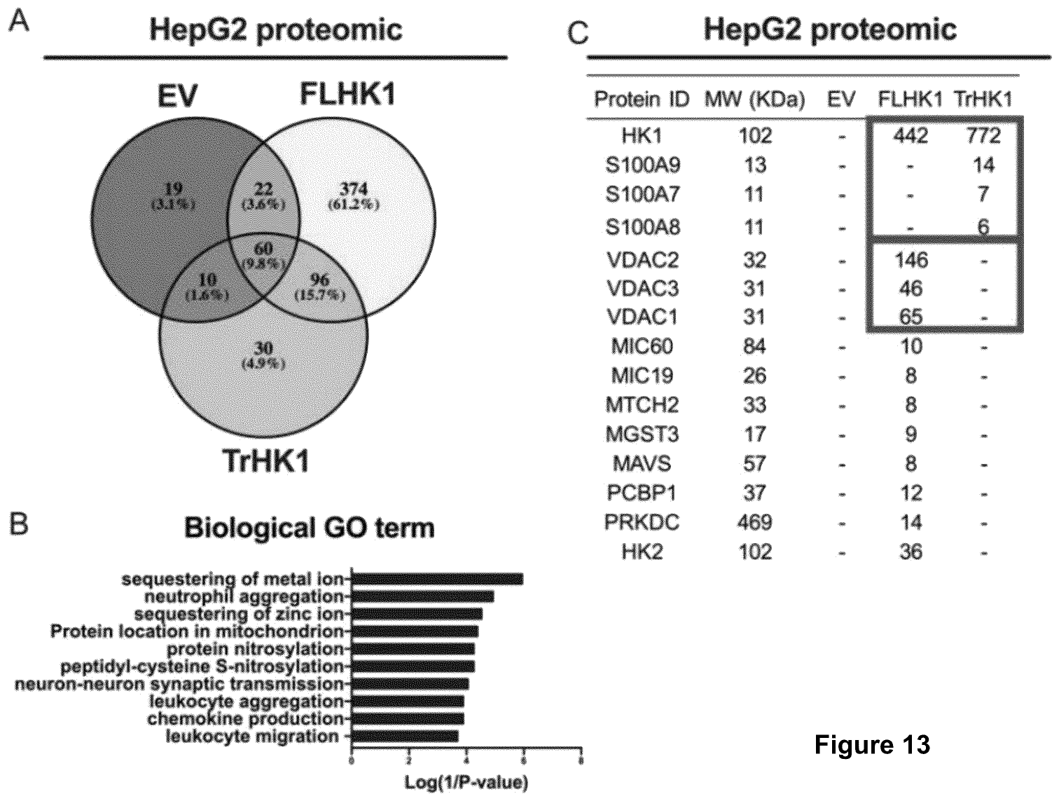

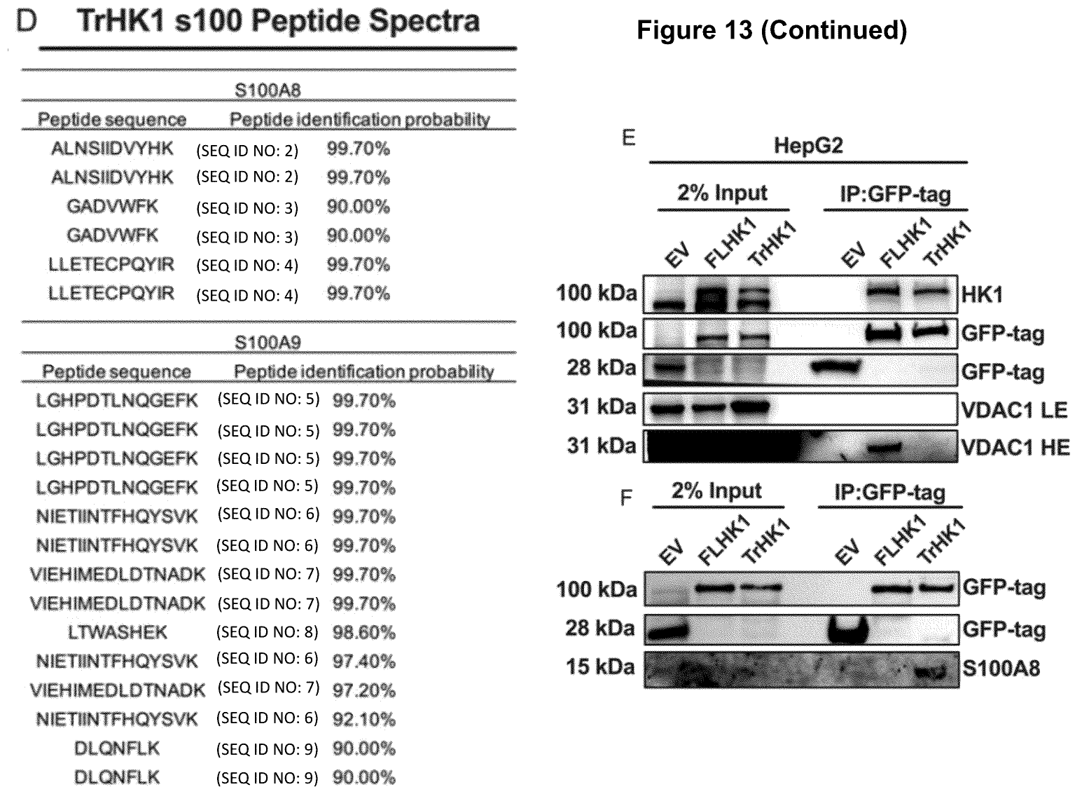

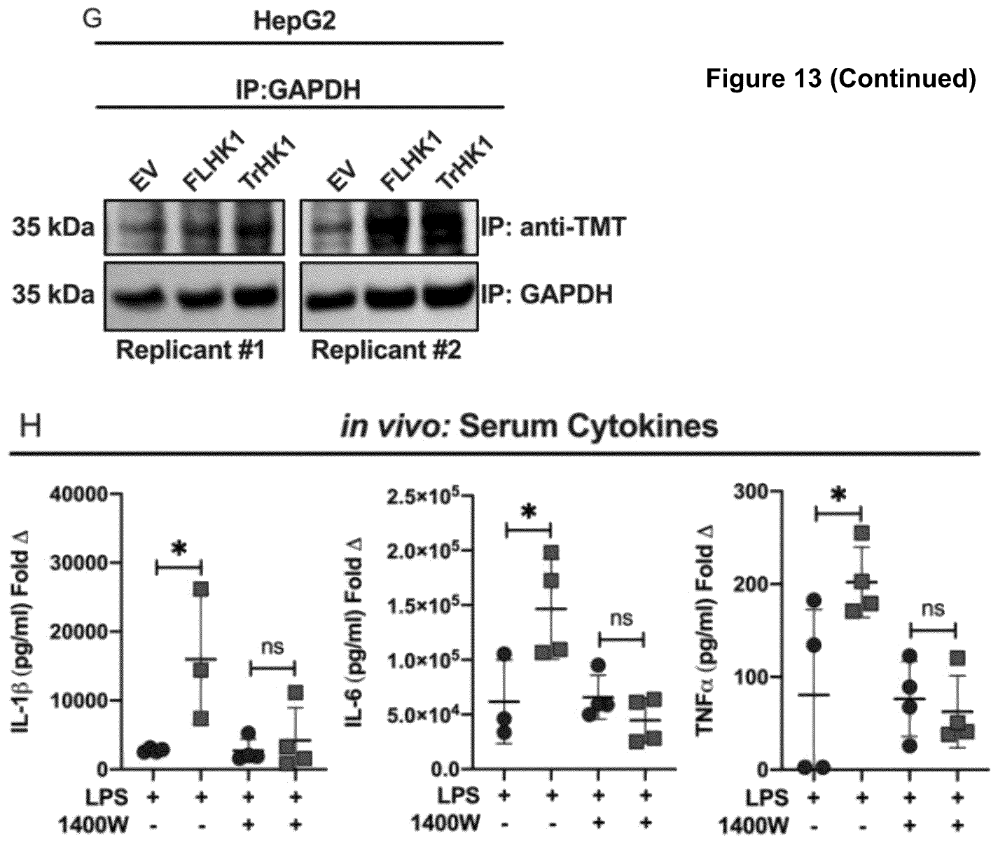

We next studied the mechanism of GAPDH inactivation with HK1 mitochondrial dissociation. We hypothesized that cytosolic HK1 binds to a distinct set of proteins compared to the mitochondrial bound HK1. To test this, we performed an unbiased immunoprecipitation (IP) experiment followed by mass spectrometry (MS) in HepG2 cells with overexpression of FLHK1 and TrHK1. Proteomic analysis yielded 611 unique spectra which corresponded to 175 identified proteins (FIG. 13A). Biological gene-ontology (GO) term analysis revealed several differences between the two treatments, as depicted in FIG. 13B (Mi et al., 2019). VDAC proteins (VDAC1, VDAC2, and VDAC3) were enriched in the FLHK1 pulldown, which is expected since HK1 in its native form binds to these proteins on the OMM (FIG. 13C) (Shoshan-Barmatz et al., 2009; Zhang et al., 2016). Among proteins enriched in cells overexpressing TrHK1, there was an over-representation of the S100 family of proteins, of specific interest were S100A8 and S100A9, which are known to have inflammatory functions (FIG. 13D) (Wang et al., 2018). We confirmed that S100A8 binds strongly to the truncated HK1 via co-IP in HepG2 cells overexpressing either FLHK1 or TrHK1 (FIGS. 13E and 13F).

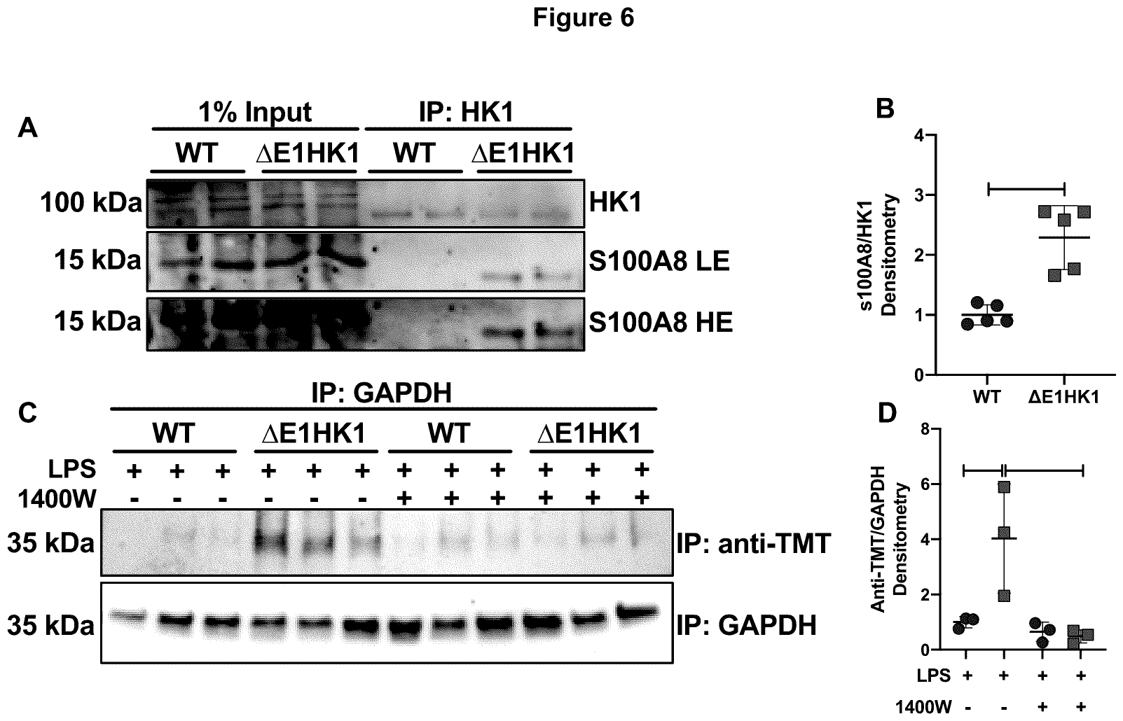

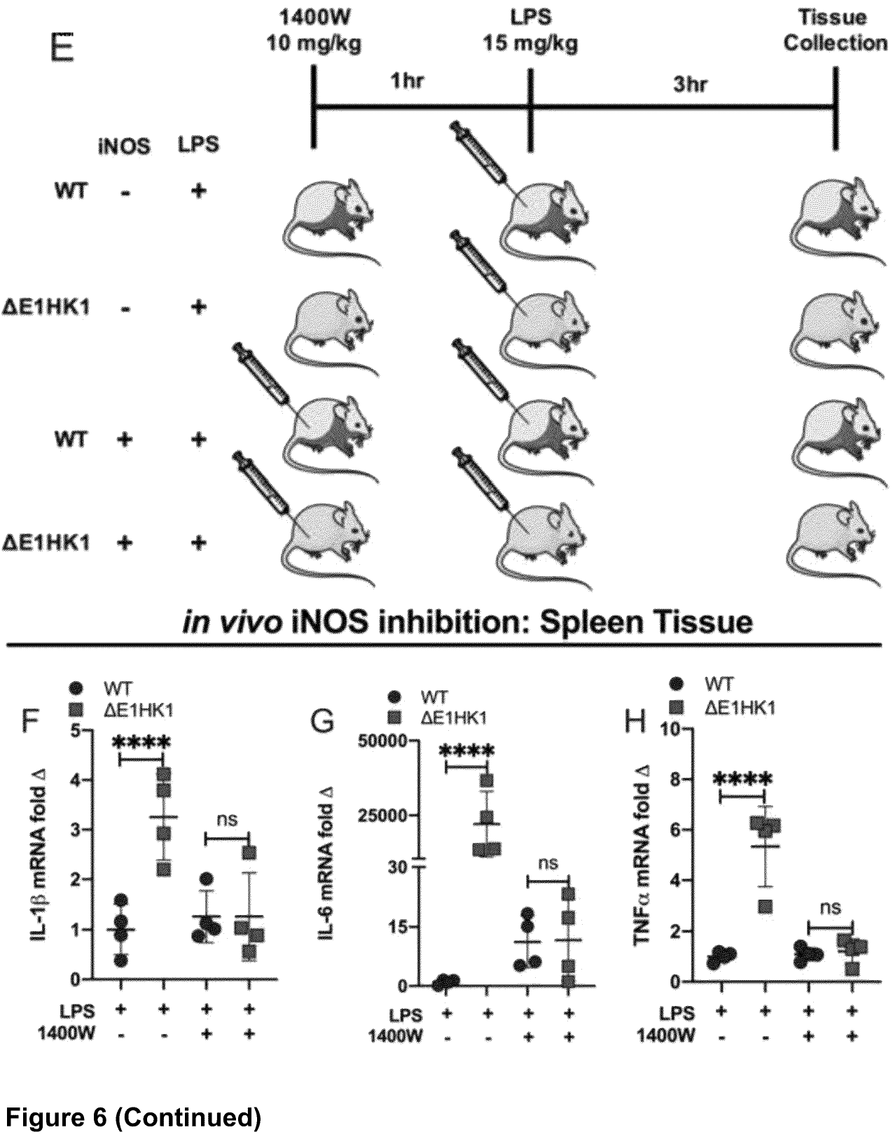

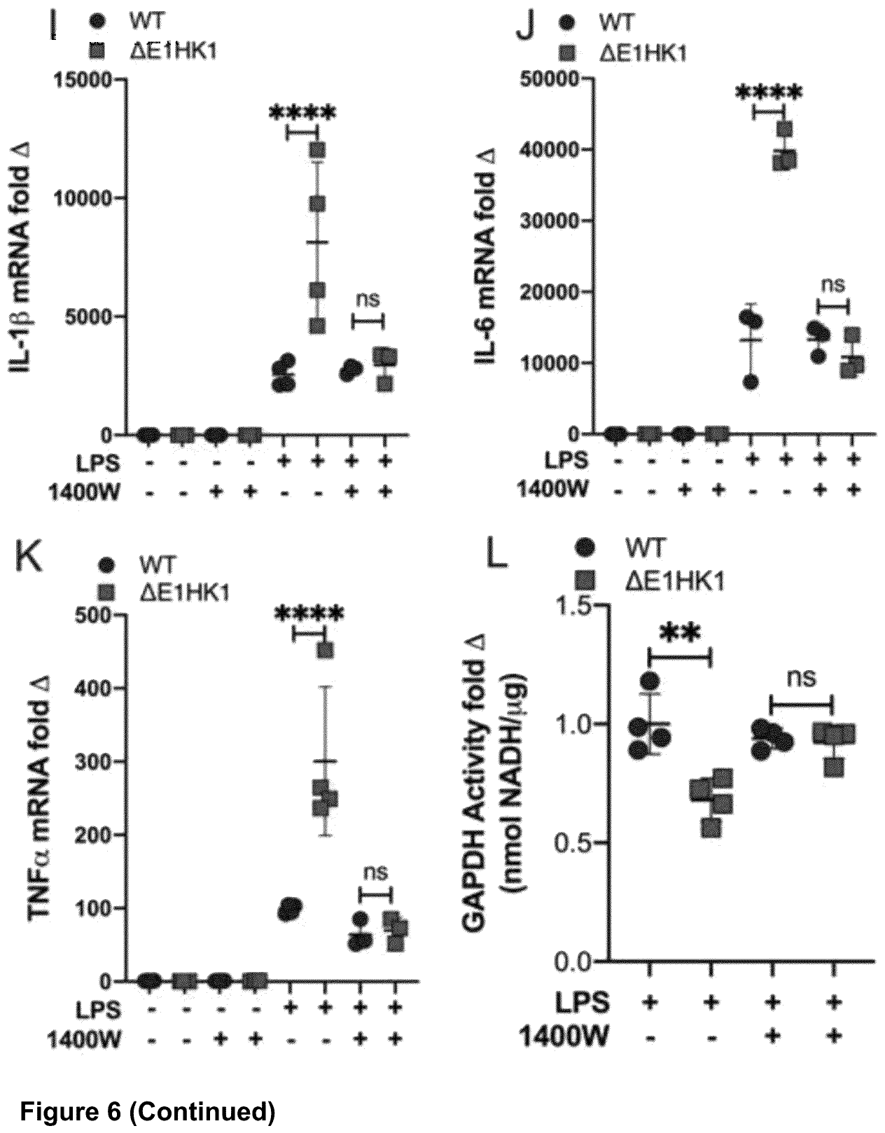

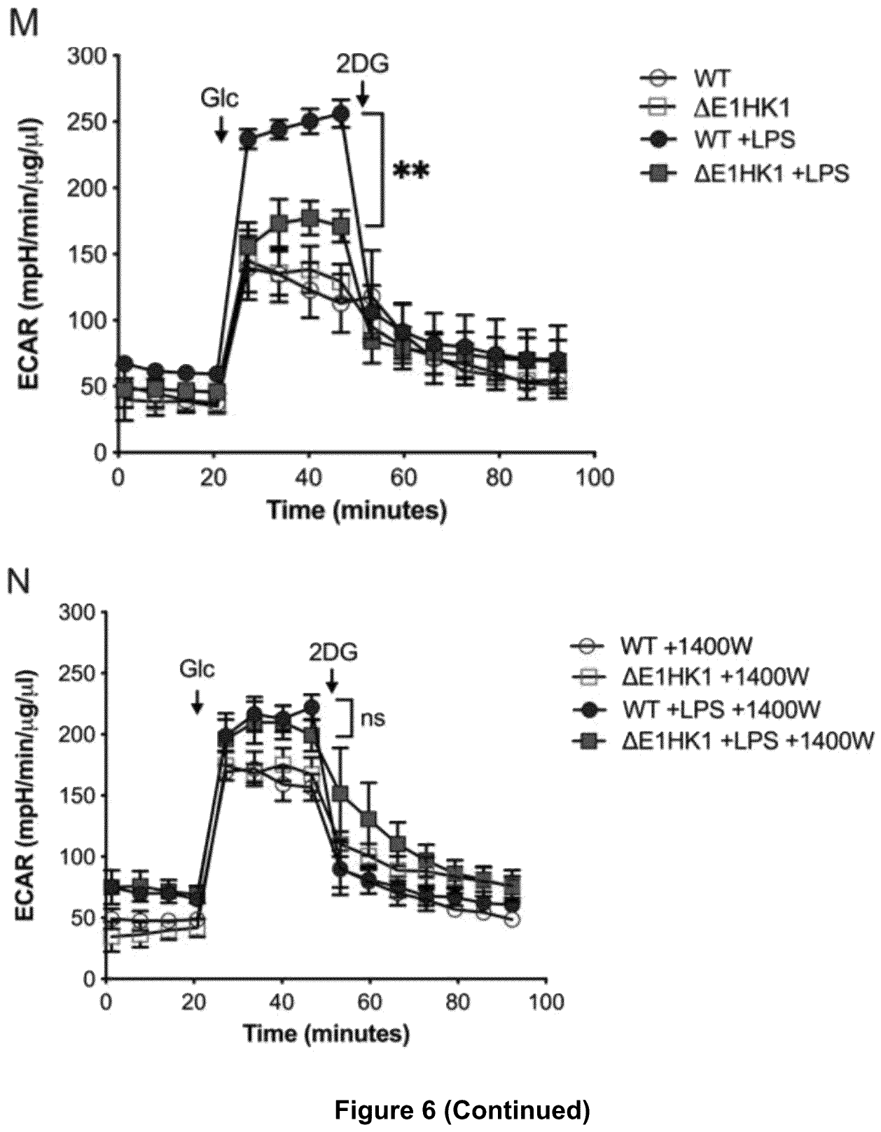

We then confirmed this finding in BMDMs from ΔE1HK1 mice and found S100A8 as a binding partner of cytosolic HK1 in these cells (FIGS. 6A and 6B). S100A8 and S100A9 form a protein dimer called calprotectin that can bind to iNOS in macrophages and mediate trans-S-nitrosylation of GAPDH (SNO-GAPDH) and inhibition of its glycolytic activity (Jia et al., 2014). We thus assessed GAPDH nitrosylation in TrHK1 overexpressing cells, and showed that cells with overexpression of TrHK1 display higher GAPDH nitrosylation levels (FIG. 13G). Importantly, BMDMs from ΔE1HK1 mice also displayed higher GAPDH nitrosylation than WT controls, which was reversible with 1400W treatment, a selective iNOS inhibitor (Thomsen et al., 1997) (FIGS. 6C and 6D). To assess whether the hyper-inflammation seen in ΔE1HK1 BMDMs is dependent on protein nitrosylation through iNOS, we administered 1400W to mice 1 hour prior to intraperitoneal injection of a sub-lethal dose of LPS for 3 hours and collected blood and splenic tissue from these mice for analysis (FIG. 6E). We observed a rescue of the hyper-inflammatory response seen in ΔE1HK1 mice as measured by splenic mRNA (FIG. 6F-H) and serum ELISA of inflammatory cytokines IL-1β, IL-6, and TNFα (FIG. 13H). Additionally, treatment of BMDMs isolated from ΔE1HK1 mice with 1400W caused reversal of the hyper-inflammatory response as measured by mRNA of IL-1β, IL-6, and TNFα (FIG. 6I-K). GAPDH activity of ΔE1HK1 BMDMs was also restored to WT levels after 1400W treatment (FIG. 6L). Next, we assessed whether iNOS inhibition could reverse the altered glycolytic effects seen in ΔE1HK1 BMDMs. We found that iNOS inhibition eliminated the differences in ECAR observed between ΔE1HK1 and WT BMDMs (FIGS. 6M and 6N). Together, these data indicate that cytosolic HK1 binds to the S100A8 and promotes iNOS mediated nitrosylation and inactivation of GAPDH.

Diabetes and Aging are Associated with HK1 Mitochondrial Dislocation and Increased Cytokine Production

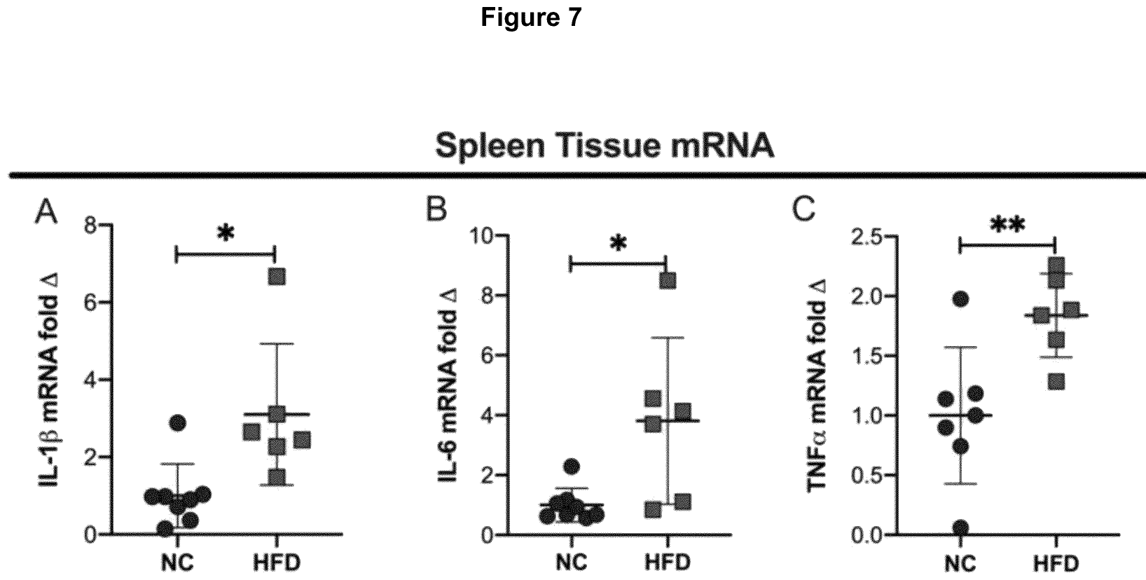

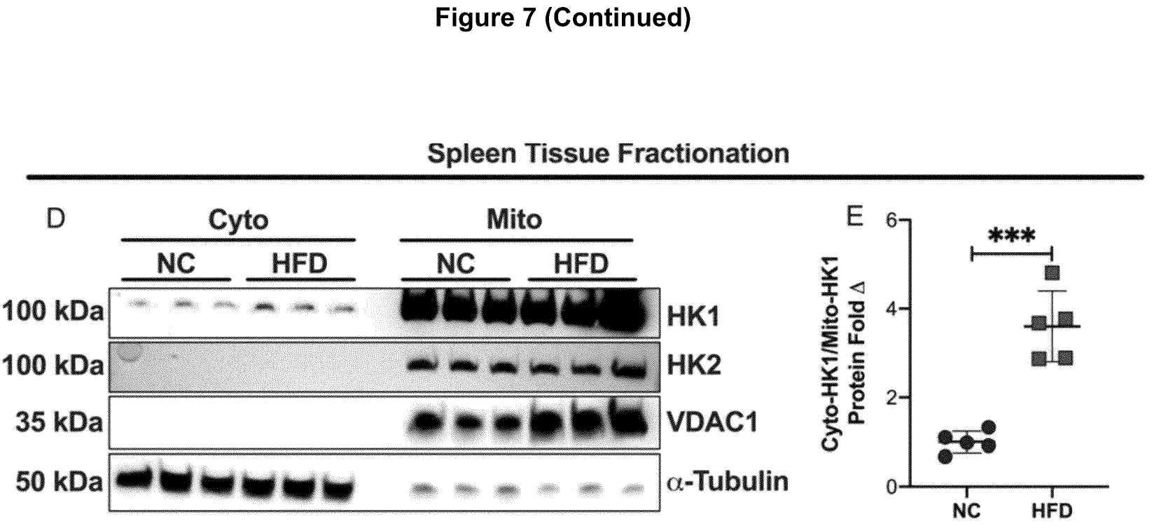

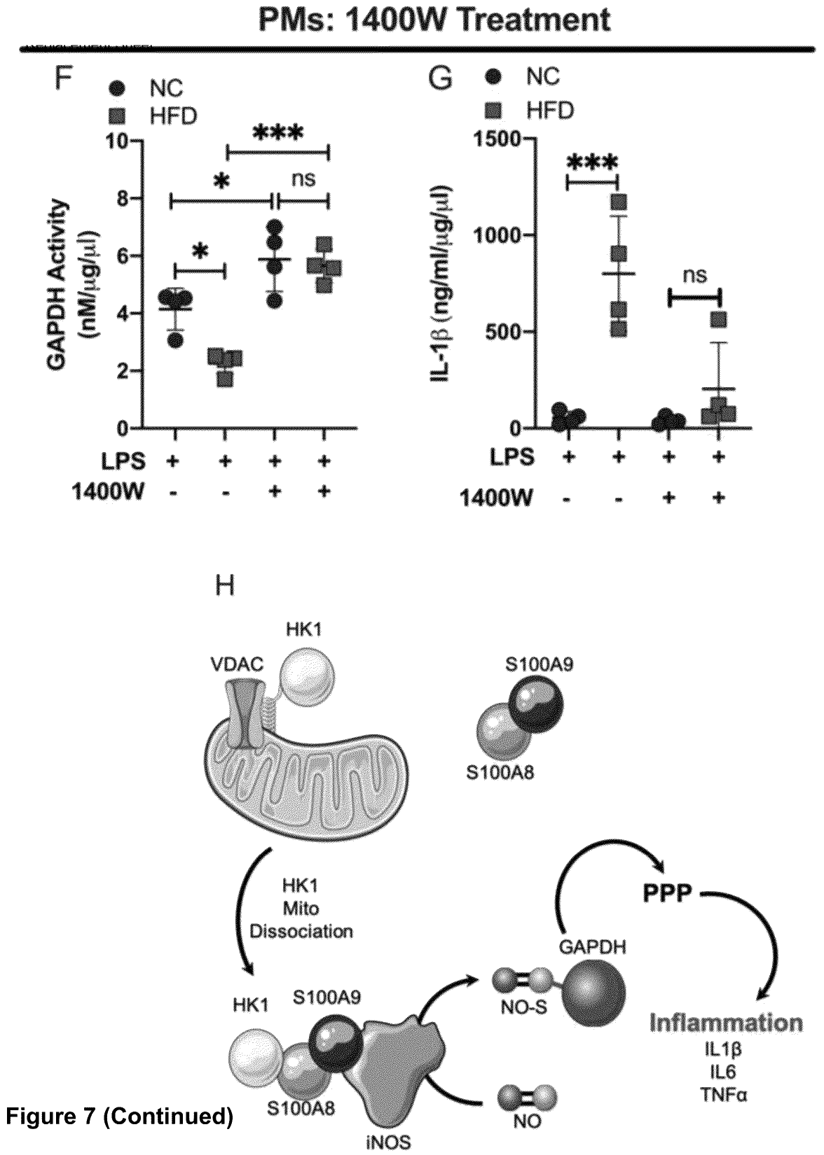

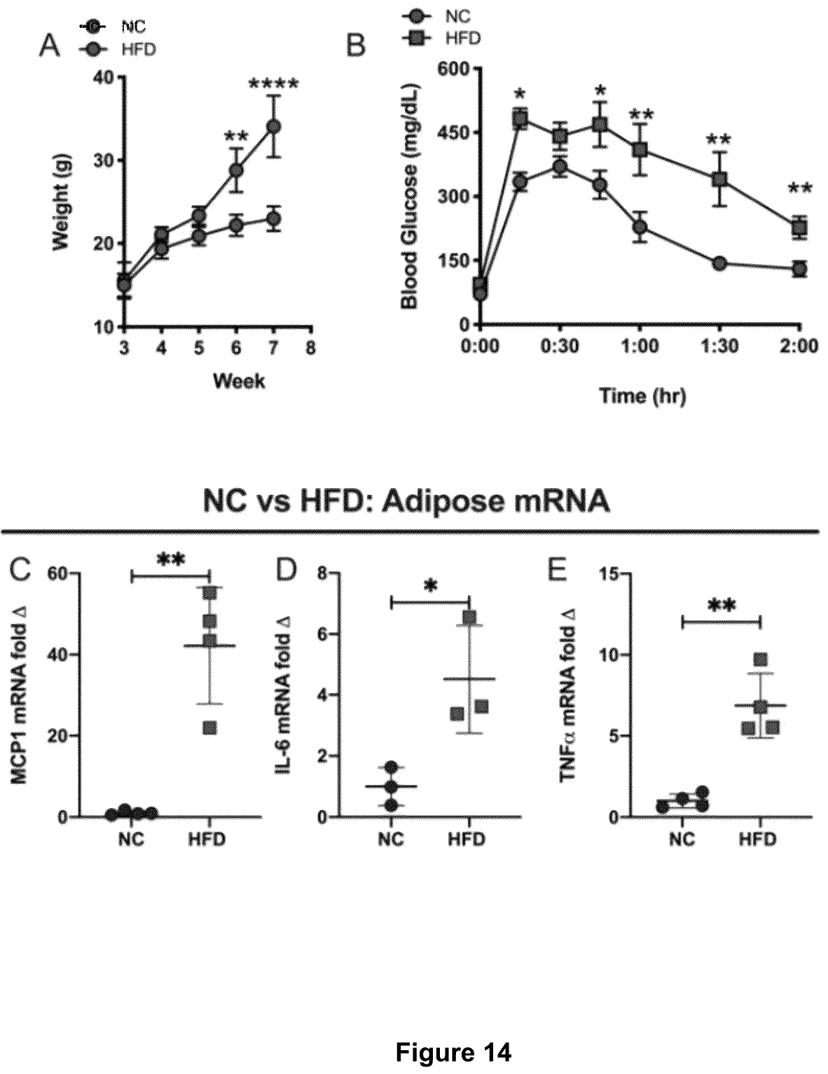

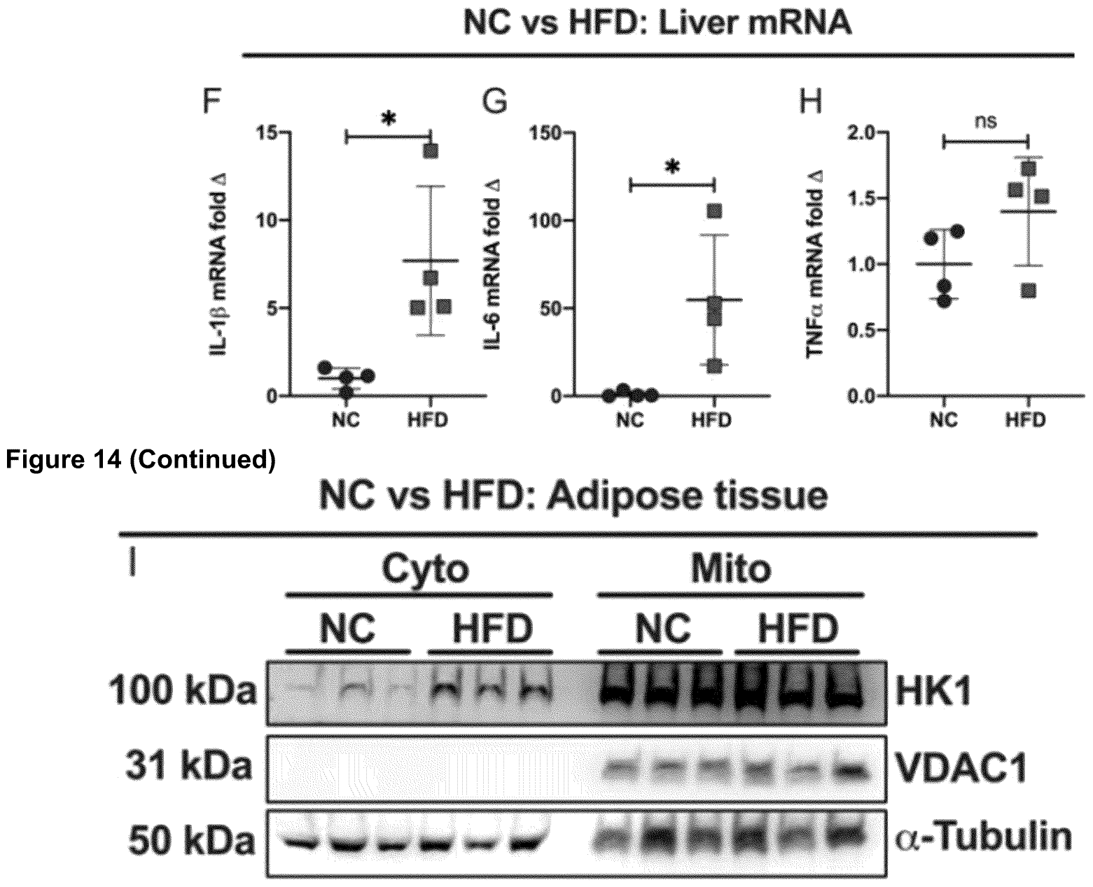

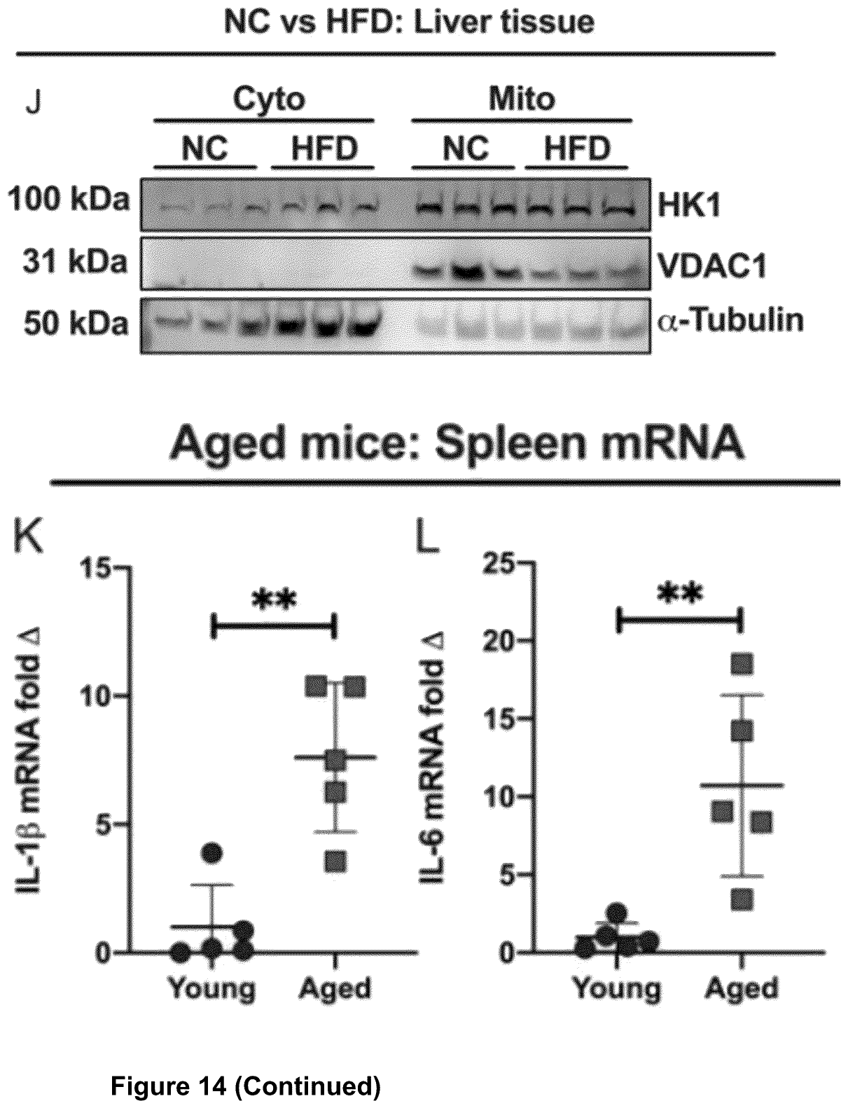

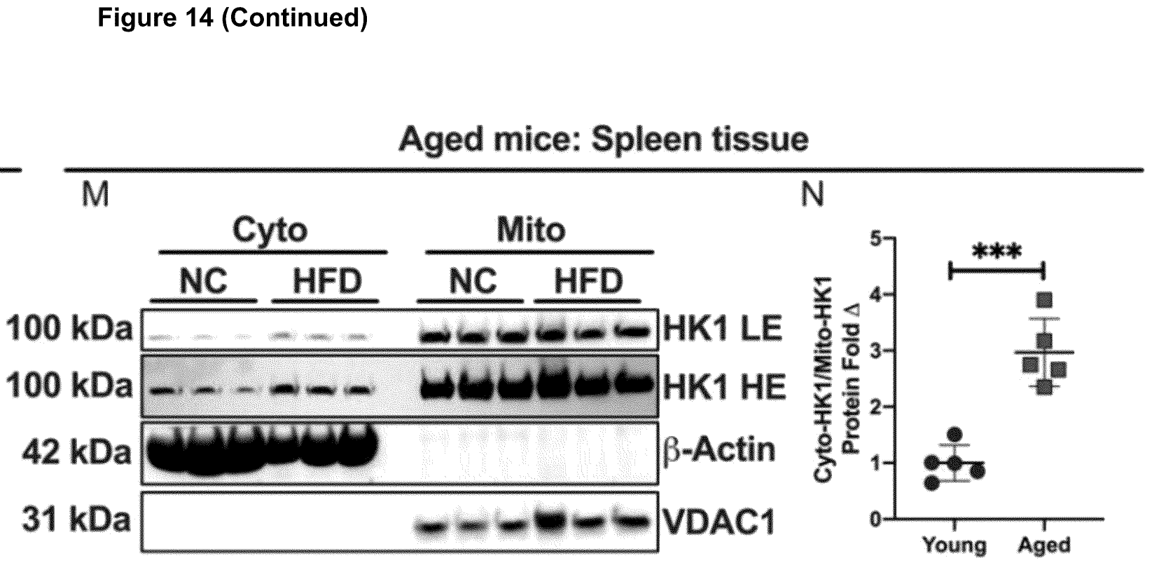

Our data thus far indicates that constitutive or acute HK1 dislocation causes an increase in cytokine production through inhibition of GAPDH, which alters glycolysis and subsequently increases PPP metabolism. We next studied whether subcellular localization of HK1 is altered in conditions of chronic low-grade inflammation, such as diabetes and aging. To generate diabetic mice, we fed 4-week-old mice a high fat diet (HFD) consisting of 60% fat, 20% protein, and 20% carbohydrates. Mice given HFD for 27 weeks displayed impaired glucose tolerance and increased body weight as compared to mice fed normal chow (NC) (FIGS. 14A and 14B). Splenic tissue from HFD mice displayed higher IL-1β, IL6, and TNFα mRNA levels than NC fed mice, which is consistent with low-grade inflammation (FIG. 7A-C). We observed a similar increase in inflammatory mRNA markers in adipose and liver tissues from HFD mice (FIGS. 14C-E and 14F-H). Additionally, spleens from HFD mice had an increased cytosolic to mitochondrial HK1 ratio than NC fed mice (FIGS. 7D and 7E). We observed a similar increase in cytosolic HK1 levels in adipose and liver tissue taken from HFD mice (FIGS. 141 and 14J). Elevated inflammation is also observed in aged mice, as assessed by measurement of IL-1β and IL-6 mRNA isolated from spleens of 85-week-old mice (FIGS. 14K and 14L). Aged mice also displayed increased cytosolic HK1 levels similar to HFD mice (FIGS. 14M and 14N). To assess whether this elevated inflammation was dependent on GAPDH activity, we isolated PMs from NC and HFD mice and found that the HFD group had lower GAPDH activity before and after LPS stimulation, which could be rescued with 1400W treatment (FIG. 7F). Furthermore, iNOS inhibition with 1400W reversed the elevation in IL-1β observed in the HFD PMs back to WT levels (FIG. 7G). Collectively, these data indicate that HK1 mitochondrial dissociation occurs in diseases of chronic-low grade inflammation, and restoration of GAPDH activity with iNOS inhibition can rescue this phenotype in PMs derived from diabetic mice.

### Discussion

Here, we show that HK1 mitochondrial dissociation produces a metabolic block at the level of GAPDH, which increases PPP metabolism. This feature of HK1 regulation is dependent on its localization between the cytosol and mitochondria and is independent of its enzymatic activity, since removal of HK1 MBD does not change its enzymatic function. We also demonstrate that the increased PPP metabolism has functional consequences, as dislocation of HK1 to the cytoplasm results in higher cytokine production and exaggerated inflammatory response to endotoxemia in vivo. Additionally, we show that the mechanism for the altered G6P metabolism by HK1 cellular distribution is through increased GAPDH S-nitrosylation and subsequent attenuation of its enzymatic activity. Our data suggest that cytosolic HK1 binds to S100A8/A9, leading to S-nitrosylation of GAPDH through iNOS. Furthermore, we find that inhibition of iNOS is sufficient to reverse the metabolic alterations and elevated cytokine production seen with mitochondrial dissociation of HK1. Therefore, HK1 subcellular localization is a critical modulator of glycolysis, and regulates the inflammatory response in macrophages (FIG. 7H).

The hydrophobic N-terminal domain of HK1 and HK2 encoded by exon 1 allows the proteins to bind to the mitochondria (John et al., 2011; Wilson, 2003). We previously showed that the mitochondrial binding of HK1 and HK2 are needed for their protective effects against cell death (Sun et al., 2008). Other reports have suggested that the binding of HK1 and HK2 to the mitochondrial allows preferential access of these enzymes to mitochondrial-produced ATP that is transported through VDAC, in addition to effective delivery of ADP that is produced by glucose phosphorylation to ANT for transport to mitochondrial matrix (BeltrandelRio and Wilson, 1992; Golshani-Hebroni and Bessman, 1997; Rosano et al., 1999). However, the Kₘ for ATP of these enzymes is significantly lower than cytosolic ATP (Zeng et al., 1996; Rosano et al., 1999), making the validity of this hypothesis dubious. Thus, the physiological significance of HK1 binding to the mitochondria is not clear. G6P can shuttle through different pathways (Adeva-Andany et al., 2016), and despite extensive work on glucose metabolism, there is a paucity in our understanding of how the metabolic fate of G6P is determined. Here, we identify a mechanism by which the localization of HK1, independent of its enzymatic activity, regulates the metabolic fate of its enzymatic product, G6P, and increases its shunting into the PPP. These data highlight how subcellular localization of a metabolic enzyme determines the fate of its product.

We also showed that the mechanism by which cytosolic HK1 increases PPP in macrophages is through GAPDH nitrosylation and enzymatic inhibition. A role for GAPDH nitrosylation in inflammation has been previously reported. Nitrosylation of GAPDH has been previously shown to increase inflammation in macrophages (Jia et al., 2014; Padgett and Whorton, 1995), and GAPDH inhibitors cause an increase in IL-1β secretion by macrophages (Sanman et al., 2016). Additionally, a role for increased PPP metabolites in inflammation has been suggested before (Baardman et al., 2018; Haschemi et al., 2012). Our results provide a biological context and mechanism for GAPDH regulation and links these steps to HK1 mitochondrial binding. We showed that HK1 dislocation from the mitochondria leads to its association with S100A8/A9, nitrosylation of GAPDH and its subsequent inhibition. GAPDH is a critical step in glycolysis in that the substrates above this step can enter the PPP, and thus its inhibition would block glycolysis, while allowing shuttling of upstream intermediates into PPP. We suspect that this system was developed in mammalian cells to serve as a means of regulating the metabolic fate of G6P into ancillary pathways off glycolysis, such as the PPP. Since the metabolic block at GAPDH increases metabolite levels above this point, it would be interesting to investigate how other metabolic branch points of G6P (i.e., glycogenesis and hexosamine pathway) are affected in other contexts.

In light of our findings, it is now important to determine both physiological and pharmacological factors that modulate HK1 binding to the mitochondria and its release into the cytoplasm. HK1 dislocation from the mitochondria has been reported to occur in response to a number of pathways and processes, including senescence, ROS, CLT, inhibition of AKT, and activation of GSK (Gardiner et al., 2007; Saraiva et al., 2010; John et al., 2011; Fouquerel et al., 2014; Sen et al., 2015; Hauser et al., 2017; Bantug et al., 2018; Mogilenko et al., 2019). These findings indicate that there is shuttling of HK1 from the mitochondria to the cytoplasm under physiological and pathological conditions. In addition, a synthetic peptide with sequence homology to the N-terminus of HK1 can be used to displace the proteins from the mitochondria (Magri et al., 2017). Thus, this peptide can potentially be used in clinical settings to induce HK1 displacement from mitochondria and increase the inflammatory response in conditions of impaired inflammation, such as immunosuppressive disorders. We also showed that in the setting of certain conditions associated with low-grade inflammation, such as aging and diabetes, there is endogenous HK1 dislocation from the mitochondria, increased production of inflammatory markers, and diminished GAPDH activity with LPS stimulation. Additionally, treatment with an iNOS inhibitor showed a cell-autonomous reversal of the elevated cytokine production observed in DIO PMs along with restoration of GAPDH activity. These results indicate that HK1 mitochondrial binding may mediate the inflammation underlying the pathogenesis of diabetes and aging. Thus, it would be important to devise strategies to maintain HK1 on the mitochondria to reduce or ablate the inflammation (either low-grade or fulminant) that contributes to the negative sequelae of these and other inflammation-associated disorders.

In summary, we have identified a novel mechanism by which the product of the HK enzymatic reaction, G6P, is directed towards the PPP at the expense of a reduction in lower glycolytic intermediates. This metabolic effect is regulated through HK1 mitochondrial interaction and that the mitochondrial dissociation of this enzyme causes an increase in PPP through inhibition of GAPDH. We provide data suggesting this mechanism is mediated through HK1 interaction with S100A8/A9, which increased iNOS activity and subsequent GAPDH nitrosylation and inhibition. These results provide a new mechanism by which the subcellular localization of a glycolytic enzyme regulates its own downstream metabolism to produce an effect on inflammatory cytokine production.

### Materials and Methods

TABLE 1
Key resources table.

| | | |
|---|---|---|
| REAGENT or RESOURCE | SOURCE | IDENTIFIER |

Antibodies

| | | |
|---|---|---|
| S100A8 Rabbit Polyclonal Antibody | Proteintech | 15792-1-AP |
| ATP Synthase beta Monoclonal-Alexa Fluor 555 | Thermo Fisher Scientific | MA1930A555 |
| GFP-tag Mouse Monoclonal Antibody | Proteintech | 66002-1-Ig |
| VDAC1/Porin Rabbit Polyclonal Antibody | Proteintech | 55259-1-AP |
| Hexokinase I (C35C4) Rabbit mAb | Cell Signaling | 2024S |
| | Technologies | |
| Hexokinase II (C64G5) Rabbit mAb | Cell Signaling | 2867 |
| | Technologies | |
| IL-1β (3A6) Mouse mAb | Cell Signaling | 12242 |
| | Technologies | |
| GAPDH (14C10) Rabbit mAb | Cell Signaling | 2118S |
| | Technologies | |
| GFP-Trap Agarose | Chromotek | gta-20 |
| Binding Control Agarose Beads | Chromotek | bab-20 |
| Alpha-Tubulin Antibody | Proteintech | 66031-1-Ig |
| TMT Monoclonal Antibody (25D5) | Thermo Fisher Scientific | 90075 |
| Beta-Actin Antibody | Proteintech | 60008-1-Ig |
| FLAG M2 mouse | Sigma-Aldrich | F1804 |
| Goat anti-Rabbit IgG (H + L), Superclonal ™ | Thermo Fisher Scientific | A27034 |
| Secondary Antibody, Alexa Fluor 488 | | |
| Donkey Anti-Mouse IgG (H + L) Antibody | Jackson ImmunoResearch | 715-035-150 |
| Horseradish Peroxidase (HRP) | | |
| Donkey Anti-Rabbit IgG (H + L) Antibody | Jackson ImmunoResearch | 711-035-152 |
| Horseradish Peroxidase (HRP) | | |

Bacterial and Virus Strains

| | | |
|---|---|---|
| pHIV-Puro-EGFP (lentivirus) | Disclosed herein | N/A |
| pHIV-Puro-FLHK1-EGFP (lentivirus) | Disclosed herein | N/A |
| pHIV-Puro-TrHK1-EGFP (lentivirus) | Disclosed herein | N/A |

Chemicals, Peptides, and Recombinant Proteins

| | | |
|---|---|---|
| Lactate Oxidase from Aerococcus Viridans | Sigma-Aldrich | L9795 |
| β-Nicotinamide adenine dinucleotide phosphate | Sigma-Aldrich | N8035-15VL |
| sodium salt | | |
| Pierce 16% formaldehyde | Thermo Fisher Scientific | 28906 |
| Clotrimazole | Sigma-Aldrich | C6019 |
| 2-Deoxy-D-glucose | Sigma-Aldrich | D8375 |
| IFN gamma mouse recombinant protein | PeproTech | 315-05-100ug |
| RNA stat 60 | Tel Test Inc | CS-502 |
| Novex ™ 16% Tricine Protein Gels, 1.0 mm, 12- | Thermo Fisher Scientific | EC66952BOX |
| well | | |
| Accutase | Sigma-Aldrich | A6964 |
| Red Blood Cell (RBC) Lysis Buffer | Abcam | Ab204733 |
| Macrophage-Colony Stimulating Factor (mCSF)- | GenScript | Z02930 |
| mouse | | |
| Methyl pyruvate | Sigma-Aldrich | 371173 |
| CGP 3466B maleate | Tocris | 2966 |
| Heptelidic Acid (Koningic Acid) | Cayman Chemical | 14079 |
| | Company | |
| 1400W dihydrochloride | Tocris | 1415 |
| 6-Aminonicotinamide | Sigma-Aldrich | A68203 |
| Oxythiamine chloride hydrochloride | Sigma-Aldrich | 04000 |
| DMEM with L-Glutamine | Corning | MT-10-017- |
| | | CV |
| RPMI 1640 with L-Glutamine | Sigma-Aldrich | R1383 |
| Digitonin | Sigma-Aldrich | D141 |
| ProLong Gold Antifade Mountant with DAPI | Life Technologies | P36931 |
| MitoTracker Deep Red FM | Thermo Fisher Scientific | M22426 |
| Tetramethylrhodamine, Ethyl Ester, Perchlorate | Thermo Fisher Scientific | T669 |
| (TMRE) | | |
| Ultrapure O5:B55 LPS | Invivogen | tlrl-pb5lps |
| Crude O5:B55 LPS | Sigma | L2880 |
| D-Glucose (U-13C6) | Cambridge Isotope | CLM-1396-1 |
| | Laboratories | |
| 2-NBDG | Cayman Chemical | 11046 |
| | Company | |
| Glucose-6-Phosphate Dehydrogenase (G6P-DH) | Sigma | 10165875001 |
| from Leuconostoc mesenteroides | | |
| ATP | Sigma-Aldrich | A2383 |
| Tween-20 | DOT Scientific Inc | DSP20370-0.5 |
| TRUE Metrix Blood Glucose Test Strips 5 | Trividia Health Inc. | N/A |

Critical Commercial Assays

| | | |
|---|---|---|
| NADP/NADPH-Glo kit | Promega | G9081 |
| NAD/NADH-Glo kit | Promega | G9071 |
| Duoset ELISA IL-1beta-mouse kit | R&D | DY401-05 |
| Duoset ELISA TNFalpha-mouse kit | R&D | DY410-05 |
| Duoset ELISA IL-6-mouse kit | R&D | DY406-05 |
| Anti-HK1 Magnetic Beads IP kit | Sino Biological | MB101347- |
| | | T38 |
| Infusion HD kit | Clontech | 638910 |
| Glyceraldehyde 3 Phosphate Dehydrogenase | Abcam | ab204732 |
| Activity Assay kit | | |
| Thermo Scientific Pierce S-Nitrosylation | Fisher Scientific | PI90105 |
| Western Blot kit | | |
| Pierce BCA Protein Assay kit | Fisher Scientific | PI23225 |
| Mitochondria Isolation Kit for Cultured Cells | Fisher Scientific | PI-89874 |

Experimental Models: Cell Lines

| | | |
|---|---|---|
| Human: HEK293T | ATCC | Cat# CRL- |
| | | 3216 |
| Human: HepG2 | ATCC | Cat# HB-806 |
| Mouse: RAW264.7 | Prof. Jason Albert | N/A |
| | Wertheim (Northwestern | |
| | University) | |
| Mouse: primary bone marrow-derived | Disclosed herein | N/A |
| macrophages | | |
| Mouse: primary peritoneal macrophages | Disclosed herein | N/A |

Experimental Models: Organisms/Strains

| | | |
|---|---|---|
| ΔE1HK1-C57BL/6J mice | Disclosed herein | N/A |
| WT C57BL/6J mice | Disclosed herein | N/A |

Recombinant DNA

| | | |
|---|---|---|
| FLHK1-pGFPN3 | Inventors | N/A |
| TrHK1-pGFPN3 | Inventors | N/A |

Software and Algorithms

| | | |
|---|---|---|
| MetaboAnalyst 4.0 | Xia, Wishart, & Chong, | |
| | 2019 | |
| Scaffold | Proteome Software | |
| Prism 8.0 | Graphpad | |
| PANTHER classification system | Gene ontology Unifying | |
| | Biology | |
| Fiji/ImageJ | NIH | RRID: |
| | | SCR_002285 |

Other

| | | |
|---|---|---|
| High Fat Diet (HFD; 60% kcal fat) | Research diets | Cat# D12492 |

### Experimental Model and Subject Detail

### Cell Lines and Culture

RAW264.7 cells were a gift from Jason A. Wertheim MD, PhD. HepG2 and HEK293T cells were obtained from ATCC. RAW264.7, HEK293T, and HepG2 cells were cultured in Dulbecco's Modified Eagle's Medium (DMEM) (Corning) supplemented with 10% FBS (Atlanta Biologicals), 2 mM glutamine (HyClone), and 1 mM sodium pyruvate (Corning). RAW264.7 cells were treated with 300 ng/ml LPS for 4-6 hrs with or without CLT (50 μM).

### Mouse Line

HK1^{ΔE1HK1/ΔE1HK1} (ΔE1HK1) and HK1^{WT/WT} wild type (WT) control mice were generated by the Northwestern Mutagenesis and Transgenic core. Mouse genetic background is C57BL/6J. Mice were maintained in the barrier facility at Northwestern University under specific pathogen-free conditions in accordance with Federal and University guidelines and protocols approved by Institutional Animal Care and Use Committee (IACUC) with 12 hr light and 12 hr dark cycle, and received normal chow. Male and female mice were used at 8-12 weeks of age. HFD and NC diet mice were maintained on respective diets for 27 weeks. All animal studies were approved by the IACUC at Northwestern University and were performed in accordance with guidelines from the National Institutes of Health.

### Peritoneal Macrophages Isolation and Cell Culture

Peritoneal macrophages were isolated from 8-10-week-old ΔE1HK1 and littermate control WT mice by peritoneal lavage as described previously (Ray and Dittel, 2010). Briefly, mice were euthanized and the peritoneal cavity was injected with PBS supplemented with 5% FBS using a 27g needle. The abdominal wall was gently agitated to dislodge peritoneal cells. Using a fresh 25g needle, the peritoneal lavage (PBS and peritoneal cells) was aspirated and collected. Cells were then centrifuged at 500 g for 10 min and re-suspended in 1 mL of complete RPMI (Corning) supplemented with 10% FBS (Atlanta Biologicals), 2 mM glutamine (HyClone), 1 mM HEPES (Corning) and 1 mM pyruvate (Corning). Stimulation with LPS (Invivogen) was performed the next day. 300 ng/ml of LPS was given to the cells for 4-6 hrs with or without the following drugs; OT (50 μM), 6-AN (1 mM), 1400W (50 μM), or CGP3466 (50 nM).

### BMDM Isolation and Cell Culture

BMDMs were isolated from ΔE1HK1 and WT mice as previously reported (Rodriguez et al., 2019). Briefly, bone marrow was isolated from the tibia and femur of 8-10-week-old mice by puncturing one end of the femur and tibia with a 27g needle and placing it in a 0.5 ml tube with a hole punched in the bottom. The 0.5 ml tube containing the bone was then placed in a 1.75 ml tube and centrifuged at 5,000×g for 3.5 minutes. Bone marrow was then collected into the 1.75 ml tube and resuspend in 1 ml of RBC lysis buffer (abcam) for 1 minute and then transferred to a 50 ml tube with 4 ml RBC lysis buffer and incubated for 4 minutes. Next, 35 ml of PBS was added and centrifuged at 300×g for 5 min and supernatant was then decanted and cell pellet was re-suspended in complete RPMI with 20 ng/ml mCSF (Preprotech). Cells were counted using a hemocytometer and plated in 10 cm tissue culture plates at a density of 3-million cells/plate. Cells were cultured with 20 ng/mL mCSF to induce differentiation into BMDMs. Media was changed every 2 days and BMDMs were harvested by scraping on day 6 and plated in 12 well plates at a density of 1-million cells/well for mRNA extraction experiments. BMDMs were stimulated in the same manner as peritoneal macrophages (LPS dose for BMDMs was 200 ng/ml).

### Method Details

### Generation of HK1 Lentivirus Gene Overexpression Constructs

Lentivirus C-terminal GFP-tag fusion over-expression constructs for EV, FLHK1, and TrHK1 were cloned into the pHIV-Puro vector (generated in our lab) using InFusion-HD cloning method. Lentiviral particles were produced in HEK293T cells co-transfected with pSPAX2 and pMD2.G packaging vectors using standard protocols. To generate the full-length and truncated HK1-GFP fusion plasmids, In-Fusion cloning PCR primers were designed to the first or second exon of the 2754-bp fragment from the EGFP-N3 plasmid containing FLHK1 or TrHK1. To perform the In-Fusion reaction, the PCR primers also contained overhang regions containing XhoI and BamHI-HF restriction enzyme sites and a 15-bp homology portion to the pHIV-Puro vector to facilitate recombination.

### RNA Isolation, Reverse Transcription and Quantitative RT-PCR

RNA was isolated from cells or tissues using RNA-STAT60 (Teltest) followed by chloroform extraction and precipitation. Reverse transcription was carried out using qScript cDNA Synthesis Kit (Quanta Bio). The resulting cDNA was amplified quantitatively using PerfeCTa SYBR Green Mix (Quanta Bio) on a 7500 Fast Real-time PCR System (Applied Biosystems). The relative gene expression was determined using differences in Ct values between gene of interest and house-keeping control genes. Complete list of primers can be found in Key Reagents Table (Table 1).

### Western Blots

Cells and tissue were lysed in radio-immunoprecipitation assay (RIPA) buffer supplemented with 1× protease inhibitor (G-Bioscience). Protein concentration in samples was determined using the BCA Protein Quantification Kit (Pierce). Equal amounts of protein were loaded on a tris-glycine polyacrylamide gel (Life Technologies) and transferred to nitrocellulose membrane. After blocking with tris-buffered saline containing 0.05% Tween 20 (DOT Scientific Inc) and 5% BSA, the membrane was incubated with primary antibody against indicated proteins. A complete list of antibodies is included in Key Resources Table (Table 1). For low molecular weight protein S100A8 western blots, 16% tricine protein gels (ThermoFisher) were used and western blots run as previously described (Schägger, 2006). Western blot densitometry was performed using Fiji/ImageJ Gel analyzer macro (Schindelin et al., 2012).

### Confocal Imaging

Glass-bottom confocal dishes (35 mm; VWR) were coated with 60 nM fibronectin (Sigma-Aldrich), diluted in 0.1% gelatin overnight. Before plating cells, coated confocal dishes were washed twice with PBS. BMDMs (100,000 cells/well), PMs (100,000 cells/well), HepG2 (50,000 cells/well), or RAW264.7 (50,000 cells/well) cells were plated in their respective growth media (complete DMEM or RPMI) and allowed to settle overnight before immunofluorescence (IF) staining. For fixed cell immunofluorescence (IF), cells were plated in 6-well plates (Corning) containing 15 mm glass coverslips (Fisher-Scientific) coated in gelatin and fibronectin. Prior to fixation, growth media was removed and cells were washed 2× with PBS. Cells were fixed with 1 ml of ice-cold 4% formaldehyde for 10 min at room temperature (4% formaldehyde prepared from 16% stock and diluted in PBS). After fixing the cells, plates were washed with PBS 2× and permeabilized with 0.3% triton-X-100 in PBS for 10 min at room temperature. Then, cells were blocked with 10% FBS/PBS for 1 hour at room temperature and subsequently treated with primary antibody diluted in 10% FBS/PBS solution over-night. The next day, secondary antibody was diluted in 10% FBS/PBS at 1:1000 and incubated for 2 hrs at room temperature. Cells were then washed 3× with PBS and mounted on glass slides (Fisher-Scientific) using ProLong Gold Antifade Mountant with DAPI and imaged using confocal microscope. HK1 antibody diluted at 1:100, ATP Synthase-beta (ATP5B) Monoclonal-Alexa Fluor 555 antibody diluted at (1:200), and MitoTracker Deep Red FM (1 μM). All images were acquired on a Zeiss LSM 510 Meta confocal microscope. Images were quantified using ImageJ.

### TMRE Colocalization

HepG2 cells with EV, FLHK1, or TrHK1 overexpression were washed with PBS and changed to confocal buffer (25 mM D-glucose, 1.8 mM CaCl2, 2.5 mM KCl, 140 mM NaCl, 2 mM sodium pyruvate, 2 mM glutamine, 20 mM HEPES, pH 7.5, 1 mM MgCl2). Cells were stained with 5 nM TMRE (for mitochondrial stain) for 20 minutes. Cells were then washed with PBS, and fresh confocal buffer was added. TMRE (red channel) and GFP (green channel) signals were analyzed for colocalization using ImageJ Coloc-2 function to determine Pearson correlation coefficient between green and red channel for confocal images.

¹³C₆-Glucose Tracing and Steady-State Metabolomics

Cultured BMDMs or HepG2 cells were treated with ¹³C₆-glucose for 4 hrs±LPS (200 ng/ml) and mass-spectrometry and metabolite identification was performed on 80% methanol & 20% ultrapure water extracted metabolites. Whole brain tissues were harvested from mice and immediately flash frozen in liquid nitrogen until harvested for metabolites using 80% methanol & 20% ultrapure water extraction protocol. Metabolomics services were performed by the Metabolomics Core Facility at Robert H. Lurie Comprehensive Cancer Center of Northwestern University. Samples were analyzed by High-Performance Liquid Chromatography and High-Resolution Mass Spectrometry and Tandem Mass Spectrometry (HPLC-MS/MS). Specifically, system consisted of a Thermo Q-Exactive in line with an electrospray source and an Ultimate3000 (Thermo) series HPLC consisting of a binary pump, degasser, and auto-sampler outfitted with an Xbridge Amide column (Waters; dimensions of 4.6 mm×100 mm and a 3.5 μm particle size). The mobile phase A contained 95% (vol/vol) water, 5% (vol/vol) acetonitrile, 20 mM ammonium hydroxide, 20 mM ammonium acetate, pH=9.0; B was 100% Acetonitrile. The gradient was as following: 0 min, 15% A; 2.5 min, 30% A; 7 min, 43% A; 16 min, 62% A; 16.1-18 min, 75% A; 18-25 min, 15% A with a flow rate of 400 μL/min. The capillary of the ESI source was set to 275° C., with sheath gas at 45 arbitrary units, auxiliary gas at 5 arbitrary units and the spray voltage at 4.0 kV. In positive/negative polarity switching mode, an m/z scan range from 70 to 850 was chosen and MS1 data was collected at a resolution of 70,000. The automatic gain control (AGC) target was set at 1×106 and the maximum injection time was 200 ms. The top 5 precursor ions were subsequently fragmented, in a data-dependent manner, using the higher energy collisional dissociation (HCD) cell set to 30% normalized collision energy in MS2 at a resolution power of 17,500. The sample volumes of 10 μl were injected. Data acquisition and analysis were carried out by Xcalibur 4.0 software and Tracefinder 2.1 software, respectively (both from Thermo Fisher Scientific).

### Glucose Uptake Assay

2-NBDG glucose uptake assay of LPS-activated BMDMs from WT and ΔE1HK1 mice was performed based on previous reports (Alonso-Castro and Salazar-Olivo, 2008). Briefly, cells were cultured overnight in 96 well plates and treated the next day with 300 μM 2NBDG for 1 hr and then quickly washed 3× with PBS. Cells were then re-suspended in RIPA buffer and imaged in a fluorescent plate reader.

### Extracellular IL-1β Western Blot

BMDMs were stimulated with O5:B55 LPS (Invivogen) for 6 hours with addition of 2 mM ATP (Sigma) for 30 minutes to activate the cleavage of pro-IL-1β (35 kDa) to cleaved-IL-1β (17 kDa). Cell supernatant was collected and analyzed by ELISA according to manufacturer's instructions for IL-1β (DY401), IL-6 (DY406), and TNFα (DY410).

### S-Nitrosylation Western Blot of GAPDH Immunoprecipitation

GAPDH immunoprecipitation of HepG2 and BMDM cells was performed and nitrosylated cysteines were replaced with covalent binding of TMT using Pierce S-Nitrosylation western blot kit according to manufactures protocol. Briefly, free cysteines were blocked with MMTS reagent, lysates were treated with iodoTMT/ascorbate to induce replacement of unstable S-NO with stable S-TMT moiety. Western blot was then run on the TMT-replaced lysates and probed with GAPDH (dilution 1:8,000) and anti-TMT (dilution 1:1000).

### Seahorse Assay

The day before the assay, the Seahorse cartridge was placed in the XF calibrant and incubated overnight at 37° C. On the day of the assay, cells were seeded into the Seahorse 96-well plate at 15,000 cells/80 μl per well for HepG2 cells or 100,000 cells/80 μl per well for BMDMs. The plates were incubated at RT for 1 hour in glucose free complete DMEM or RPMI without bicarbonate or phenol-red to allow even distribution of cells across the well floor. Before placing the sample plates in the Seahorse XF96 Analyzer, medium volume was adjusted to 175 μl in each well. 11 mM or 25 mM glucose for HepG2 cells or BMDMs respectively, Oligomycin at 2 μM, CCCP at 10 μM, and 2DG at 2 μM each, diluted in DMEM, were injected sequentially into each well including control wells, containing only medium, following the standard Seahorse protocol. For acute injection of LPS, the first port of the drug cartridge was replaced with LPS at 200 ng/ml.

### Endotoxin-Induced Model of Sepsis

LPS induced sepsis model in mice was approved by Northwestern University Institutional Animal Care and Use Committee. For short term LPS induced cytokine quantification, C57/B16 mice (aged 10-12 weeks) were treated i.p. with or without 1400W (10 mg/kg) for 2 hrs prior to i.p. treatment with ultrapure 05:B55 LPS from Invivogen (15 mg/kg) i.p. for 4 hours. Whole blood samples were harvested via cardiac puncture after mice were euthanized. Cytokine production in serum from whole blood was measured using the Mouse IL-1β (DY401), IL-6 (DY406), and TNFα (DY410) ELISAs from R&D. For survival studies, Crude 05:B55 LPS (Sigma) was administrated i.p. at a sub-lethal dose of 15 mg/kg and mice were monitored over 72 hrs, every 2-4 hrs for survival and signs of deterioration to determine humane endpoints (Shrum et al., 2014).

### Commercial Assay Kits

The following kits were used according to the manufacturer's instructions: Promega NADP/NADPH quantification kit (G9081) and NAD/NADPH quantification kit (G9071); Glyceraldehyde-3-Phosphate Dehydrogenase Activity Assay kit (abcam); ELISAs for IL-1β (DY401), IL-6 (DY410), and TNFα (DY406).

CBC and WBC from Whole Blood

A HEMEVET blood cell analyzer was used on whole blood from mice using mouse standard blood as a control for comparing readouts.

### Co-Immunoprecipitation-Western Blot

Anti-HK1 magnetic beads IP kit (Sino Biological) was used to IP HK1 from BMDMs. BMDMs were plated in 15 cm culture dishes and lysed using NP40 cell lysis buffer (Sino Biological-provided in kit) and sonicated for 5 pulses for 1 sec each. Lysate was centrifuged at 8,000×g and supernatant was collected in a fresh tube. 50 μL of HK1-magnetic beads was added to afresh 1.7 mL tube and washed with 150 μL 1×TBS (10×TBS: 60.6 g Tris, 87.6 g NaCl, 1M HCl, 7.5 pH) with 0.5% Tween-20 (DOT Scientific Inc) and beads were precipitated with magnetic separator (Sino Biological-provided in kit). 1,000 ug of lysate from ΔE1HK1 and WT BMDMs was added to the precipitated magnetic HK1 beads and incubated overnight at 4° in rotator. Next day, beads were magnetically precipitated and washed 3× with 1×TBST. Bound protein from precipitated beads was eluted using acidity elution buffer (Sino Biological-provided in kit) and western blot analysis was performed.

### Co-Immunoprecipitation-Mass Spectrometry

HepG2 cells were plated to confluency on 15 cm dishes and scrapped in 10-mL PBS and collected into 15 mL conical tube. Cells in PBS were then centrifuged at 500×g for 15 min at 4° C. Supernatant was then aspirated and cell pellet was re-suspended in 300 μL co-IP lysis buffer (100 mM HEPES-pH 7.7, 250 mM KCl, 2 mM MgCl2, 2 mM EDTA, 10% glycerol, 1% digitonin) and then transferred to 1.7 mL tubes. The lysate was then sonicated for five pulses of 1 second each and incubated on ice for 30 minutes. Lysates were then centrifuged at 16,000×g for 15 min and supernatant was collected in fresh 1.7 mL tube. Protein quantification was performed using BCA (Pierce) and 2,000 μg of protein was used for co-IP. Co-IP was performed using GFP-Trap-Agarose beads (Chromotek) according to manufacturer's protocol. Eluted samples were given to the Northwestern Proteomics Core using the 100-minute gradient tandem mass spectrometry Orbitrap.

### Lactate Quantification Assay

Lactate quantification adapted from previous work (Gandhi et al., 2009). The lactate reporter system contained 200 μmol/L Amplex red (Molecular Probes), 4 units/mL lactate oxidase (Sigma), and 0.8 units/mL horseradish peroxidase (Sigma) in 50 mmol/L Tris-HCl, pH 7.4, which was deoxygenated with helium to reduce oxidation of Amplex red. The assays were incubated for 30 min, followed by measurement of fluorescence at 590 nm using excitation at 530 nm.

### Glucose-6-Phosphate Quantification Assay

Glucose-6-phosphate (G6P) quantification assay adapted from previous work (Zhu et al., 2009). Ten μl of G6P standards and extraction samples were pipetted to a 96-well plate, followed by the addition of 90 μl of a cocktail of 50 mM triethanolamine (pH 7.6), 1.0 mM MgCl2, 100 μM NADP+, 10 μM resazurin, 0.1 U/ml G6PD, and 0.2 U/ml diaphorase. The assays were incubated for 30 min, followed by measurement of fluorescence at 590 nm using excitation at 530 nm.

### In Vivo Metabolic Studies.

For all in vivo metabolic studies, age-matched WT and ΔE1HK1 littermates were used. For the glucose-tolerance test (GTT), mice were fasted for 16 hours and injected via an i.p. approach with a 20% dextrose (Millipore Sigma) solution in PBS at 2 g/kg body weight. For the insulin-tolerance test (ITT), mice were fasted for 4 hours and injected via an i.p. approach with a 0.1 U Humulin/ml PBS solution at 0.75 U/kg body weight (Lily).

### Mitochondrial and Cytosolic Subcellular Fractionation

Mitochondrial Isolation Kit for Tissue (Pierce) was used to purify mitochondrial and cytosolic protein subcellular fractions according to the Pierce manufacturer's protocol.

### HK Activity Assay

HK activity was determined, as previously described (Majewski et al., 2004). In brief, cells were plated on 12 cm dishes and allowed to attach overnight. The next day, cells were washed once with PBS and harvested by scraping and pelleted at 4000 rpm for 5 minutes. Cells were lysed by sonication, five pulses of 1 second, in 100 μl homogenization buffer: 0.2% Triton X-100, 0.5 mM EGTA, 10 mM D-(+)-glucose, 11.1 mM monothioglycerol, 45 mM Tris-HCl (pH 8.2), and 50 mM KH₂PO₄. After sonication, lysates were centrifuged at 8000 rpm for 5 minutes. HK activity was determined by the whole-cell lysate's ability to phosphorylate glucose over 2 minutes in an assay mixture with final concentrations of 50 mM triethanolamine chloride, 7.5 mM MgCl2, 0.5 mM EGTA, 11 mM monothioglycerol, 0.5 to 25 mM glucose, 6.6 mM ATP, 0.5 mg/mL NADP, and 0.5 U/mL G6PDH, pH 8.5. G6P formation was measured indirectly by NADPH production from G6PDH by measuring absorbance at 340 nm on a spectrophotometer and was normalized to protein concentration as determined by BCA protein assay kit (Fisher Scientific).

### Quantification and Statistical Analysis

### Statistical Analysis

Data are presented as mean±SEM or SD as indicated. For a two-group comparison unpaired two-tailed Student's t-tests was used. For data with multiple groups (>2) or multiple treatments a one- or two-way ANOVA was used as indicated followed by Tukey's post-hoc test to determine p-values for individual comparisons. No statistical methods were used to predetermine sample size. All statistical analysis was performed using Graphpad Prism 8.0. p<0.05 was considered to be statistically significant and is presented as *p<0.05, **p<0.01, ***p<0.001, ****p<0.0001, or ns=not significant). Survival experiment was performed in Prism 8 using comparison of survival curves with Log-rank (Mantel-Cox) test. For in vivo experiments, animals were assigned to experimental groups using simple randomization, without investigator blinding. Hierarchical clustering and heatmaps for metabolomics data were generated using MetaboAnalyst 4.0 statistical software (Chong et al., 2019).

### REFERENCES

Adeva-Andany, M. M., Pérez-Felpete, N., Fernández-Fernández, C., Donapetry-García, C., and Pazos-Garcia, C. (2016). Liver glucose metabolism in humans. Biosci. Rep. 36, e00416.

Aflalo, C., and Azoulay, H. (1998). Binding of Rat Brain Hexokinase to Recombinant Yeast Mitochondria: Effect of Environmental Factors and the Source of Porin. J. Bioenerg. Biomembr. 30, 245-255.
Alonso-Castro, A. J., and Salazar-Olivo, L. A. (2008). The anti-diabetic properties of Guazuma ulmifolia Lam are mediated by the stimulation of glucose uptake in normal and diabetic adipocytes without inducing adipogenesis. J. Ethnopharmacol. 118, 252-256.
Andrejeva, G., and Rathmell, J. C. (2017). Similarities and Distinctions of Cancer and Immune Metabolism in Inflammation and Tumors. Cell Metab. 26, 49-70.
Ardehali, H., Yano, Y., Printz, R. L., Koch, S., Whitesell, R. R., May, J. M., and Granner, D. K. (1996). Functional organization of mammalian hexokinase II. Retention of catalytic and regulatory functions in both the NH2- and COOH-terminal halves. J. Biol. Chem. 271, 1849-1852.
Azoulay-Zohar, H., Israelson, A., Abu-Hamad, S., and Shoshan-Barmatz, V. (2004). In self-defence: hexokinase promotes voltage-dependent anion channel closure and prevents mitochondria-mediated apoptotic cell death. Biochem. J. 377, 347-355.
Baardman, J., Verberk, S. G. S., Prange, K. H. M., van Weeghel, M., van der Velden, S., Ryan, D. G., Wüst, R. C. I., Neele, A. E., Speijer, D., Denis, S. W., et al. (2018). A Defective Pentose Phosphate Pathway Reduces Inflammatory Macrophage Responses during Hypercholesterolemia. Cell Rep. 25, 2044-2052.e5.
Bantug, G. R., Fischer, M., Grählert, J., Balmer, M. L., Unterstab, G., Develioglu, L., Steiner, R., Zhang, L., Costa, A. S. H., Gubser, P. M., et al. (2018). Mitochondria-Endoplasmic Reticulum Contact Sites Function as Immunometabolic Hubs that Orchestrate the Rapid Recall Response of Memory CD8+ T Cells. Immunity 48, 542-555.e6.
Bell, G. I., Burant, C. F., Takeda, J., and Gould, G. W. (1993). Structure and function of mammalian facilitative sugar transporters. J. Biol. Chem. 268, 19161-19164.
BeltrandelRio, H., and Wilson, J. E. (1992). Interaction of mitochondrially bound rat brain hexokinase with intramitochondrial compartments of ATP generated by oxidative phosphorylation and creatine kinase. Arch. Biochem. Biophys. 299, 116-124.
Calmettes, G., John, S., Weiss, J. N., and Ribalet, B. (2013). Hexokinase Isoforms and Glucose Metabolism in Adult and Neonatal Cardiac Myocytes. Biophys. J. 104, 314a.
Cárdenas, M. L., Cornish-Bowden, A., and Ureta, T. (1998). Evolution and regulatory role of the hexokinases. Biochim. Biophys. Acta BBA-Mol. Cell Res. 1401, 242-264.
Cheng, S.-C., Quintin, J., Cramer, R. A., Shepardson, K. M., Saeed, S., Kumar, V., Giamarellos-Bourboulis, E. J., Martens, J. H. A., Rao, N. A., Aghajanirefah, A., et al. (2014). mTOR- and HIF-1α-mediated aerobic glycolysis as metabolic basis for trained immunity. Science 345, 1250684.
Chong, J., Wishart, D. S., and Xia, J. (2019). Using MetaboAnalyst 4.0 for Comprehensive and Integrative Metabolomics Data Analysis. Curr. Protoc. Bioinforma. 68, e86.
Everts, B., Amiel, E., Huang, S. C.-C., Smith, A. M., Chang, C.-H., Lam, W. Y., Redmann, V., Freitas, T. C., Blagih, J., van der Windt, G. J. W., et al. (2014a). TLR-driven early glycolytic reprogramming via the kinases TBK1-IKKε supports the anabolic demands of dendritic cell activation. Nat. Immunol. 15, 323-332.
Fiek, C., Benz, R., Roos, N., and Brdiczka, D. (1982). Evidence for identity between the hexokinase-binding protein and the mitochondrial porin in the outer membrane of rat liver mitochondria. Biochim. Biophys. Acta 688, 429-440.
Fouquerel, E., Goellner, E. M., Yu, Z., Gagné, J.-P., Barbi de Moura, M., Feinstein, T., Wheeler, D., Redpath, P., Li, J., Romero, G., et al. (2014). ARTD1/PARP1 Negatively Regulates Glycolysis by Inhibiting Hexokinase 1 Independent of NAD+ Depletion. Cell Rep. 8, 1819-1831.
Garaude, J., Acín-Pérez, R., Martinez-Cano, S., Enamorado, M., Ugolini, M., Nistal-Villán, E., Hervás-Stubbs, S., Pelegrín, P., Sander, L. E., Enríquez, J. A., et al. (2016). Mitochondrial respiratory-chain adaptations in macrophages contribute to antibacterial host defense. Nat. Immunol. 17, 1037-1045.
Gardiner, N. J., Wang, Z., Luke, C., Gott, A., Price, S. A., and Fernyhough, P. (2007). Expression of hexokinase isoforms in the dorsal root ganglion of the adult rat and effect of experimental diabetes. Brain Res. 1175, 143-154.
Golshani-Hebroni, S. G., and Bessman, S. P. (1997). Hexokinase binding to mitochondria: a basis for proliferative energy metabolism. J. Bioenerg. Biomembr. 29, 331-338.
Gould, G. W., and Holman, G. D. (1993). The glucose transporter family: structure, function and tissue-specific expression. Biochem. J. 295, 329-341.
Ham, M., Lee, J.-W., Choi, A. H., Jang, H., Choi, G., Park, J., Kozuka, C., Sears, D. D., Masuzaki, H., and Kim, J. B. (2013). Macrophage Glucose-6-Phosphate Dehydrogenase Stimulates Proinflammatory Responses with Oxidative Stress. Mol. Cell. Biol. 33, 2425-2435.
Haschemi, A., Kosma, P., Gille, L., Evans, C. R., Burant, C. F., Starkl, P., Knapp, B., Haas, R., Schmid, J. A., Jandl, C., et al. (2012). The Sedoheptulose Kinase CARKL Directs Macrophage Polarization through Control of Glucose Metabolism. Cell Metab. 15, 813-826.
Hauser, D. N., Mamais, A., Conti, M. M., Primiani, C. T., Kumaran, R., Dillman, A. A., Langston, R. G., Beilina, A., Garcia, J. H., Diaz-Ruiz, A., et al. (2017). Hexokinases link DJ-1 to the PINK1/parkin pathway. Mol. Neurodegener. 12.
Huang, J.-B., Kindzelskii, A. L., and Petty, H. R. (2002). Hexokinase translocation during neutrophil activation, chemotaxis, and phagocytosis: disruption by cytochalasin D, dexamethasone, and indomethacin. Cell. Immunol. 218, 95-106.
Hughes, M. M., and O'Neill, L. A. J. (2018). Metabolic regulation of NLRP3. Immunol. Rev. 281, 88-98.
Jha, A. K., Huang, S. C.-C., Sergushichev, A., Lampropoulou, V., Ivanova, Y., Loginicheva, E., Chmielewski, K., Stewart, K. M., Ashall, J., Everts, B., et al. (2015). Network Integration of Parallel Metabolic and Transcriptional Data Reveals Metabolic Modules that Regulate Macrophage Polarization. Immunity 42, 419-430.
Jia, J., Arif, A., Terenzi, F., Willard, B., Plow, E. F., Hazen, S. L., and Fox, P. L. (2014). Target-selective Protein S-Nitrosylation by Sequence Motif Recognition. Cell 159, 623-634.
John, S., Weiss, J. N., and Ribalet, B. (2011). Subcellular Localization of Hexokinases I and II Directs the Metabolic Fate of Glucose. PLoS ONE 6.
Les Laboratoires Servier SMART.
Lindén, M., Gellerfors, P., and Nelson, B. D. (1982). Pore protein and the hexokinase-binding protein from the outer membrane of rat liver mitochondria are identical. FEBS Lett. 141, 189-192.
Magri, A., Belfiore, R., Leggio, L., Guarino, F., and Messina, A. (2017). A Synthetic Peptide from the N-Terminal of Hexokinase I Prevents the Interaction Between VDAC1 and SOD1 G93A Mutant Recovering the Viability of an ALS Cell Model. Biophys. J. 112, 349a.
Majewski, N., Nogueira, V., Robey, R. B., and Hay, N. (2004). Akt Inhibits Apoptosis Downstream of BID Cleavage via a Glucose-Dependent Mechanism Involving Mitochondrial Hexokinases. Mol. Cell. Biol. 24, 730-740.
Martinez, F. O., Gordon, S., Locati, M., and Mantovani, A. (2006). Transcriptional profiling of the human monocyte-to-macrophage differentiation and polarization: new molecules and patterns of gene expression. J. Immunol. Baltim. Md 1950 177, 7303-7311.
Mi, H., Muruganujan, A., Ebert, D., Huang, X., and Thomas, P. D. (2019). PANTHER version 14: more genomes, a new PANTHER GO-slim and improvements in enrichment analysis tools. Nucleic Acids Res. 47, D419-D426.
Middleton, R. J. (1990). Hexokinases and glucokinases. Biochem. Soc. Trans. 18, 180-183.
Mogilenko, D. A., Haas, J. T., L'homme, L., Fleury, S., Quemener, S., Levavasseur, M., Becquart, C., Wartelle, J., Bogomolova, A., Pineau, L., et al. (2019). Metabolic and Innate Immune Cues Merge into a Specific Inflammatory Response via the UPR. Cell 177, 1201-1216.e19.
Moon, J.-S., Hisata, S., Park, M.-A., DeNicola, G. M., Ryter, S. W., Nakahira, K., and Choi, A. M. K. (2015). mTORC1 induced HK1-dependent glycolysis regulates NLRP3 inflammasome activation. Cell Rep. 12, 102-115.
Mueckler, M. (1994). Facilitative glucose transporters. Eur. J. Biochem. 219, 713-725.
Nagy, C., and Haschemi, A. (2015). Time and Demand are Two Critical Dimensions of Immunometabolism: The Process of Macrophage Activation and the Pentose Phosphate Pathway. Front. Immunol. 6.
Nishizawa, T., Kanter, J. E., Kramer, F., Barnhart, S., Shen, X., Vivekanandan-Giri, A., Wall, V. Z., Kowitz, J., Devaraj, S., O'Brien, K. D., et al. (2014). Testing the Role of Myeloid Cell Glucose Flux in Inflammation and Atherosclerosis. Cell Rep. 7, 356-365.
O'Neill, L. A. J., and Pearce, E. J. (2016). Immunometabolism governs dendritic cell and macrophage function. J. Exp. Med. 213, 15-23.
Padgett, C. M., and Whorton, A. R. (1995). S-nitrosoglutathione reversibly inhibits GAPDH by S-nitrosylation. Am. J. Physiol.-Cell Physiol. 269, C739-C749.
Pastorino, J. G., Hoek, J. B., and Shulga, N. (2005). Activation of Glycogen Synthase Kinase 3β Disrupts the Binding of Hexokinase II to Mitochondria by Phosphorylating Voltage-Dependent Anion Channel and Potentiates Chemotherapy-Induced Cytotoxicity. Cancer Res. 65, 10545-10554.
Pavlova, N. N., and Thompson, C. B. (2016). The Emerging Hallmarks of Cancer Metabolism. Cell Metab. 23, 27-47.
Pearce, E. L., and Pearce, E. J. (2013). Metabolic Pathways in Immune Cell Activation and Quiescence. Immunity 38, 633-643.
Preller, A., and Wilson, J. E. (1992). Localization of the type III isozyme of hexokinase at the nuclear periphery. Arch. Biochem. Biophys. 294, 482-492.
Printz, R. L., Ardehali, H., Koch, S., and Granner, D. K. (1995). Human Hexokinase II mRNA and Gene Structure. Diabetes 44, 290-294.
Printz, R. L., Osawa, H., Ardehali, H., Koch, S., and Granner, D. K. (1997).
Hexokinase II gene: structure, regulation and promoter organization. Biochem. Soc. Trans. 25, 107-112.
Printz, R. L., Koch, S., Potter, L. R., O'Doherty, R. M., Tiesinga, J. J., Moritz, S., and Granner, D. K. (1993). Hexokinase II mRNA and gene structure, regulation by insulin, and evolution. J. Biol. Chem. 268, 5209-5219.
Puleston, D. J., Villa, M., and Pearce, E. L. (2017). Ancillary Activity: Beyond Core Metabolism in Immune Cells. Cell Metab. 26, 131-141.
Ray, A., and Dittel, B. N. (2010). Isolation of Mouse Peritoneal Cavity Cells. J. Vis. Exp.
Roberts, D. J., Tan-Sah, V. P., Smith, J. M., and Miyamoto, S. (2013). Akt phosphorylates HK-II at Thr-473 and increases mitochondrial HK-II association to protect cardiomyocytes. J. Biol. Chem. 288, 23798-23806.
Rodriguez, A. E., Ducker, G. S., Billingham, L. K., Martinez, C. A., Mainolfi, N., Suri, V., Friedman, A., Manfredi, M. G., Weinberg, S. E., Rabinowitz, J. D., et al. (2019). Serine Metabolism Supports Macrophage IL-1β Production. Cell Metab. 29, 1003-1011.e4.
Rodríguez-Prados, J.-C., Través, P. G., Cuenca, J., Rico, D., Aragonés, J., Martin-Sanz, P., Cascante, M., and Bosch, L. (2010). Substrate Fate in Activated Macrophages: A Comparison between Innate, Classic, and Alternative Activation. J. Immunol. 185, 605-614.
Rosano, C., Sabini, E., Rizzi, M., Deriu, D., Murshudov, G., Bianchi, M., Serafini, G., Magnani, M., and Bolognesi, M. (1999). Binding of non-catalytic ATP to human hexokinase I highlights the structural components for enzyme-membrane association control. Structure 7, 1427-1437.
Rose, I. A., and Warms, J. V. B. (1967). Mitochondrial Hexokinase RELEASE, REBINDING, AND LOCATION. J. Biol. Chem. 242, 1635-1645.
Sancho, D., Enamorado, M., and Garaude, J. (2017). Innate Immune Function of Mitochondrial Metabolism. Front. Immunol. 8.
Sanman, L. E., Qian, Y., Eisele, N. A., Ng, T. M., van der Linden, W. A., Monack, D. M., Weerapana, E., and Bogyo, M. (2016). Disruption of glycolytic flux is a signal for inflammasome signaling and pyroptotic cell death. ELife 5, e13663.
Saraiva, L. M., Silva, G. S. S. da, Galina, A., da-Silva, W. S., Klein, W. L., Ferreira, S. T., and Felice, F. G. D. (2010). Amyloid-β Triggers the Release of Neuronal Hexokinase 1 from Mitochondria. PLOS ONE 5, e15230.
Schägger, H. (2006). Tricine-SDS-PAGE. Nat. Protoc. 1, 16-22.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.
Sen, S., Kaminiski, R., Deshmane, S., Langford, D., Khalili, K., Amini, S., and Datta, P. K. (2015). Role of hexokinase-1 in the survival of HIV-1-infected macrophages. Cell Cycle Georget. Tex 14, 980-989.
Shoshan-Barmatz, V., Zakar, M., Rosenthal, K., and Abu-Hamad, S. (2009). Key regions of VDAC1 functioning in apoptosis induction and regulation by hexokinase. Biochim. Biophys. Acta BBA-Bioenerg. 1787, 421-430.
Shoshan-Barmatz, V., De Pinto, V., Zweckstetter, M., Raviv, Z., Keinan, N., and Arbel, N. (2010). VDAC, a multi-functional mitochondrial protein regulating cell life and death. Mol. Aspects Med. 31, 227-285.
Stienstra, R., Netea-Maier, R. T., Riksen, N. P., Joosten, L. A. B., and Netea, M. G. (2017). Specific and Complex Reprogramming of Cellular Metabolism in Myeloid Cells during Innate Immune Responses. Cell Metab. 26, 142-156.
Sui, D., and Wilson, J. E. (1997). Structural Determinants for the Intracellular Localization of the Isozymes of Mammalian Hexokinase: Intracellular Localization of Fusion Constructs Incorporating Structural Elements from the Hexokinase Isozymes and the Green Fluorescent Protein. Arch. Biochem. Biophys. 345, 111-125.
Sun, L., Shukair, S., Naik, T. J., Moazed, F., and Ardehali, H. (2008). Glucose Phosphorylation and Mitochondrial Binding Are Required for the Protective Effects of Hexokinases I and II. Mol. Cell. Biol. 28, 1007-1017.
Thomsen, L. L., Scott, J. M. J., Topley, P., Knowles, R. G., Keerie, A.-J., and Frend, A. J. (1997). Selective Inhibition of Inducible Nitric Oxide Synthase Inhibits Tumor Growth in Vivo: Studies with 1400W, a Novel Inhibitor. Cancer Res. 57, 3300-3304.
Tyson, R. L., Perron, J., and Sutherland, G. R. (2000). 6-Aminonicotinamide inhibition of the pentose phosphate pathway in rat neocortex. Neuroreport 11, 1845-1848.
Ureta, T. (1982). The comparative isozymology of vertebrate hexokinases. Comp. Biochem. Physiol. B 71, 549-555.
Wang, J., Zhang, X., Ma, D., Lee, W.-N. P., Xiao, J., Zhao, Y., Go, V. L., Wang, Q., Yen, Y., Recker, R., et al. (2013). Inhibition of transketolase by oxythiamine altered dynamics of protein signals in pancreatic cancer cells. Exp. Hematol. Oncol. 2.
Wang, S., Song, R., Wang, Z., Jing, Z., Wang, S., and Ma, J. (2018). S100A8/A9 in Inflammation. Front. Immunol. 9.
Wilson, J. E. (1995). Hexokinases. Rev. Physiol. Biochem. Pharmacol. 126, 65-198.
Wilson, J. E. (2003). Isozymes of mammalian hexokinase: structure, subcellular localization and metabolic function. J. Exp. Biol. 206, 2049-2057.
Wolf, A. J., Reyes, C. N., Liang, W., Becker, C., Shimada, K., Wheeler, M. L., Cho, H. C., Popescu, N. I., Coggeshall, K. M., Arditi, M., et al. (2016). Hexokinase Is an Innate Immune Receptor for the Detection of Bacterial Peptidoglycan. Cell 166, 624-636.
Zeng, C., Aleshin, A. E., Hardie, J. B., Harrison, R. W., and Fromm, H. J. (1996). ATP-Binding Site of Human Brain Hexokinase As Studied by Molecular Modeling and Site-Directed Mutagenesis^{†}. Biochemistry 35, 13157-13164.
Zhang, D., Yip, Y. M., and Li, L. (2016). In silico construction of HK2-VDAC1 complex and investigating the HK2 binding-induced molecular gating mechanism of VDAC1. Mitochondrion 30, 222-228.
Zhang, W., Wang, G., Xu, Z.-G., Tu, H., Hu, F., Dai, J., Chang, Y., Chen, Y., Lu, Y., Zeng, H., et al. (2019). Lactate Is a Natural Suppressor of RLR Signaling by Targeting MAVS. Cell 178, 176-189.e15.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating an immunodeficiency disease or disorder in a subject in need thereof, the method comprising administering to the subject an effective amount of a therapeutic agent that results in dissociation of hexokinase 1 (HK1) from the outer membrane of mitochondria and into the cytosol of macrophage cells in the subject.

2. The method of claim 1, wherein the immunodeficiency disease or disorder is selected from HIV infection, ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglubulinemia, Bruton's disease, congenital agammaglobulinemia, selective deficiency of IgA, and Wiskott-Aldrich syndrome.

3. The method of claim 1, wherein the immunodeficiency disease or disorder is the result of a family history of primary immunodeficiency in the subject in need thereof.

4. The method of claim 1, wherein the immunodeficiency disease or disorder is the result of spleen removal in the subject.

5. The method of claim 1, wherein the immunodeficiency disease or disorder is the result of cancer or liver cirrhosis in the subject.

6. The method of claim 1, wherein the therapeutic agent is selected from clotrimazole, bifonazole, econazole, ketoconazole, miconazole, and tioconazole.

7. The method of claim 6, wherein the therapeutic agent is clotrimazole.

8. The method of claim 7, wherein the effective amount of clotrimazole induces elevated production of IL-1β, TNFα, and IL-6 by the macrophage cells.

9. The method of claim 7, wherein the effective amount of clotrimazole inhibits glyceraldehyde-3-phosphate dehydrogenase (GAPDH) activity in the macrophage cells.

10. The method of claim 7, the effective amount of clotrimazole results in an increase of activity of the pentose phosphate pathway (PPP) in the macrophage cells.

11. The method of claim 7, wherein the effective amount of clotrimazole induces a hyper-inflammatory response in the subject.

12. A method for treating an immunodeficiency disease or disorder in a subject in need thereof, the method comprising administering to the subject an effective amount of a therapeutic agent that inhibits the activity of glyceraldehyde-3-phosphate dehydrogenase (GAPDH).

13. The method of claim 12, wherein the therapeutic agent is selected from CGP 3466 maleate (CAS 200189-97-5), heptelidic acid, deprenyl, and dihydromanumycin A.

14. A method for elevating production of one or more inflammatory cytokines in a subject in need thereof, the method comprising administering to the subject an effective amount of a therapeutic agent that results in dissociation of hexokinase 1 (HK1) from the outer membrane of mitochondria and into the cytosol of macrophage cells in the subject.

15. The method of claim 14, wherein the one or more inflammatory cytokines are selected from IL-1β, TNFα, IL-6, and combinations thereof.

16. The method of claim 14, wherein the agent is selected from clotrimazole, bifonazole, econazole, ketoconazole, miconazole, and tioconazole.

17. The method of claim 16, wherein the therapeutic agent is clotrimazole.

18. A method for elevating production of one or more inflammatory cytokines in a subject in need thereof, the method comprising administering to the subject an effective amount of a therapeutic agent that inhibits the activity of glyceraldehyde-3-phosphate dehydrogenase (GAPDH).

19. The method of claim 18, wherein the one or more inflammatory cytokines are selected from IL-1β, TNFα, IL-6, and combinations thereof.

20. The method of claim 18, wherein the therapeutic agent is selected from CGP 3466 maleate (CAS 200189-97-5), heptelidic acid, deprenyl, and dihydromanumycin A.


SEQUENCE LISTING (9 sequences)

SEQ ID NO: 1 - protein, 9 aa, Artificial Sequence
  Synthetic methionine-FLAG-tag
MDYKDDDDK

SEQ ID NO: 2 - protein, 11 aa, Mus musculus
ALNSIIDVYHK

SEQ ID NO: 3 - protein, 7 aa, Mus musculus
GADVWFK

SEQ ID NO: 4 - protein, 11 aa, Mus musculus
LLETECPQYIR

SEQ ID NO: 5 - protein, 13 aa, Mus musculus
LGHPDTLNQGEFK

SEQ ID NO: 6 - protein, 15 aa, Mus musculus
NIETIINTFHQYSVK

SEQ ID NO: 7 - protein, 15 aa, Mus musculus
VIEHIMEDLDTNADK

SEQ ID NO: 8 - protein, 8 aa, Mus musculus
LTWASHEK

SEQ ID NO: 9 - protein, 7 aa, Mus musculus
DLQNFLK